(19) United States
(12) Patent Application Publication
(10) Pub. No.: US 2026/0258003 A1
(43) Pub. Date: Sep. 3, 2026

(54) SLACK-ACTIVATING COMPOUNDS AND THEIR MEDICAL USE

(71) Applicant: Johann Wolfgang Goethe-Universität Frankfurt am Main, Frankfurt am Main (DE)

(72) Inventors: ACHIM SCHMIDTKO, Frankfurt am Main (DE); EUGEN PROSCHAK, Friedrichsdorf (DE); ANNIKA BALZULAT, Frankfurt am Main (DE); WENXIN ZHU, Langen (DE); CATHRIN FLAUAUS, Frankfurt am Main (DE); DIETER STEINHILBER, Schmitten (DE); RUIRUI LU, Frankfurt am Main (DE); VICTOR HERNANDEZ OLMOS, Frankfurt am Main (DE); JAN HEERING, Frankfurt am Main (DE)

(21) Appl. No.: 19/163,300

(22) PCT Filed: Mar. 8, 2024
(86) PCT No.: PCT/EP2024/056287

§ 371 Date: Sep. 8, 2025

(30) Foreign Application Priority Data
Mar. 8, 2023 (EP) 23160797.9
May 3, 2023 (EP) 23171431.2

(51) Int. Cl. C07D 267/20; A61K 31/167; A61K 31/24; A61K 31/4439; A61K 31/553; A61K 31/554; A61P 17/04; A61P 29/00; C07C 205/38; C07C 217/90; C07C 233/75; C07D 281/16; C07D 413/12; C07D 413/14

(52) U.S. Cl. CPC C07D 267/20; A61K 31/167; A61K 31/24; A61K 31/4439; A61K 31/553; A61K 31/554; A61P 17/04; A61P 29/00; C07C 205/38; C07C 217/90; C07C 233/75; C07D 281/16; C07D 413/12; C07D 413/14

(57) ABSTRACT

The present invention pertains to novel compounds and their synthesis which are structurally derived from 2-chloro-11-(4-methylpiperazin-1-yl)dibenzo[b,f][1,4]oxazepine. The compounds of the invention were shown in a comparative study to bind and activate potassium channels, in particular Slack. Based on their activating activity, the compounds of the invention and pharmaceutical compositions containing them can be used in various therapeutic applications for the treatment or prevention of pathologies.

### FIELD OF THE INVENTION

The present invention pertains to novel compounds and their synthesis which are structurally derived from 2-chloro-11-(4-methylpiperazin-1-yl)dibenzo[b,f][1,4]oxazepine. The compounds of the invention were shown in a comparative study to bind and activate potassium channels, in particular Slack. Based on their activating activity, the compounds of the invention and pharmaceutical compositions containing them can be used in various therapeutic applications for the treatment or prevention of pathologies. The compounds of the invention are of particular use in the treatment of systemic pain/inflammatory pain and/or pruritus/topical itch.

### DESCRIPTION

Itch (also known as pruritus) is a debilitating symptom that accompanies various skin disorders, systemic diseases such as chronic kidney or cholestatic liver disease, psychiatric diseases, and neuropathies, or is idiopathic with unknown reason. It is defined as an unpleasant sensation that evokes a desire to scratch and affects about one third of dermatology patients and nearly 15% of the general population^{1,2}, Like pain, acute itch serves as an important protective mechanism to detect potentially harmful stimuli. However, chronic itch (i.e., persisting for more than 6 weeks in humans) does not serve a useful function but instead imposes suffering and may compromise quality of life to a degree often comparable to that for chronic pain.

Itch can be categorized into histamine-dependent (histaminergic) and histamine-independent (non-histaminergic) itch. As most types of chronic itch are histamine-independent and therefore resistant to antihistamines, there is an urgent need to develop novel treatment strategies.

Most known mechanisms of pruriception (itch sensation) begin with the activation of itch-sensitive sensory neurons. Several key receptors, including members of the Mas-related G-protein-coupled receptor (Mrgpr) families, were found to be important for detecting non-histaminergic chemical itch signals³. Recent single-cell RNA-sequencing (scRNA-seq) studies have revealed that itch-related genes are highly expressed in distinct subpopulations of sensory neurons. For example, in a pioneering scRNA-seq study by Usoskin and colleagues 11 principal types of sensory neurons have been identified, of which 3 non-peptidergic populations (termed NP1, NP2 and NP3) were proposed to be itch-sensitive⁴. After activation, these sensory neurons signal to the dorsal horn of the spinal cord, where the ongoing information is further processed and transmitted to itch-signaling pathways ascending to the brain⁵.

The excitability of sensory neurons is driven by various types of ion channels, among which K⁺ channels are the most populous and diverse class that are governed by more than 75 genes in humans and feature tissue-dependent expression patterns⁶ Notably, the K⁺ channel Slack (also termed K_{Na}1.1; gene Kcnt1), which is activated by intracellular Na⁺ and inhibited by bivalent cations^{7,8}, is highly enriched in the itch-sensitive NP1, NP2 and NP3 subsets of sensory neurons in mice^{4,9} (FIG. 1A), suggesting that Slack has a role in itch. In fact, a significant functional role of Slack in pruriception is reflected by the increased scratching behavior of Slack knockout mice after exposure to pruritogens such as chloroquine and histamine¹⁰. These findings in combination with the observation that Slack expression in non-human primate sensory neurons¹¹ (FIG. 1C) and in human sensory neurons¹² (FIG. 1B) is enriched in itch-associated cell populations support the hypothesis that activators of Slack channels hold therapeutic potential for treatment of itch.

A previous study with a library screen of pharmacologically active compounds reported that the first-generation antipsychotic drug Loxapine activates Slack¹³. In preliminary experiments, the inventors observed that systemic administration of a low dose of Loxapine significantly alleviated chloroquine-induced scratching behavior of wildtype mice but did not affect the scratching behavior in Slack knockout mice (data not shown), indicating that the Loxapine-induced antipruritic effect depends on Slack activation.

Despite the need for drugs to treat histamine-independent itch, the clinical use of Loxapine is unfortunately limited by the typical adverse events of first-generation antipsychotic drugs that are related to blocking activities at dopamine receptors and other receptors^{14,15}.

Starting from the structure of Loxapine, inventors developed novel structural Loxapine derivatives that have an activating effect on Slack. Notably, the Loxapine derivatives of the present invention feature a different and more favorable pharmacological profile than Loxapine. Further, the compounds of the invention, which act as Slack activators, have comparable or improved binding properties to Slack, a reduced BBB permeability, which translates to less unwanted side effects, and improved off target activity in comparison to the parent molecule Loxapine.

### BRIEF DESCRIPTION OF THE INVENTION

Generally, and by way of brief description, the main aspects of the present invention can be described as follows:

In a first aspect, the invention pertains to a compound, wherein the compound has the formula I:

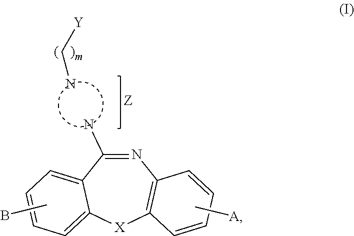

or salt, complex, diastereomer, enantiomer and/or tautomer of a compound with the formula (I), wherein
A is one or two substituents independently selected from —H, —F, —Cl, —Br, —I, —CF₃, —OCF₃, —CF₂H, and —OCF₂H;
B is one or two substituents independently selected from —F, —Cl, —Br, —I, —CF₃, —OCF₃, —CF₂H, and —OCF₂H;
X is selected from —S— and —O—;
m is 0 to 3;
Y is selected from
   —H;
   a nitrile group,
   a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O; or
   a linear —C₁₋₃alkyl, wherein a, preferably one, —(CH₂)— of the linear —C₁₋₃alkyl is optionally monosubstituted with —OH, and wherein the terminal —CH₃ of the linear —C₁₋₃alkyl is monosubstituted with —OR₁ or —SR₁, preferably —OR₁, wherein R¹ is —H, —(CH₂)—COOH or —(CH₂)—(CH₂)—OH; or
   —(CH₂)ᵢ—CX¹R², wherein
      X¹ is ═NR³ or ═O, wherein R³ is —H or —OH.
      i is 0 or 1, and
      R² is —OR⁴ or —NR⁵R⁶, wherein R⁴ is —H or —C₁₋₂alkyl; wherein R⁵ and R⁶ are independently selected from —H or -methyl;

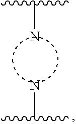

herein denoted as “Z”, is an aliphatic group comprising at least one, preferably 1 or 2, heterocycle(s) which comprise together two nitrogen atoms,
wherein the first of the two nitrogen atoms connects Z to

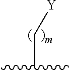

of formula I and the second of the two nitrogen atom connects Z to

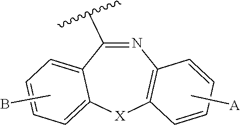

of formula I, preferably to

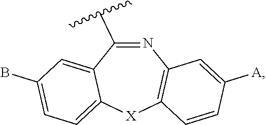

and wherein within group Z:
   the two nitrogen atoms are each a heteroatom in one of two connected 4-membered heterocycles (each heterocycle having no more than one heteroatom), wherein the two connected 4-membered heterocycles form a spirocycle, preferably wherein the 4-membered heterocycle with the first nitrogen atom is connected via a shared carbon atom to the 4-membered heterocycle with the second nitrogen atom, thus forming a spirocycle with two heteroatoms that are the two nitrogen atoms; or
   the first of the two nitrogen atoms is connected to a 4-membered heterocycle with one heteroatom, wherein the second of the two nitrogen atoms is the heteroatom in wherein the first nitrogen atom is further substituted with methyl; or
   the two nitrogen atoms are heteroatoms in a 6- to 7-membered heterocycle with two heteroatoms, wherein the heterocycle is optionally substituted, preferably monosubstituted, with methyl.

In a second aspect, the invention pertains to a pharmaceutical composition comprising the compound of any one of the aspects herein, or a salt, solvate, or ester thereof, and a pharmaceutically acceptable carrier or excipient.

In a third aspect, the invention pertains to a compound or composition for use in the treatment of a condition in a subject, the compound or composition comprising an effective amount of the compound of the first aspect of the invention, or a salt, solvate, or ester thereof, wherein the condition is treatable by activating a potassium channel in a cell associated with the pathology of the condition, the treatment comprising administering the compound or composition to the subject in need thereof.

In a fourth aspect, the invention pertains to a method of synthesizing a compound of the first aspect of the invention.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a compound, wherein the compound has the formula I:

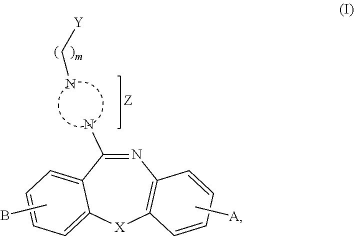

or salt, complex, diastereomer, enantiomer and/or tautomer of a compound with the formula (I), wherein
A is one or two substituents independently selected from —H, —F, —Cl, —Br, —I, —CF₃, —OCF₃, —CF₂H, and —OCF₂H;
B is one or two substituents independently selected from —F, —Cl, —Br, —I, —CF₃, —OCF₃, —CF₂H, and —OCF₂H;
X is selected from —S— and —O—;
m is 0 to 3;
Y is selected from
   —H;
   a nitrile group;
   a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O; or
   a linear —C₁₋₃alkyl, wherein a, preferably one, —(CH₂)— of the linear —C₁₋₃alkyl is optionally monosubstituted with —OH, and wherein the terminal —CH₃ of the linear —C₁₋₃alkyl is monosubstituted with —OR¹ or —SR¹, preferably —OR¹, wherein R¹ is H, —(CH₂)—COOH or —(CH₂)—(CH₂)—OH; or
   —(CH₂)ᵢ—CX¹R², wherein
      X¹ is ═NR³ or ═O, wherein R³ is —H or —OH.
      i is 0 or 1, and
      R² is —OR⁴ or —NR⁵R⁶, wherein R⁴ is —H or —C₁₋₂alkyl; wherein R⁵ and R⁶ are independently selected from —H or -methyl.

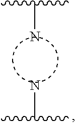

herein denoted as “Z”, is an aliphatic group comprising at least one, preferably 1 or 2, heterocycle(s) which comprise together two nitrogen atoms,
wherein the first of the two nitrogen atoms connects Z to

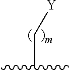

of formula I and the second of the two nitrogen atom connects Z to

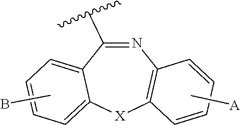

of formula I, preferably to

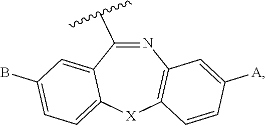

and wherein within group Z:
   the two nitrogen atoms are each a heteroatom in one of two connected 4-membered heterocycles (each heterocycle having no more than one heteroatom), wherein the two connected 4-membered heterocycles form a spirocycle, preferably wherein the 4-membered heterocycle with the first nitrogen atom is connected via a shared carbon atom to the 4-membered heterocycle with the second nitrogen atom, thus forming a spirocycle with two heteroatoms that are the two nitrogen atoms; or
   the first of the two nitrogen atoms is connected to a 4-membered heterocycle with one heteroatom, wherein the second of the two nitrogen atoms is the heteroatom in wherein the first nitrogen atom is further substituted with methyl; or
   the two nitrogen atoms are heteroatoms in a 6- to 7-membered heterocycle with two heteroatoms, wherein the heterocycle is optionally substituted, preferably monosubstituted, with methyl.

In preferred embodiments of the invention, the compound is not

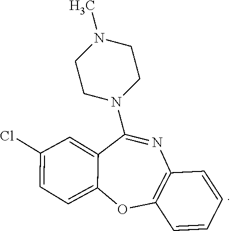 [SMILES: CN1CCN(C2=Nc3ccccc3Oc3ccc(Cl)cc32)CC1]

In preferred embodiments of the invention, the compound is a salt, complex, diastereomer, enantiomer and/or tautomer of a compound with the formula (I).

In the context of the invention, when a first chemical group is “substituted” with a second chemical group, this indicates that the second chemical group is bound to the first chemical group, preferably by replacing a hydrogen atom of the first chemical group at the position where the respective bond formed between the first and the second chemical group. In this context, the term “substituted” does not indicate the nature of the chemical reaction. Similarly, if a first chemical group is a “substituent” of a second chemical group, this indicates that the chemical groups are attached to each other.

The term “carbocyclic”, “carbocycle” or “carbocyclyl” includes, unless otherwise indicated, in general a 3- to 9-membered, preferably a 4- to 8-membered, a 3- to 6-membered or a 5- to 7-membered, more preferably a 5- or 6-membered monocyclic ring comprising 3 to 9, preferably 4 to 8, 3 to 6 or 5 to 7, more preferably 5 or 6 ring carbon atoms. In preferred embodiments, a carbocycle according to the invention comprises 4, 5, 6, or 7 ring carbon atoms. The carbocycle may be saturated, partially or fully unsaturated, or aromatic, wherein saturated means that only single bonds are present, and partially or fully unsaturated means that one or more double bonds may be present in suitable positions, while the Huckel rule for aromaticity is not fulfilled, whereas aromatic means that the Huckel (4n+2) rule is fulfilled. The term “carbocycle”, “carbocyclic” or “carbocyclyl” may therefore cover inter alia cycloalkyl, cycloalkenyl, as well as phenyl. Preferably, the term “carbocyclic”, “carbocyclyl” or “carbocycle” covers cycloalkyl and cycloalkenyl groups, for example cyclopropane, cyclobutane, cyclopentane and cyclohexane rings. In preferred embodiments, the “carbocycle”, “carbocyclic” or “carbocyclyl” is a non-aromatic “carbocycle”, “carbocyclic” or “carbocyclyl”.

The term “cycloalkyl” as used herein, unless otherwise indicated, denotes in each case a monocyclic cycloaliphatic group having usually from 3 to 9 or from 4 to 7 ring carbon atoms and no ring heteroatoms. Examples of such a cycloalkyl include cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “aryl” or “aromatic carbocycle” preferably includes 6-membered aromatic carbocyclic rings based on carbon atoms as ring members. A preferred example is phenyl.

The term “heterocyclic”, “heterocycle” or “heterocyclyl” includes, unless otherwise indicated, in general a 3- to 9-membered, preferably a 4- to 7-membered, preferably in each case a 5- to 7-membered, 5- to 6-membered, 6- to 7-membered, or a 3- to 5-membered, in particular a 4-5-, 6- or 7-membered monocyclic ring. The heterocycle typically comprises one or more, e.g. 1, 2, 3, or 4, preferably 1, 2, or 3, preferably 1 or 2 heteroatoms, selected from N, O, and S as ring members, where S-atoms as ring members may be present as S, SO or SO₂, where N-atoms as ring members may be present as N or NH, where O as ring members may be present as O. Preferably, the heteroatom is a N-atom. The remaining ring members are carbon atoms.

In the context of the invention, when referring to hetero atoms in a heterocycle, the heteroatom “N” preferably refers to a ring member present in the form of N, or NH, the heteroatom “O” preferably refers to a ring member present in the form of O, the heteroatom S preferably refers to a ring member present in the form of S, SO or SO₂.

The heterocyclyl may be saturated, partially or fully unsaturated, or aromatic, wherein saturated means that only single bonds are present, and partially or fully unsaturated means that one or more double bonds may be present in suitable positions, while the Huckel rule for aromaticity is not fulfilled, whereas aromatic means that the Huckel (4n+2) rule is fulfilled.

The term “heterocycloalkyl” as used herein, unless otherwise indicated, denotes a heterocyclyl as defined above, wherein the monocyclic ring is a monocyclic cycloaliphatic group having usually from 3 to 9 or from 4 to 7 ring atoms, in each case 5 to 7 ring atoms, 5 to 6 ring atoms, 6 to 7 ring atoms, 3 to 5 ring atoms, in particular a 4-5-, 6- or 7 ring atoms, comprising as ring members one or more, e.g. 1, 2, 3 or 4, preferably 1, 2, or 3, preferably 1 or 2 heteroatoms, selected from N, O and S as ring members, where S-atoms as ring members may be present as S, SO or SO₂, where N-atoms as ring members may be present as N or NH, where O as ring members may be present as O. The remaining ring members are carbon atoms. Preferably, the term “heterocycloalkyl” includes, unless otherwise indicated, a 3- to 9-membered, preferably a 4-to 7-membered, preferably in each case a 5- to 7-membered, 5- to 6-membered, 6- to 7-membered, or a 3- to 5-membered, in particular a 4-5-, 6- or 7-membered monocyclic ring.

The term “hetaryl” or “heteroaryl” or “aromatic heterocycle” or “aromatic heterocyclic ring”, unless otherwise indicated, includes monocyclic 5- or 6-membered aromatic heterocycles comprising as ring members 1, 2, 3 or 4 heteroatoms. In preferred embodiments, the heterocyclyl is a non-aromatic heterocyclyl.

The term “spirocycle” as used herein, if not indicated otherwise, refers to a group with at least two, preferably two, monocyclic rings, wherein two of the at least two monocyclic rings are connected by a shared atom which is a member of both connected monocyclic rings.

The term “carbospirocycle” as used herein, if not indicated otherwise, refers to a group with at least two, preferably two, carbocycles, wherein two of the at least two carbocycles are connected by a shared atom which is a member of both connected carbocycles.

The term “heterospirocycle” as used herein, if not indicated otherwise, refers to a group with at least two, preferably two, monocyclic rings, wherein the monocyclic rings are connected by a shared atom which is a member of both connected monocyclic rings, and wherein at least one of the monocyclic rings is a heterocycle.

A spirocycle in the context of the invention preferably contains at least one, preferably 1 or 2, heteroatoms, wherein the heteroatoms are preferably N. Preferably, a spirocycle in the context of the invention comprises two monocyclic rings with 4 ring atoms each.

In the context of the invention, the term “alkyl” refers to a saturated straight or branched hydrocarbon. For example, alkyl may in certain embodiments refer to a methyl, ethyl, propyl, isopropyl (also called 2-propyl or 1-methylethyl), butyl, isobutyl, tert-butyl, n-pentyl, iso-pentyl, sec-pentyl, neo-pentyl, 1,2-dimethyl-propyl, iso-amyl, n-hexyl, isohexyl, sec-hexyl, n-heptyl, iso-heptyl, n-octyl, 2-ethyl-hexyl, n-nonyl, n-decyl, n-undecyl, n-dodecyl.

In preferred embodiments, m is 0 to 2, more preferably 0 or 1, most preferably m is 1. In preferred embodiments, m is 0.

In preferred embodiments, Y is not-H. In preferred embodiments, Y is selected from
a nitrile group,
a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O;
a linear —C₁₋₃alkyl, wherein a, preferably one, —(CH₂)— of the linear —C₁₋₃alkyl is optionally monosubstituted with —OH, and wherein the terminal —CH₃ of the linear —C₁₋₃alkyl is monosubstituted with —OR¹ or —SR¹, preferably —OR¹, wherein R¹ is H, —(CH₂)—COOH or —(CH₂)—(CH₂)—OH; or
(CH₂)ᵢ—CX¹R², wherein
   X¹ is ═NR³ or ═O, wherein R³ is —H or —OH.
   i is 0 or 1, and
   R² is —OR⁴ or —NR⁵R⁶, wherein R⁴ is —H or —C₁₋₂alkyl; wherein R⁵ and R⁶ are independently selected from —H or -methyl.

In preferred embodiments, Y is not-H. In preferred embodiments, Y is not a nitrile group. In preferred embodiments, Y is selected from
a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O;
a linear —C₁₋₃alkyl, wherein a, preferably one, —(CH₂)— of the linear —C₁₋₃alkyl is optionally monosubstituted with —OH, and wherein the terminal —CH₃ of the linear —C₁₋₃alkyl is monosubstituted with —OR¹ or —SR¹, preferably —OR¹, wherein R¹ is H, —(CH₂)—COOH or —(CH₂)—(CH₂)—OH; or
—(CH₂)ᵢ—CX¹R², wherein
   X¹ is ═NR³ or ═O, wherein R³ is —H or —OH.
   i is 0 or 1, and
   R² is —OR⁴ or —NR⁵R⁶, wherein R⁴ is —H or —C₁₋₂alkyl; wherein R⁵ and R⁶ are independently selected from —H or -methyl.

In preferred embodiments, Y is not-H. In preferred embodiments, Y is selected from
a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O; or
a linear —C₁₋₃alkyl, wherein a, preferably one, —(CH₂)— of the linear —C₁₋₃alkyl is optionally monosubstituted with —OH, and wherein the terminal —CH₃ of the linear —C₁₋₃alkyl is monosubstituted with —OR¹ or —SR¹, preferably —OR¹, wherein R¹ is H, —(CH₂)—COOH or —(CH₂)—(CH₂)—OH.

In preferred embodiments, Y is —H.

In preferred embodiments, Y is a nitrile group.

In preferred embodiments, Y is a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O.

In preferred embodiments, Y is selected from a linear —C₁₋₃alkyl, wherein a —(CH₂)— of the linear —C₁₋₃alkyl is optionally monosubstituted with —OH, and wherein the terminal —CH₃ of the linear —C₁₋₃alkyl is monosubstituted with —OR¹ or —SR¹, preferably —OR¹, wherein R¹ is —H, —(CH₂)—COOH or —(CH₂)—(CH₂)—OH. More preferably, Y is selected from a linear —C₁₋₂alkyl, wherein a —(CH₂)— of the linear —C₁₋₂alkyl is optionally monosubstituted with —OH, and wherein the terminal —CH₃ of the linear —C₁₋₂alkyl is monosubstituted with —OR¹ or —SR¹, preferably —OR¹, wherein R¹ is H, —(CH₂)—COOH or —(CH₂)—(CH₂)—OH. Most preferably, Y is a methyl group that is monosubstituted with —OR¹ or —SR¹, preferably —OR¹, wherein R¹ is H, —(CH₂)—COOH or —(CH₂)—(CH₂)—OH.

In preferred embodiments, Y is selected from a linear —C₁₋₃alkyl, wherein a —(CH₂)— of the linear —C₁₋₃alkyl is optionally monosubstituted with —OH, and wherein the terminal —CH₃ of the linear —C₁₋₃alkyl is monosubstituted with —OR¹ or —SR¹, preferably —OR¹, wherein R¹ is —H, or —(CH₂)—COOH. More preferably, Y is selected from a linear —C₁₋₂alkyl, wherein a —(CH₂)— of the linear —C₁₋₂alkyl is optionally monosubstituted with —OH, and wherein the terminal —CH₃ of the linear —C₁₋₂alkyl is monosubstituted with —OR¹ or —SR¹, preferably —OR¹, wherein R¹ is H, or —(CH₂)—COOH. Most preferably, Y is a methyl group that is monosubstituted with —OR¹ or —SR¹, preferably-OR¹, wherein R¹ is H, or —(CH₂)—COOH.

In preferred embodiments, Y is selected from a linear —C₁₋₃alkyl, wherein a —(CH₂)— of the linear —C₁₋₃alkyl is optionally monosubstituted with —OH, and wherein the terminal —CH₃ of the linear —C₁₋₃alkyl is monosubstituted with —OR¹ or —SR¹, preferably —OR¹, wherein R¹ is or —(CH₂)—COOH. More preferably, Y is selected from a linear —C₁₋₂alkyl, wherein a —(CH₂)— of the linear —C₁₋₂alkyl is optionally monosubstituted with —OH, and wherein the terminal —CH₃ of the linear —C₁₋₂alkyl is monosubstituted with —OR¹ or —SR¹, preferably —OR¹, wherein R¹ is —(CH₂)—COOH. Most preferably, Y is a methyl group that is monosubstituted with —OR¹ or —SR¹, preferably —OR¹, wherein R¹ is —(CH₂)—COOH.

In preferred embodiments, Y is —(CH₂)ᵢ—CX¹R², wherein
X¹ is ═NR³ or ═O, wherein R³ is —H or —OH; i is 0 or 1, and
R² is —OR⁴ or —NR⁵R⁶; wherein R⁴ is —H or —C₁₋₂alkyl; wherein R⁵ and R⁶ are independently selected from —H or -methyl.

In preferred embodiments, i is 0.

In preferred embodiments, R³ is —OH.

In preferred embodiments, R⁵ and R⁶ are both-H.

In preferred embodiments, i is 0, X¹ is ═NR³, R³ is —OH, R² is —NR⁵R⁶ and R⁵ and R⁶ are both-H.

In preferred embodiments, i is 0 or 1, X¹ is ═O, R² is —NR⁵R⁶ and R⁵ and R⁶ are both-H.

In preferred embodiments, X¹ is ═O and R² is —OR⁴, wherein R⁴ is —H or —C₁₋₂alkyl; preferably H.

In preferred embodiments, when referring to Y, the 5- to 6-membered unsaturated heterocycle comprising 3 hetero atoms that are nitrogen is
a 5-membered unsaturated heterocycle comprising 3 hetero atoms that are selected from O and N, preferably wherein 2 or 3 heteroatoms are nitrogen, wherein the 5-membered unsaturated heterocycle is monosubstituted with ═S or ═O, preferably with ═O;
a 6-membered heterocycle comprising 3 hetero atoms that are nitrogen, wherein the 6-membered heterocycle is di-substituted with ═O or ═S, preferably with ═O.

In preferred embodiments, when referring to Y, the 5- to 6-membered unsaturated heterocycle comprising 3 hetero atoms that are nitrogen is a 5-membered heterocycle comprising 3 hetero atoms that are nitrogen, wherein the 5-membered heterocycle is monosubstituted with ═O.

In preferred embodiments, Y is selected from the group consisting of

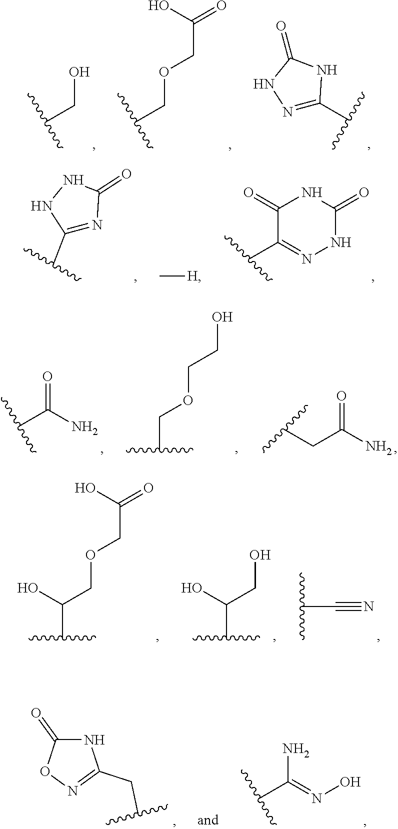

more preferably, wherein Y selected from the group consisting of

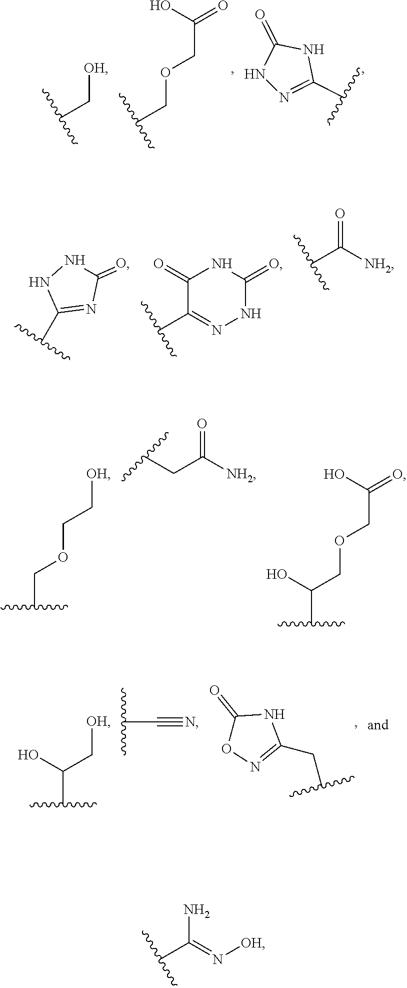

even more preferably, wherein Y is selected from the group consisting of

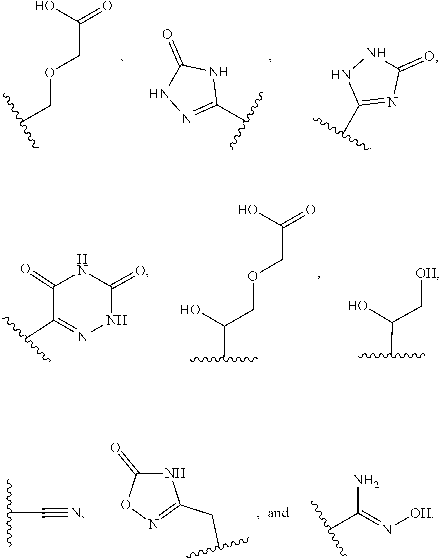

In more preferred embodiments, Y is selected from the group consisting of:

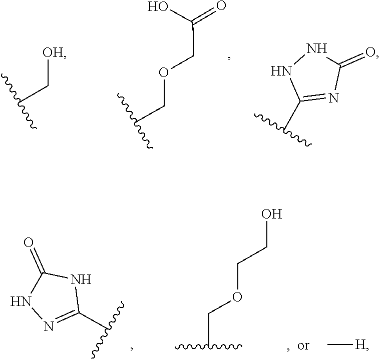

more preferably, wherein Y is selected from the group consisting of

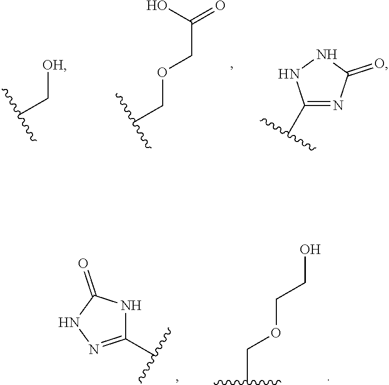

In more preferred embodiments, Y is selected form the group consisting of

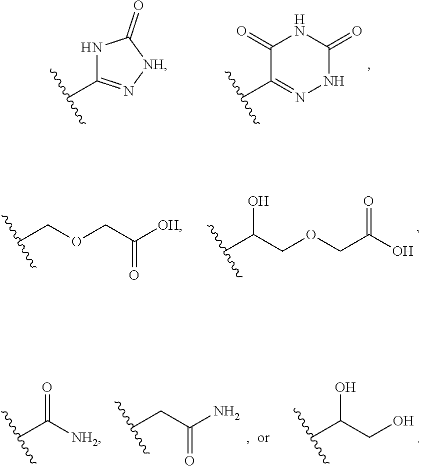

In preferred embodiments, Z is an aliphatic group comprising two nitrogen atoms and 1 heterocycle, wherein the first of the two nitrogen atoms connects Z to

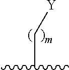

and the second of the two nitrogen atom connects Z to

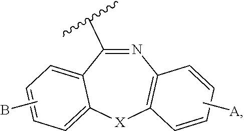

preferably to

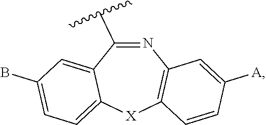

and the two nitrogen atoms are heteroatoms in a 6- to 7-membered heterocycle with two heteroatoms, wherein the heterocycle is optionally substituted, preferably monosubstituted, with methyl. In preferred embodiments, said 6- to 7-membered heterocycle with two heteroatoms is a 6-membered heterocycle with two heteroatoms. In other preferred embodiments, said 6- to 7-membered heterocycle with two heteroatoms is a 7-membered heterocycle with two heteroatoms.

In preferred embodiments, Z is selected a group consisting of:

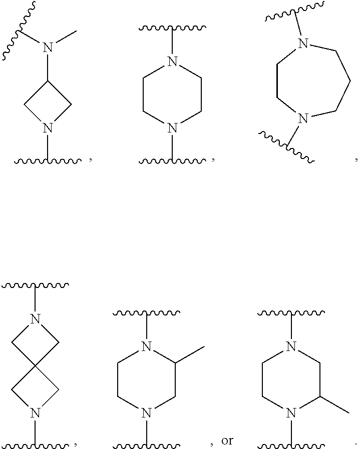

In more preferred embodiments, Z is selected from

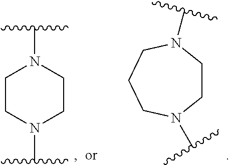 [SMILES: *N1CCN(*)CC1 ; *N1CCCN(*)CC1]

In most preferred embodiments, Z is

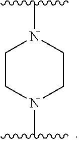 [SMILES: *N1CCN(*)CC1]

In preferred embodiments, the compound has the formula II

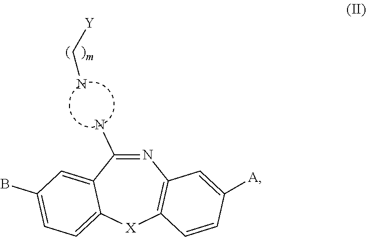

wherein B, X, A, Y, m and Z are defined as in any of the embodiments herein.

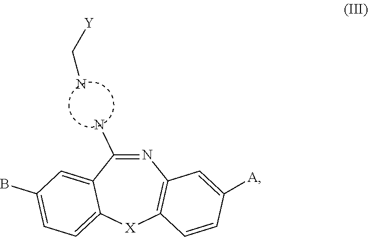

embodiments, the compound has the formula III wherein B, X, A, Y, and Z are defined as in any of the embodiments herein.

In preferred embodiments, B is selected from —Cl or —CF₃. In particularly preferred embodiments, B is —Cl. In particularly preferred embodiments, B is —CF₃. In preferred embodiments, B is defined as in any other embodiment herein, with the exception that B is not —CF₃. In preferred embodiments, B is defined as in any other embodiment herein, with the exception that B is not-Cl.

In preferred embodiments, A is selected from —H or —F. In most preferred embodiments, A is H.

In most preferred embodiments, X is —O—. In preferred embodiments of the invention,
A is one or two substituents independently selected from —H, —F, —Cl, —Br, —I, —CF₃, —OCF₃, —CF₂H, and —OCF₂H;
B is one or two substituents independently selected from —F, —Cl, —Br, —I, —CF₃, —OCF₃, —CF₂H, and —OCF₂H;
X is selected from —S-and-O—;
m is 1;
Y is selected from:

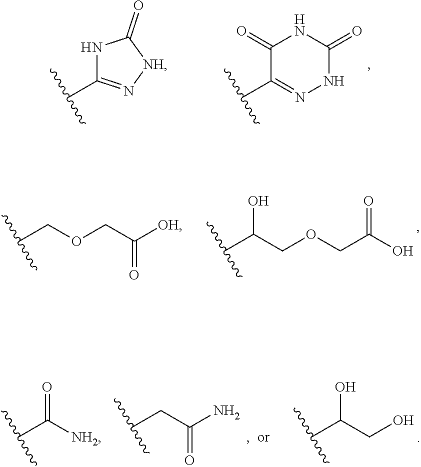

and
Z is selected from:

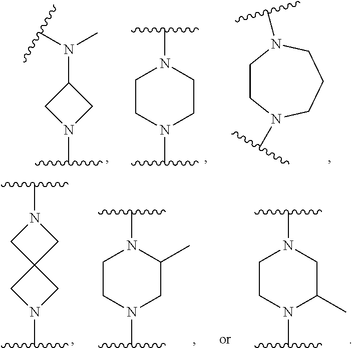

In preferred embodiments, the compound has the

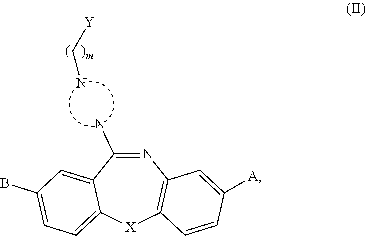

wherein B, X, A, Y, m and Z are defined as in any of the embodiments herein.

In most preferred embodiments, the compound has the formula III

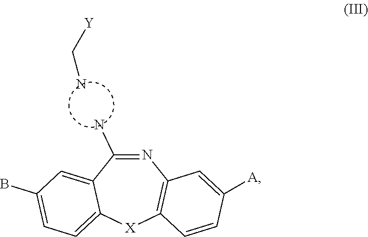

wherein B, X, A, Y, and Z are defined as in any of the embodiments herein.

In preferred embodiments of the invention, the compound has the formula (III), and A is selected from —H or —F; and B is selected from —Cl or —CF₃. In more preferred embodiments of the invention, the compound has the formula (III); A is selected from —H or —F; B is selected from —Cl or —CF₃; and X is —O—. In more preferred embodiments of the invention, the compound has the formula (III); A is selected from —H or —F; B is selected from —Cl or —CF₃; X is —O— and Z is

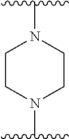 [SMILES: *N1CCN(*)CC1]

In more preferred embodiments of the invention, the compound has the formula (III); A is selected from —H or —F; B is selected from —Cl or —CF₃; X is —O— and Y is selected from

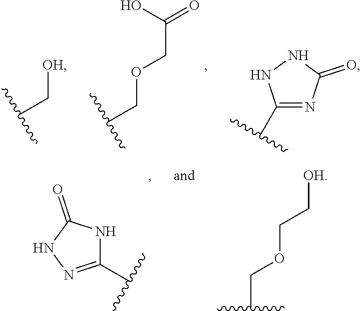

In more preferred embodiments of the invention, the compound has the formula (III); A is selected from —H or —F; B is selected from —Cl or —CF₃; X is —O—; Z is

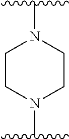 [SMILES: *N1CCN(*)CC1]

and Y is selected from

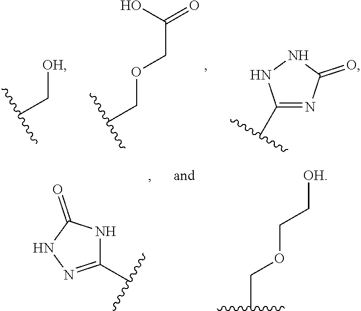

In preferred embodiments, the compound has the formula (III), and A is —H and B is —Cl. In preferred embodiments, the compound has the formula (III); A is —H; B is —Cl and X is —O—. In even more preferred embodiments, the compound has the formula (III); and A is —H; B is —Cl; X is —O— and Z is

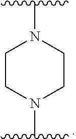 [SMILES: *N1CCN(*)CC1]

In preferred embodiments, the compound has the formula III; A is —H; B is —Cl; X is —O—, and Y is selected from

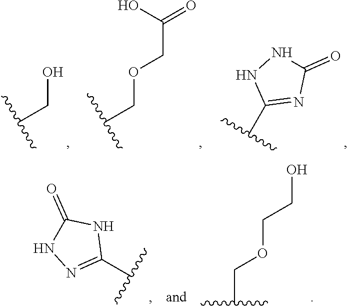

In even more preferred embodiments, the compound has the formula III; A is —H; B is —Cl; X is —O—; Z is

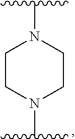 [SMILES: *N1CCN(*)CC1]

and Y is selected from

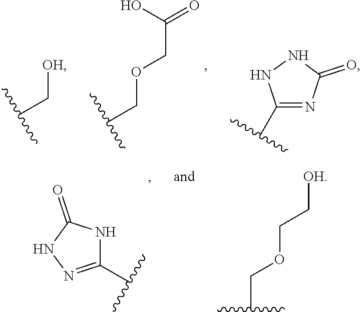

In preferred embodiments of the invention, the compound has the formula (II), and
(i) A is selected from —H or —F,
(ii) B is selected from —Cl or —CF₃,
(iii) m is 1,
(iv) Y is selected from the group consisting of

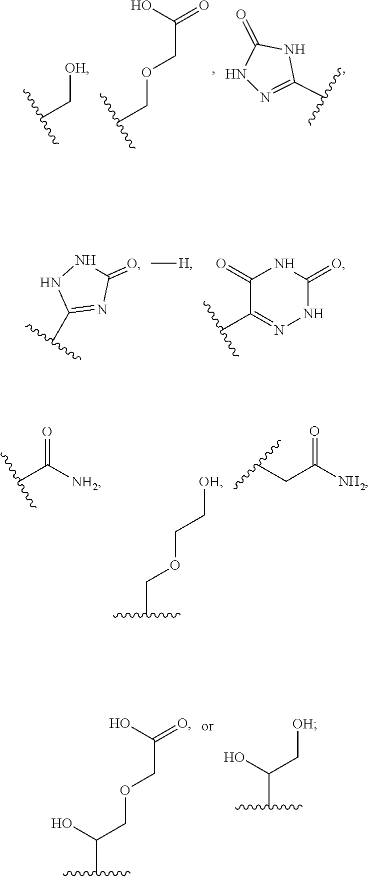

Z is selected from the group consisting of

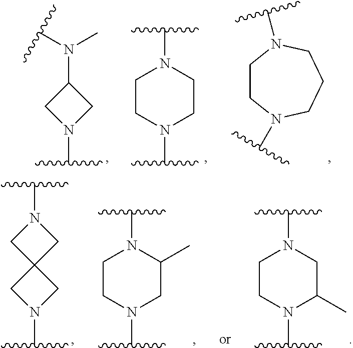

and preferably X is —O—.

In the context of the invention, the compounds are referred to with following numbering:

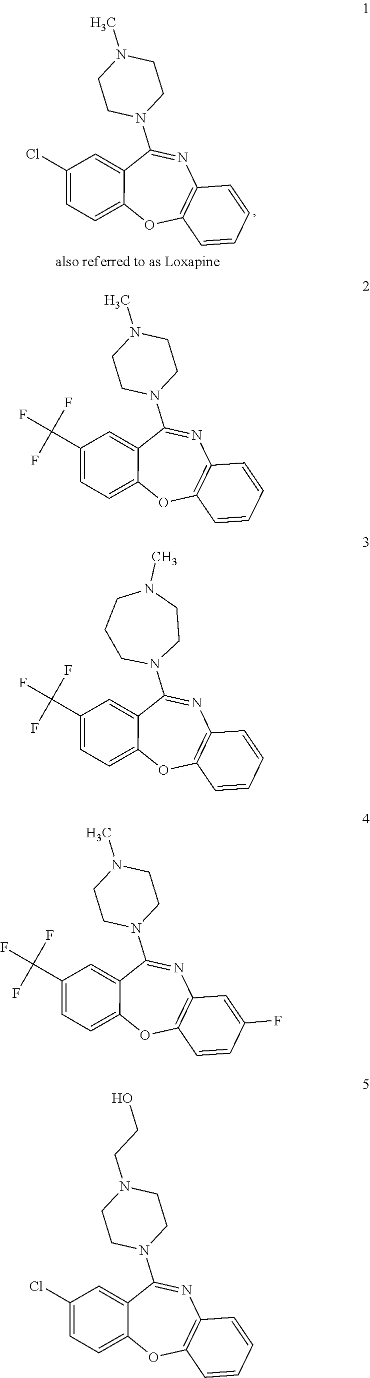 [SMILES: CN1CCN(C2=Nc3ccccc3Oc3ccc(Cl)cc32)CC1 ; CN1CCN(C2=Nc3ccccc3Oc3ccc(C(F)(F)F)cc32)CC1 ; CN1CCCN(C2=Nc3ccccc3Oc3ccc(C(F)(F)F)cc32)CC1 ; CN1CCN(C2=Nc3cc(F)ccc3Oc3ccc(C(F)(F)F)cc32)CC1 ; OCCN1CCN(C2=Nc3ccccc3Oc3ccc(Cl)cc32)CC1]

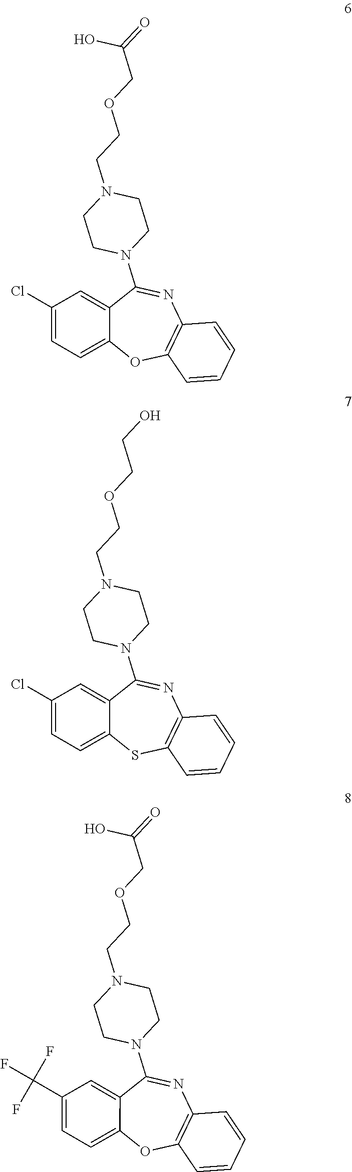 [SMILES: O=C(O)COCCN1CCN(C2=Nc3ccccc3Oc3ccc(Cl)cc32)CC1 ; OCCOCCN1CCN(C2=Nc3ccccc3Sc3ccc(Cl)cc32)CC1 ; O=C(O)COCCN1CCN(C2=Nc3ccccc3Oc3ccc(C(F)(F)F)cc32)CC1]

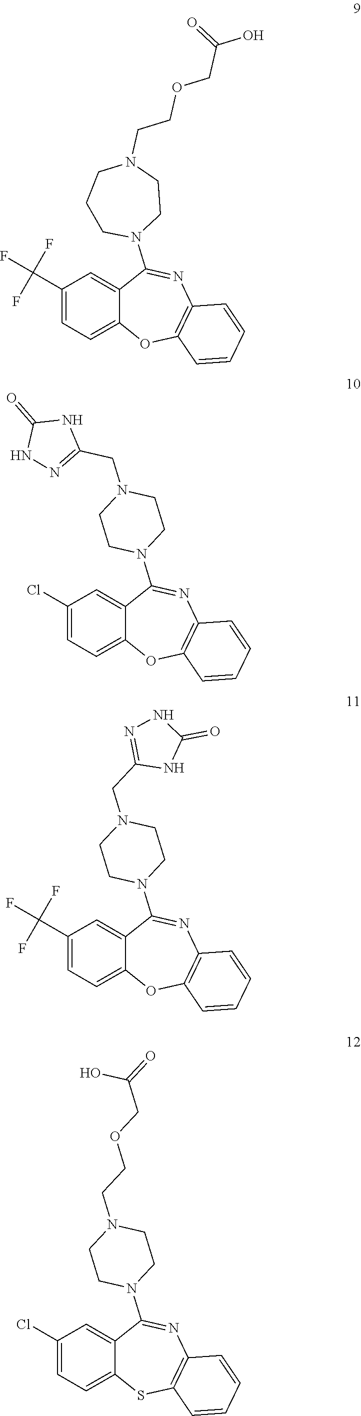 [SMILES: O=C(O)COCCN1CCCN(C2=Nc3ccccc3Oc3ccc(C(F)(F)F)cc32)CC1 ; O=c1[nH]nc(CN2CCN(C3=Nc4ccccc4Oc4ccc(Cl)cc43)CC2)[nH]1 ; O=c1[nH]nc(CN2CCN(C3=Nc4ccccc4Oc4ccc(C(F)(F)F)cc43)CC2)[nH]1 ; O=C(O)COCCN1CCN(C2=Nc3ccccc3Sc3ccc(Cl)cc32)CC1]

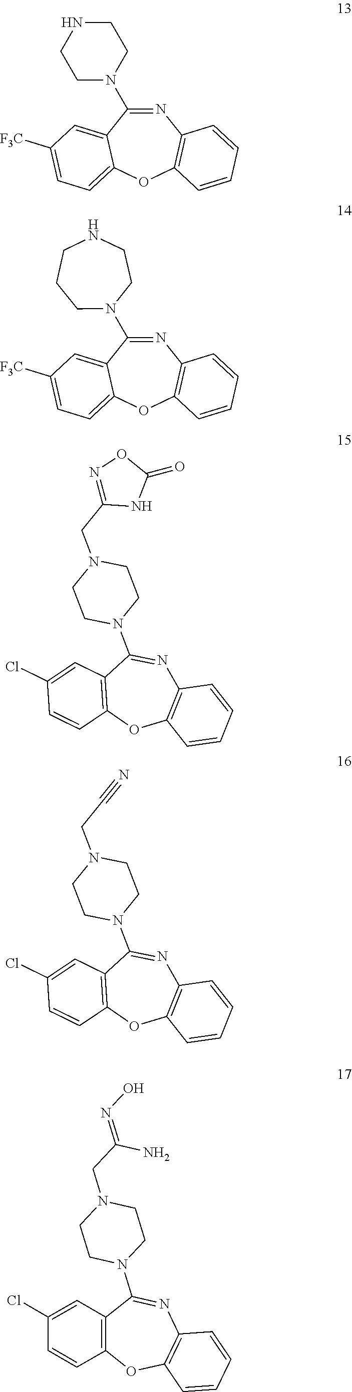

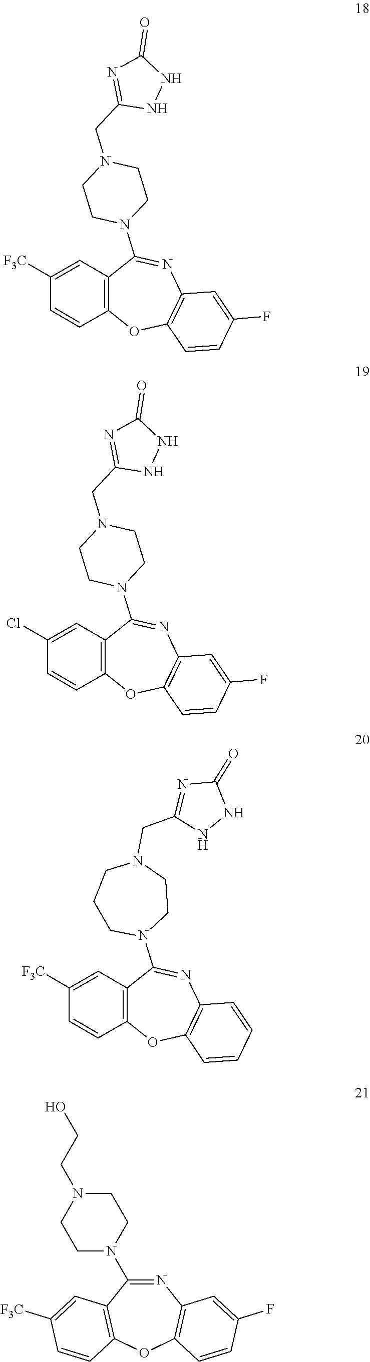

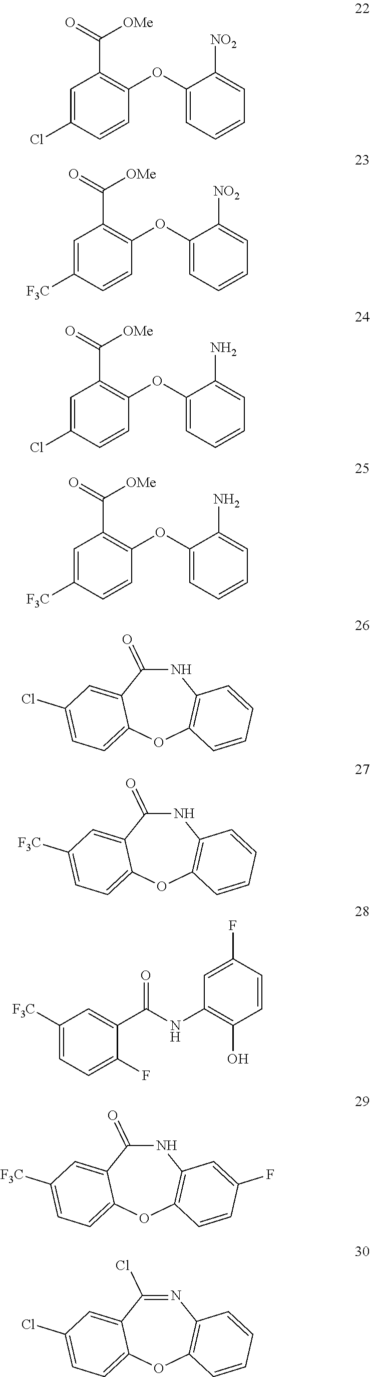

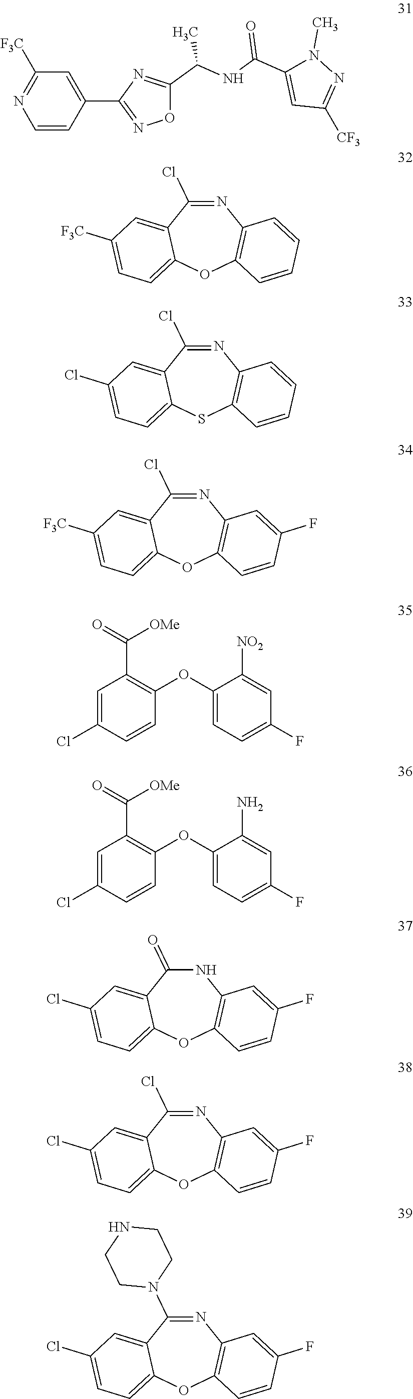

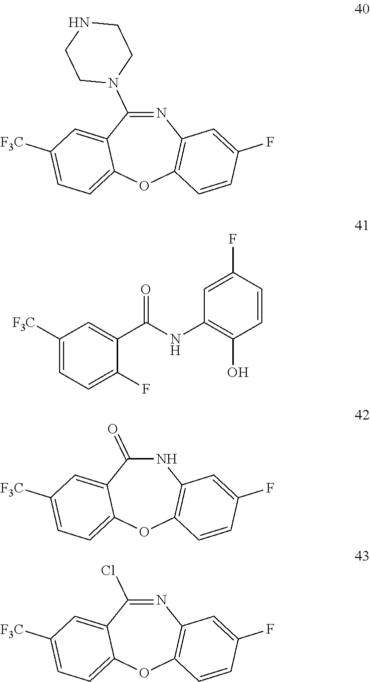

In particularly preferred embodiments, the compound is selected from the group consisting of compounds 1 to 21. Preferably, the compound is selected from the group consisting of compounds 2 to 21, preferably the compound is selected from the group consisting of compounds 2, 3, 4, 5, 6, 7, 8, 9, 10, 11, 12, 18, 19, 20, 21, more preferably the compound is selected from the group consisting of compounds 2, 3, 4, 5, 6, 7, 8, 9, and 10.

In particularly preferred embodiments, the compound is selected from the group consisting of compounds 6, 8, 9, 10, 11, 12, 15, 16, 17, 18, 19, 20 and 21, preferably the compound is selected from the group consisting of compounds 6, 8, 9, 10, 11, 12, 18, 19, 20, 21, more preferably the compound is selected from the group consisting of the compounds 6, 8, 9, and 10.

In particularly preferred embodiments, the compound is selected from the group consisting of compounds 6, 7, 8, 9, 10, 11, 15, 16, 17, 18, 19, 20 and 21, preferably the compound is selected from the group consisting of compounds 6, 7, 8, 9, 10, 11, 18, 19, 20 and 21, more preferably the compound is selected from the group consisting of compounds 6, 7, 8, 9, and 10.

In particularly preferred embodiments, the compound is selected from the group consisting of compounds 10, 11, 15, 18, 19 and 20. In particularly preferred embodiments, Y is

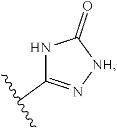 [SMILES: *c1n[nH]c(=O)[nH]1]

and optionally m is 1.

In preferred embodiments of the invention, the compounds are selected from the group consisting of compounds 3, 5-21; preferably the compounds are selected from the group consisting of compounds 3, 5-14; preferably the compounds are selected from the group of compounds 3, 5-11; more preferably the compounds are selected from the group of compounds 3, 5-10. In preferred embodiments, the compound is selected from the group of compounds 3, 5-10, 18-21. In preferred embodiments, the compound is any one of compounds 2 to 21.

In preferred embodiments of the invention, when X is —S—, then Y is not —(CH)₂—O—CH₂—CH₂—OH. In preferred embodiments of the invention, when X is —S—, B is —Cl, A is —H, m is 1, and Z is

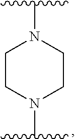 [SMILES: *N1CCN(*)CC1]

then Y is not —(CH)₂—O—CH₂—CH₂—OH.

In preferred embodiments of the invention, when m is 0, then Y is not-H. In preferred embodiments of the invention, when m is 1, then Y is not-H. In preferred embodiments of the invention, when m is 0, and optionally when B is further CI, then Y is not —(CH₂)—(CH₂)—OH. In preferred embodiments of the invention, when m is 1, and optionally when B is further CI, then Y is not —(CH₂)—OH.

In preferred embodiments of the invention, the compound is not a compound selected from the group of

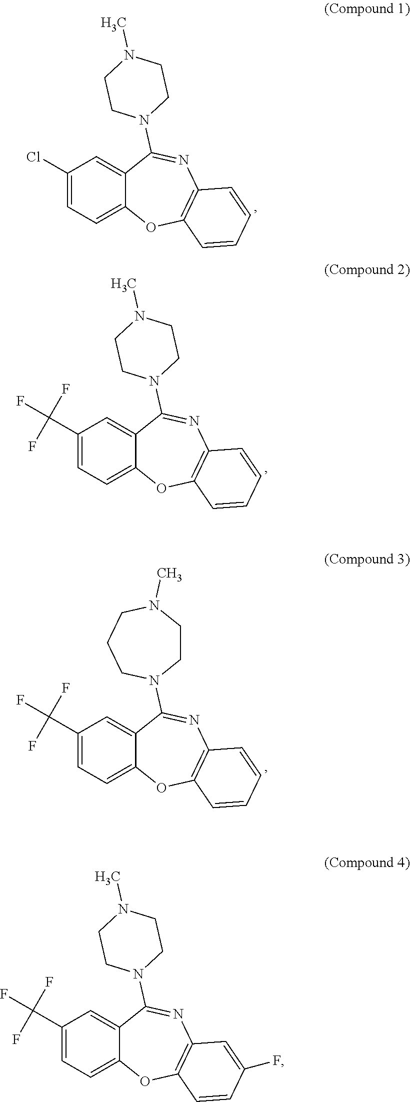 [SMILES: CN1CCN(C2=Nc3ccccc3Oc3ccc(Cl)cc32)CC1 ; CN1CCN(C2=Nc3ccccc3Oc3ccc(C(F)(F)F)cc32)CC1 ; CN1CCCN(C2=Nc3ccccc3Oc3ccc(C(F)(F)F)cc32)CC1 ; CN1CCN(C2=Nc3cc(F)ccc3Oc3ccc(C(F)(F)F)cc32)CC1]

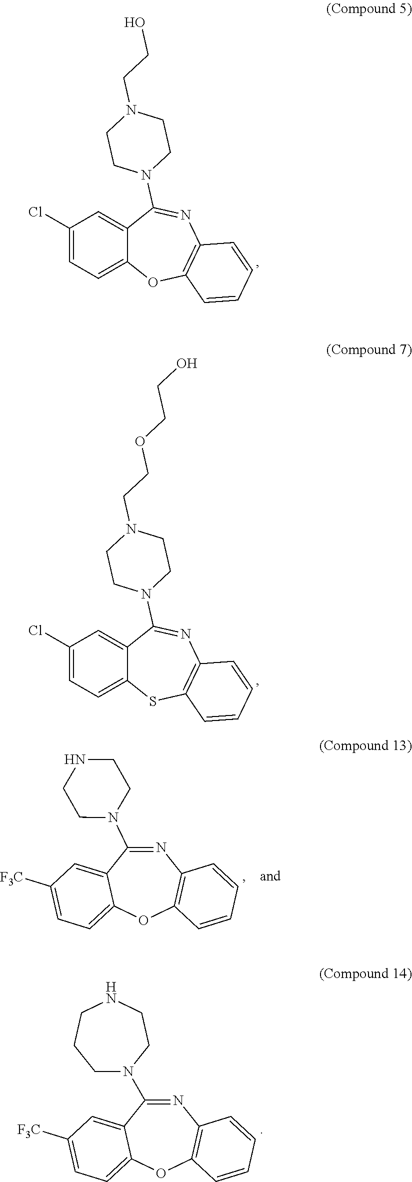 [SMILES: OCCN1CCN(C2=Nc3ccccc3Oc3ccc(Cl)cc32)CC1 ; OCCOCCN1CCN(C2=Nc3ccccc3Sc3ccc(Cl)cc32)CC1 ; FC(F)(F)c1ccc2c(c1)C(N1CCNCC1)=Nc1ccccc1O2 ; FC(F)(F)c1ccc2c(c1)C(N1CCCNCC1)=Nc1ccccc1O2]

In preferred embodiments, the compound is not

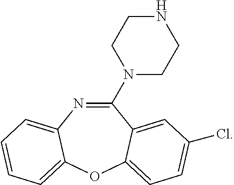 [SMILES: Clc1ccc2c(c1)C(N1CCNCC1)=Nc1ccccc1O2]

In preferred embodiments, the compound has an activity as an activator of a potassium channel in a cell, preferably of a potassium channel Slack (K_{Na}1.1). In the context of the invention, the term “Slack” or “Slack channel” refers to a class of sodium-activated potassium channels. These potassium channels are predominantly expressed in neuronal tissue where they are involved in neuronal excitability, burst firing and adaptation of firing rate. Preferably, the Slack channel is preferably encoded by the gene Kcnt1. Preferably, the slack channel refers to any isoform of the Potassium channel subfamily T member 1 disclosed in the UniProt entry Q5JUK3 and Q6ZPR⁴ (at the date of Feb. 2, 2023).

In preferred embodiments, the compound activates the potassium channel with an EC₅₀ of less than 100 μM, preferable of between 0.5 and 80 μM. In this context, the EC₅₀ preferably refers to an EC₅₀ that is determined in an Na⁺ free buffer with 2 mM Ca²⁺, 2 mM Mg²⁺ and 0.03% DMSO.

In preferred embodiments, the compound, which when administered to an animal does not penetrate the blood brain barrier (BBB), or which penetrates the BBB less than a reference compound, such as Loxapine.

In preferred embodiments, the compound does not bind the human dopamine receptor, more preferably wherein the compound binds the human dopamine receptor less compared to a reference compound, such as Loxapine.

In preferred embodiments of the invention, the compound binds to an off-target selected from the group consisting of alpha-1A adrenergic receptor (α_{1A}), dopamine receptor D₁ (D₁), the short form of dopamine receptor D₂ (D_{2S} or D_{2Sh}), histamine H1 receptor (H₁), a serotonin receptor (preferably 5-HT_{2A} and 5-HT_{2B}), norepinephrine transporter (NET), serotonin transporter (SET), cannabinoid receptor 1 (CB1). In preferred embodiments of the invention, the compound binds to a target selected from the group consisting of alpha-1A adrenergic receptor (α_{1A}), dopamine receptor D₁ (D₁), the short form of dopamine receptor D₂ (D_{2S} or D_{2Sh}), histamine H1 receptor (H₁), a serotonin receptor (preferably 5-HT_{2A} and 5-HT_{2B}), norepinephrine transporter (NET), and serotonin transporter (SET). In preferred embodiments of the invention, the compound binds to a target selected from the group consisting of dopamine receptor D₁ (D₁), Histamine H1 receptor (H₁), a serotonin receptor (preferably 5-HT_{2A} and 5-HT_{2B}). By binding to such an off-target, the antipruritic effect of the compounds of the invention may be further increased. In preferred embodiments, the compound binds to a H₁ receptor (preferably, wherein the compound of the invention is an antagonist of H₁ receptor). In further preferred embodiments, the compound of the invention binds to a 5-HT receptor, more preferably a 5-HT₂ receptor, in particular a 5-HT_{2A} receptor, preferably, wherein the compound is an agonist of the 5-HT receptor, 5-HT₂ receptor, and/or 5-HT₂A receptor.

In preferred embodiments of the invention, the compound is Compound 6, Compound 8 Compound 10 or Compound 11. In preferred embodiments of the invention, the compound is Compound 6, Compound 8 or Compound 10. In more preferred embodiments of the invention, the compound is Compound 6 or Compound 10.

In another preferred embodiment of the invention, the compound is Compound 6 or Compound 21. In more preferred embodiment of the invention, the compound is Compound 21.

In preferred embodiments of the invention, the compound is isolated. In preferred embodiments, the compound is dissolved in a solvent or a dispersion. In preferred embodiments, the compound has a purity of at least 75%, optionally at least 90%, optionally at least 95%. In this context, the purity refers to the absence of any foreign material (in particular chemical substances) which may be present in a composition comprising the compound of the invention. Impurities may occur naturally, may be added or generated during the synthesis and/or purification of the compound of the invention. For example, impurities include a starting material, a solvent, an intermediate or a reactant, a degradation product of any of the foregoing or of a desired compound, leftovers of protecting groups after deprotection, and combinations thereof.

The compounds of the present invention may exist in tautomeric forms. Preferably, the depiction of a single tautomer, for example in a functional group or a compound, is understood to represent the compound or group in all its tautomeric forms. For example, if not indicated otherwise, the compounds 10, 11, 15, 17, 18, 19, and/or 20 preferably each refer to both the following tautomeric forms:

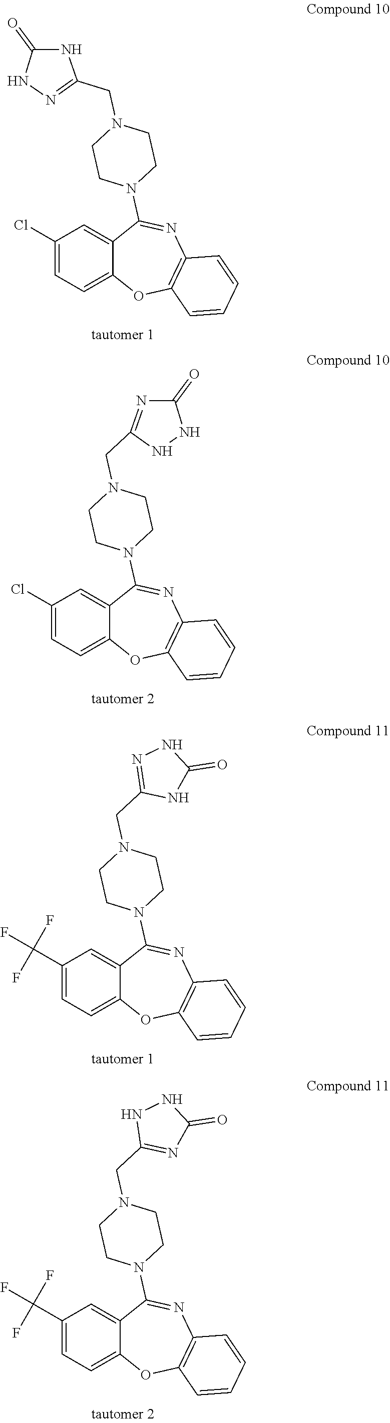 [SMILES: O=c1[nH]nc(CN2CCN(C3=Nc4ccccc4Oc4ccc(Cl)cc43)CC2)[nH]1 ; O=c1nc(CN2CCN(C3=Nc4ccccc4Oc4ccc(Cl)cc43)CC2)[nH][nH]1 ; O=c1[nH]nc(CN2CCN(C3=Nc4ccccc4Oc4ccc(C(F)(F)F)cc43)CC2)[nH]1 ; O=c1nc(CN2CCN(C3=Nc4ccccc4Oc4ccc(C(F)(F)F)cc43)CC2)[nH][nH]1]

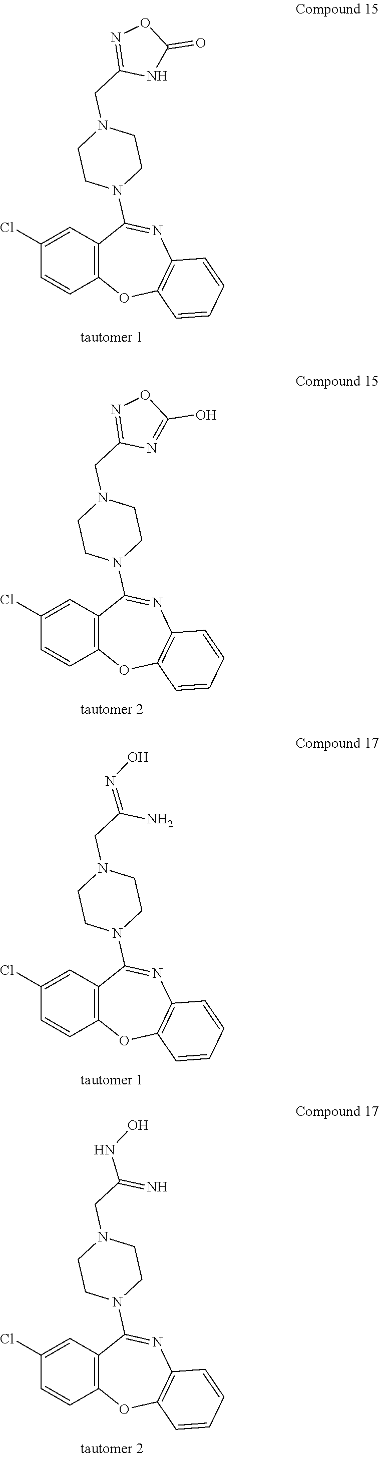 [SMILES: O=c1[nH]c(CN2CCN(C3=Nc4ccccc4Oc4ccc(Cl)cc43)CC2)no1 ; Oc1nc(CN2CCN(C3=Nc4ccccc4Oc4ccc(Cl)cc43)CC2)no1 ; N/C(CN1CCN(C2=Nc3ccccc3Oc3ccc(Cl)cc32)CC1)=N\O ; N=C(CN1CCN(C2=Nc3ccccc3Oc3ccc(Cl)cc32)CC1)NO]

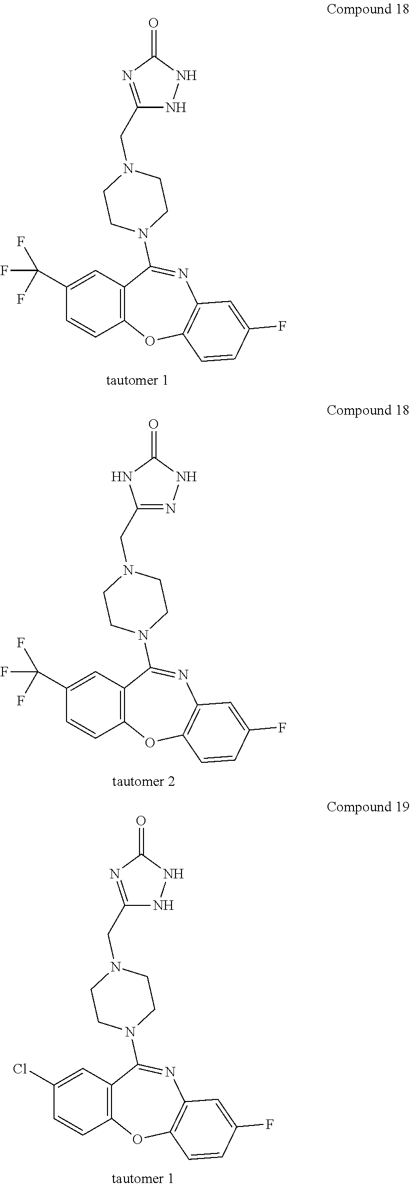 [SMILES: O=c1nc(CN2CCN(C3=Nc4cc(F)ccc4Oc4ccc(C(F)(F)F)cc43)CC2)[nH][nH]1 ; O=c1[nH]nc(CN2CCN(C3=Nc4cc(F)ccc4Oc4ccc(C(F)(F)F)cc43)CC2)[nH]1 ; O=c1nc(CN2CCN(C3=Nc4cc(F)ccc4Oc4ccc(Cl)cc43)CC2)[nH][nH]1]

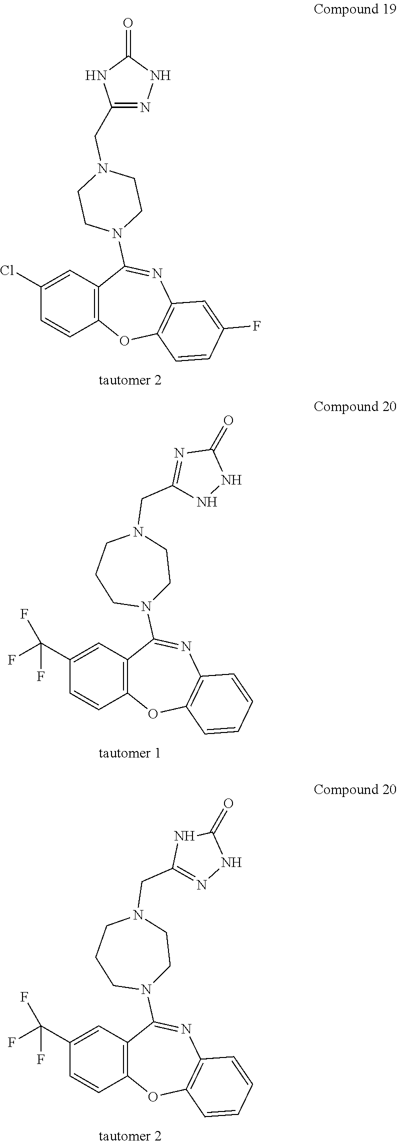 [SMILES: O=c1[nH]nc(CN2CCN(C3=Nc4cc(F)ccc4Oc4ccc(Cl)cc43)CC2)[nH]1 ; O=c1nc(CN2CCCN(C3=Nc4ccccc4Oc4ccc(C(F)(F)F)cc43)CC2)[nH][nH]1 ; O=c1[nH]nc(CN2CCCN(C3=Nc4ccccc4Oc4ccc(C(F)(F)F)cc43)CC2)[nH]1]

Preferably, Compound 10 is Compound 10 tautomer 1 or Compound 10 tautomer 2. Preferably, Compound 11 is Compound 11 tautomer 1 or Compound 11 tautomer 2. Preferably, Compound 15 is Compound 15 tautomer 1 or Compound 15 tautomer 2. Preferably, Compound 17 is Compound 17 tautomer 1 or Compound 17 tautomer 2. Preferably, Compound 18 is Compound 18 tautomer 1 or Compound 18 tautomer 2. Preferably, Compound 19 is Compound 19 tautomer 1 or Compound 19 tautomer 2. Preferably, Compound 20 is Compound 20 tautomer 1 or Compound 20 tautomer 2.

In a second aspect, the invention pertains to a pharmaceutical composition comprising the compound of any one of the aspects herein, or a salt, solvate, or ester thereof, and a pharmaceutically acceptable carrier or excipient.

Preferably, the pharmaceutically acceptable composition comprises the compound of any aspect of the invention, wherein the compound is in the form of a prodrug, a salt, a racemic mixture, a crystalline from, a polymorph, a solvate or combinations thereof.

The term “pharmaceutically acceptable carrier or excipient” means a carrier or excipient that is useful in preparing a pharmaceutical composition that is generally safe, non-toxic and neither biologically nor otherwise undesirable, and includes excipients that are acceptable for veterinary use as well as human pharmaceutical use. It includes solvents, emulsifiers, suspending agents, disintegrators, binding agents, stabilizing agents, diluents, gelling agents, preservatives, lubricants, surfactants and other similar carriers. Such a pharmaceutically carrier or excipient must be “acceptable” in the sense of being compatible with the other ingredients of a subject composition and not injurious to the patient.

In preferred embodiments of the pharmaceutical composition, the compound, or a salt, solvate, or ester thereof, is present in an amount of 0.1% w/w to 10% w/w, optionally in an amount of 1% w/w to 10% w/w. In preferred embodiments, the salt of the compound is a hydrochloride salt of the compound.

In preferred embodiments of the pharmaceutical composition, the composition is an oral composition, a topical composition, or an infusion. In preferred embodiments of the pharmaceutical composition, the composition is an oral composition, a topical composition, or a parenteral composition. In preferred embodiments, the composition is in the form of a patch, suppository, syringe, injector pen, spray bottle, mask, pastille and/or implant inhaler, nebulizer, creme and/or vaporizer. In preferred embodiments of the pharmaceutical composition is a unit dosage form. In particular preferred embodiments, the composition is in the form of a creme.

In preferred embodiments of the composition, the composition the dosage form is an extended-release dosage form. Preferably the extended release dosage form releases the compound over the course of 5 h, 10 h, 18 h, 24 h, 36 h, 48 h, 72 h, 1 week, 2 weeks, 1 month, 6 months, 12 months and/or 2 years. In this context, “over the course” encompasses a release, wherein the compound is continuously, periodically/or non-periodically released to the body of the subject. Preferably, the extended release dosage form is an implant. Preferably, the implant comprises the compound of the first aspect of the invention.

The invention furthermore pertains to the following special preferred embodiments. These special preferred embodiments can be combined with any of the aspects, embodiments, and claims herein.

In special preferred embodiments preferred embodiments, Y is selected from a linear —C₁₋₃alkyl, wherein the terminal —CH₃ of the linear —C₁₋₃alkyl is monosubstituted with —OR¹, wherein R¹ is —(CH₂)—COOH. More preferably, Y is selected from a linear —C₁₋₂alkyl, wherein the terminal —CH₃ of the linear —C₁₋₂alkyl is monosubstituted with —OR¹, wherein R¹ is —(CH₂)—COOH. Most preferably, Y is a methyl group that is monosubstituted with —OR¹, wherein R¹ is —(CH₂)—COOH.

In special preferred embodiments, m=1 and Y is selected from a linear —C₁₋₃alkyl, wherein the terminal —CH₃ of the linear —C₁₋₃alkyl is monosubstituted with —OR¹, wherein R¹ is —(CH₂)—COOH. More preferably, m=1 and Y is selected from a linear —C₁₋₂alkyl, wherein the terminal-CH₃ of the linear —C₁₋₂alkyl is monosubstituted with —OR¹, wherein R¹ is —(CH₂)—COOH. Most preferably, m=1 and Y is a methyl group that is monosubstituted with —OR¹, wherein R¹ is —(CH₂)—COOH.

In special preferred embodiments, when referring to Y, the 5- to 6-membered unsaturated heterocycle comprising 3 hetero atoms that are selected from O and N is
a 5-membered unsaturated heterocycle comprising 3 hetero atoms that are N, wherein the 5-membered unsaturated heterocycle is monosubstituted with ═S or ═O, preferably with ═O; or
a 6-membered heterocycle comprising 3 hetero atoms that are nitrogen, wherein the 6-membered heterocycle is di-substituted with ═O or ═S, preferably with ═O.

In preferred embodiments, when referring to Y, the 5- to 6-membered unsaturated heterocycle comprising 3 hetero atoms that are selected from O and N is a 5-membered heterocycle comprising 3 hetero atoms that are nitrogen, wherein the 5-membered heterocycle is monosubstituted with ═O.

In a special aspect, the invention pertains to a compound or composition for use in the treatment of a condition in a subject, the composition comprising an effective amount of the compound of the first aspect of the invention, or a salt, solvate, or ester thereof.

In a third aspect, the invention pertains to a compound or composition for use in the treatment of a condition in a subject, the composition comprising an effective amount of the compound of the first aspect of the invention, or a salt, solvate, or ester thereof, wherein the condition is treatable by activating a potassium channel in a cell associated with the pathology of the condition, the treatment comprising administering the compound or composition to the subject in need thereof.

Preferably, the subject of the invention is an animal. In this context, the term “animal” includes humans. Preferably, the animal is a mammal or bird, most preferably a mammal. Preferably, the mammal is a human. Preferably, the mammal is a non-human primates, dog, cat, sheep, cattle, goat, pig, mouse, rat, rabbit or guinea pig.

In this context the term “treatment” includes the application or administration of a therapeutic agent (such as a compound of the invention) or procedure to a patient in need thereof, purpose to cure, heal, alleviate, relieve, alter, remedy, ameliorate, improve, affect or prevent the condition, the symptoms of the condition, or the predisposition toward the condition. Hence, the term “treatment” can include prophylactic treatment of a condition the symptoms of a condition.

Preferably, the application or administration comprises intravenous, intraperitoneal, subcutaneous, intramuscular, intrathecal, peridural, topical, oral, gastric and/or rectal administration. In preferred embodiments, the application or administration comprises intrathecal, and/or peridural, administration. In preferred embodiments, the application or administration comprises intravenous, intraperitoneal, subcutaneous, intramuscular, topical, oral, gastric and/or rectal administration. CNS administration, in particular intrathecal or peridural administration, is likely to result in a more potent analgesic effect in case of neuropathic pain compared to other types of administration such as than oral or i.p. administration.

In preferred embodiments, the condition is a pruritic condition, such as an acute or chronic pruritus. In preferred embodiments, the pruritic condition is associated with a second pathology, such as one selected from:
a dermatological disorder such as xerosis or xeroderma (dry skin), dermatitis or eczema (e.g., atopic dermatitis), psoriasis (e.g., plaque psoriasis), prurigo (e.g., prurigo nodularis), urticaria (e.g., chronic idiopathic urticaria), a connective tissue disorder (e.g., dermatomyositis), post-burn pruritus;
a kidney disorder (e.g., chronic kidney disease, chronic kidney failure or end-stage renal disease), dialysis (e.g., hemodialysis), uremic pruritus;
a hepato-biliary disorder (e.g., cholestasis, primary biliary cholangitis, primary sclerosing cholangitis, secondary sclerosing cholangitis, hepatitis, toxic liver disease, chronic liver disease or cirrhosis), cholestatic pruritus;
an endocrine disorder (e.g., hyperthyroidism or diabetes mellitus);
a metabolic disorder (e.g., iron deficiency or iron overload);
a benign or malignant neoplasm (e.g., a solid tumor, a carcinoma or a hematological neoplasm [e.g., Hodgkin's lymphoma, non-Hodgkin's lymphoma, myeloproliferative disease, or polycythemia vera]);
an infectious disease (e.g., a viral infection such as an infection with herpes simplex, herpes zoster, varicella, human immunodeficiency virus (HIV), hepatitis; a bacterial infection or a parasitosis);
a neurological disorder (e.g., degenerative neurological diseases, multiple sclerosis, brain tumors, postherpetic neuralgia, small-fiber neuropathies, brachioradial pruritus or notalgia paresthetica), neuropathic itch, neurogenic itch;
a psychiatric disease (e.g., depression, obsessive compulsive disorder, delusional disorder, eating disorder, or anxiety);
drug-induced pruritus (e.g., by opioids, antibiotics, antimalarial agents, ACE inhibitors, angiotensin receptor antagonists, anti-arrhythmic agents, antidepressants, antidiabetic drugs, antihypertensive drugs, anticonvulsants, anti-inflammatory drugs, betablockers, bronchodilators, calcium antagonists, diuretics, hormones, immunosuppressive drugs, antilipids, neuroleptics, plasma expanders, tranquilizers, or uricostatics);
pruritus in the elderly;
pruritus in pregnancy; or/and
chronic idiopathic pruritus.

In preferred embodiments of the invention, the pruritic condition is associated with second pathology, such as one selected from:
a dermatological disorder such as xerosis or xeroderma (dry skin), dermatitis or eczema (e.g., atopic dermatitis), psoriasis (e.g., plaque psoriasis), prurigo (e.g., prurigo nodularis), urticaria (e.g., chronic idiopathic urticaria), a connective tissue disorder (e.g., dermatomyositis), post-burn pruritus;
a kidney disorder (e.g., chronic kidney disease, chronic kidney failure or end-stage renal disease), dialysis (e.g., hemodialysis), uremic pruritus;
a hepato-biliary disorder (e.g., cholestasis, primary biliary cholangitis, hepatitis, chronic liver disease or cirrhosis), cholestatic pruritus;
a benign or malignant neoplasm (e.g., a solid tumor, a carcinoma or a hematological neoplasm [e.g., Hodgkin's lymphoma, non-Hodgkin's lymphoma or polycythemia vera]);
neuropathic itch (e.g., brachioradial pruritus or notalgia paresthetica), neurogenic itch:
drug-induced pruritus (e.g., by opioids),
pruritus in the elderly, or/and
chronic idiopathic pruritus.

In preferred embodiments, the myeloproliferative disease is a myeloproliferative neoplasm.

In preferred embodiments, the composition for use is the pharmaceutical composition of the second aspect of the invention. Additionally, the treatment may comprise applying or administering further antipruritic drugs before, simultaneously or after the application or administration of the compound or composition of the invention. Preferably, the further antipruritic drug is selected from a systemic, oral and/or topical drug. Preferably, the further antipruritic drug is selected from the group of corticosteroids, antihistamines, anesthetics, phosphodiesterase-4 inhibitors, capsaicin, μ-opioid receptor antagonists, antidepressants, immunosuppressants, anticonvulsants, janus kinase inhibitors, K-opioid receptor agonist and Thalidomide- and/or Butorphanol-based drugs.

Preferably, the treatment according to the present invention is conducted before, simultaneously or after further antipruritic treatments, preferably wherein the further antipruritic treatment is selected from the group of applying or administering aluminum triacetate solution, olive oil, jewelweed, calamine lotion, sodium bicarbonate paste, ammonium hydroxide and/or papain-based topical creams, cannabis, pigweed (portulaca oleracaea), ashoka (sarco asoca or saraca asoca), fig (fificus carica) and/or cannabinoids.

In preferred embodiments, the condition is pain or a pain related noxious sensation in the subject.

In preferred embodiments, the pain or a pain related noxious sensation is selected from
neuropathic pain induced by traumatic nerve injury, cancer and cancer treatments (e.g., chemotherapy), neurological conditions (e.g., multiple sclerosis), neurodegenerative conditions (e.g., Parkinson's disease), trigeminal neuralgia, diabetic peripheral neuropathy, stroke, shingles, HIV, Hansen's disease (leprosy), Guillain-Barre syndrome, blood vessel disease, vascular malformations, autoimmune conditions,
acute post-operative pain,
inflammatory pain, rheumatoid arthritis, osteoarthritis, and/or
nociplastic pain.

In preferred embodiments, the composition comprises at least one additional therapeutically active and/or pain reducing agent. Preferably, the at least one additional therapeutic agent is an agent for the treatment of the second pathology as mentioned herein.

In a fourth aspect, the invention pertains to a method of synthesizing a compound of the first aspect of the invention.

In this context the method of synthesizing a compound may include one or more synthesis steps. Preferably, the synthesis step is a chemical reaction step and/or a purification step.

In particular, the method of providing a compound can comprise any of the synthesis steps disclosed herein and/or synthesis steps analogous thereto, which are readily known and available to one of ordinary skill in the art of organic synthesis.

In particular, the method of providing a compound can comprise any of the chemical reaction steps disclosed herein and/or chemical reaction steps analogous thereto, which are readily known and available to one of ordinary skill in the art of organic synthesis.

In particular, the method of providing a compound can comprise any of the purification steps disclosed herein and/or purification steps analogous thereto, which are readily known and available to one of ordinary skill in the art of organic synthesis.

Preferably, the chemical reaction step is followed or preceded by a purification step.

Purification in this context refers to any suitable method for removing an impurity fraction. Such purification methods are well known and include e.g., column chromatography, selective precipitation, trituration and elution of impurities with a suitable solvent in which the desired compound is not soluble, etc. The fraction of impurities removed may be such that the compound is prepared in substantially pure form; that is, for example, in a percentage purity as described above. In other embodiments, the compound of the invention is provided by said method in an impure form.

The method of synthesizing a compound according to the invention may be started, interrupted and continued at any point. Preferably, the method of synthesizing a compound according to the invention is interrupted after a chemical reaction step or a purification step. If possible, the method includes the isolation of by-products or intermediates of the chemical reaction steps of the invention.

A synthesis step, such as a chemical reaction step or a purification step, according to the invention may be conducted using any suitable solvent. For example, the choice of a solvent may depend on the stability of the products, the polarity of the solvent and/or the compound, the boiling point of the solvent, the acidity of the solvent, if a solvent is protic/aprotic and/or the biocompatibility of the solvent. Preferred solvents include aliphatic alcohols (such as methanol, ethanol, n-propanol, isopropanol), acetone, acetonitrile, ether, N,N-dimethylformamide (DMF), tetrahydrofuran (THF), dichloromethane, water or a mixture of two or more of these liquids. Preferably, a synthesis step according to the invention may be carried out under an inert gas such as nitrogen, helium, neon, argon, krypton, xenon, radon and/or sulphur hexafluoride. Moreover, the solvent, which is used in the synthesis step, for example in the chemical reaction step or the purification step, may be cleansed using such a gas.

In preferred embodiments, the method of synthesizing a compound according to the invention comprises a synthesis step, preferably a chemical reaction step, of synthesizing a lactam core. Preferably, said lactam core comprises the structure

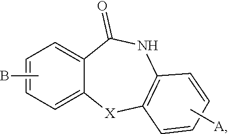

wherein A, X and B are defined as in any embodiment herein.

In preferred embodiments, the lactam core of the invention is synthesized according to Synthesis Route A or Synthesis Route B comprising the corresponding chemical reaction steps shown in Table 1. The individual chemical reaction steps in Synthesis Routes A and Synthesis Route B are preferably preformed consecutively, therefore in the order
Chemical Reaction Step 1a→Chemical Reaction Step 2a→Chemical Reaction Step 3a, or
Chemical Reaction Step 1b→Chemical Reaction Step 2b.

TABLE 1
Chemical Reaction Steps of Synthesis Route A and Synthesis Route B.
Synthesis Route A

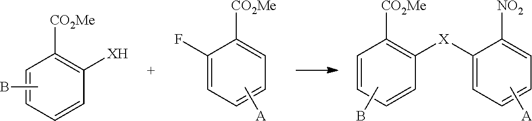

Chemical Reaction Step 1a

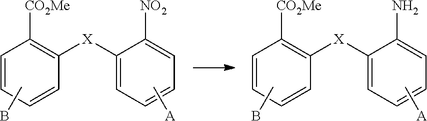

Chemical Reaction Step 2a

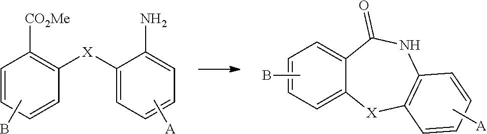

Chemical Reaction Step 3a
Synthesis Route B

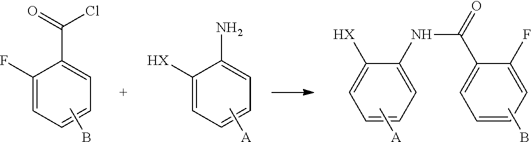

Chemical Reaction Step 1b

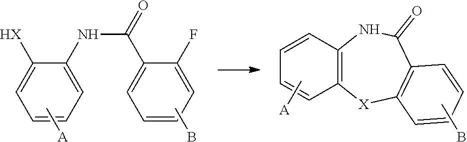

Chemical Reaction Step 2b

In further preferred embodiments, the method of synthesizing a compound according to the invention comprises a synthesis step, preferably a chemical reaction step, of converting a lactam core to an imidoyl chloride derivative thereof and/or converting the lactam core and/or the imidoyl chloride derivative thereof to a compound of the invention. Said converting a lactam core to an imidoyl chloride derivative thereof and/or converting the lactam core and/or the imidoyl chloride derivative thereof to a compound of the invention is preferably conducted using the Synthesis Route C with their corresponding Chemical Reaction steps shown in Table 2. Preferably, the individual Chemical Reaction steps in Synthesis Route C are performed consecutively, therefore in the order of
Chemical Reaction Step 1c→Chemical Reaction Step Reaction 2c.

TABLE 2
Chemical reaction steps of Synthesis Route C
Synthesis Route C

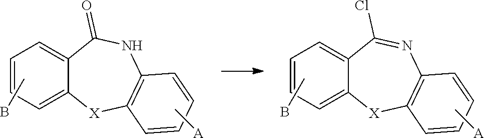

Chemical Reaction Step 1c

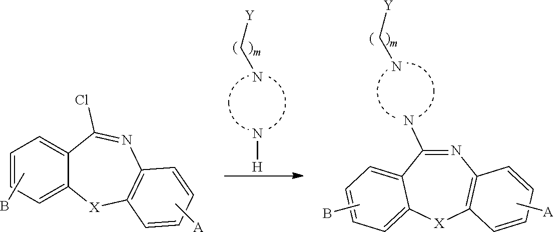

Chemical Reaction Step 2c, wherein

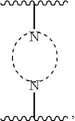

m, and Y are defined according to any other aspect of the invention.

In preferred embodiments, the method of synthesizing a compound according to the invention comprises conducting the Synthesis Route A followed by conducting the Synthesis Route C. In preferred embodiments, the method of synthesizing a compound according to the invention comprises conducting the Synthesis Route B followed by conducting the Synthesis Route C.

In preferred embodiments, Synthesis Route B comprises an additional Chemical Reaction Step 0b that is conducted before the Chemical Reaction Step 1b.

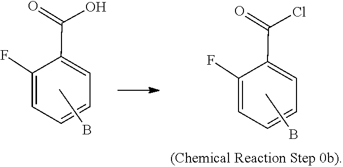

In preferred embodiments of the invention, when

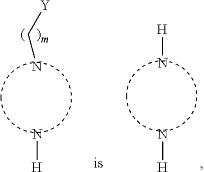

preferably when

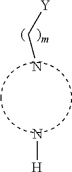

is piperazine or homopiperazine, the Synthesis Route C comprises a Chemical Reaction Step 3c after the Chemical Reaction Step 2c,

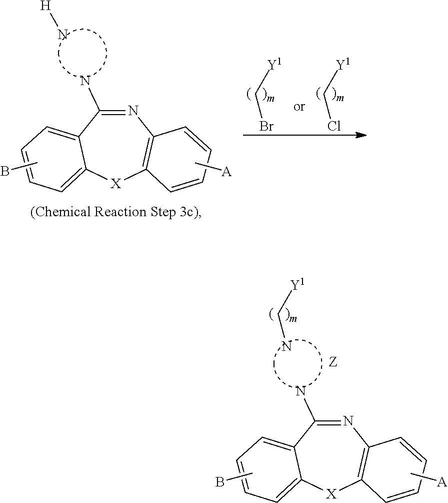

wherein Y¹ is defined as Y with the additional feature that Y¹ is not H.

In preferred embodiments, the method of synthesizing a compound according to the invention comprises starting the synthesis at any synthesis step, preferably at any chemical reaction step disclosed herein or at a purification step prior or after any chemical reaction step disclosed herein. In preferred embodiments, the method of synthesizing a compound according to the invention comprises conducting a reaction with a reaction intermediate or reaction product of a synthesis step, preferably a chemical reaction step, disclosed herein, preferably in the Synthesis Routes A, B and/or C.

In preferred embodiments, the method of synthesizing a compound according to the invention comprises conducting at least one synthesis step, preferably at least one chemical reaction step, disclosed herein, preferably in the Synthesis Routes A, B and/or C.

In preferred embodiments of the invention, the Chemical Reaction Step 1a is conducted in a solvent containing potassium carbonate. In preferred embodiments, Chemical Reaction Step 1a is conducted in N,N-dimethylformamide (DMF). In preferred embodiments, Chemical Reaction Step 1a is conducted at 100 to 140° C. most preferably at 120° C.

In preferred embodiments, Chemical Reaction Step 2a is conducted using a reduction agent, preferably wherein the reduction agent is SnCl₂·2H₂O, and optionally wherein the Chemical Reaction Step 2a is conducted in a mixture of ethanol/conc. HCl 1:1.

In preferred embodiments, the Chemical Reaction Step 3a comprises treatment of

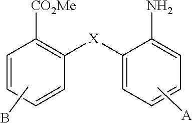

with an acid, more preferably wherein the acid is sulfuric acid, most preferably concentrated sulfuric acid. In preferred embodiments, the reaction is conducted in DMF. In preferred embodiments, Chemical Reaction Step 3a is conducted in DMF at 100 to 140° C., most preferably at 120° C.

In preferred embodiments the Chemical Reaction Step Ob comprises the reaction of addition of thionyl chloride to

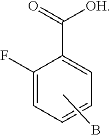

In preferred embodiments of the invention, the Chemical Reaction Step 1b comprises the dropwise addition of

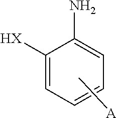

to a solution of

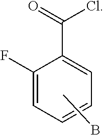

In preferred embodiments, the Chemical Reaction Step 1b is −20 to 50° C., more preferably from −10 to 30° C., most preferably at 0° C.

In preferred embodiments of the invention, the Chemical Reaction Step 2b is conducted in a basic solution, preferably wherein the basic solution comprises NaOH. In preferred embodiments, Chemical Reaction Step 2b is conducted in DMF. In preferred embodiments of the invention, Chemical Reaction Step 2b is conducted at 120 to 170° C., more preferably at 140 to 160° C., most preferably at 150° C.

In preferred embodiments, Chemical Reaction Step 1c is conducted in phosphorus oxychloride, more preferably under reflux, and optionally, wherein the phosphorus oxychloride comprises N,N-dimethylaniline.

In preferred embodiments of the invention, the Chemical Reaction Step 2c is conducted in p-xylene, optionally wherein the reaction is conducted at 110 to 150° C., preferably at 120 to 140° C.

In preferred embodiments of the invention, the Chemical Reaction Step 3c is conducted with an additional base in the reaction mixture, preferably wherein the additional base is triethylamine.

In preferred embodiments, in particular when referring to the first and the fourth aspect of the invention, A is one substituent. In preferred embodiments, A is two substituents. In preferred embodiments, B is one substituent. In preferred embodiments, B is two substituents. Preferably, when B is two substituents, B is a substituent at the carbon positions 2 and 3, more preferably B is the substituent —Cl at the carbon positions 2 and 3. In preferred embodiments, A and B are each one substituent.

In preferred embodiments, in particular when referring to the first and the fourth aspect of the invention, A is one substituent. In preferred embodiments, A is two substituents. In preferred embodiments, B is one substituent. In preferred embodiments, B is two substituents. Preferably, when B is two substituents, B is a substituent at the carbon positions 3 and 4, more preferably B is the substituent —Cl at the carbon positions 3 and 4. In preferred embodiments, A and B are each one substituent.

In the context of the invention, with respect to the structure (I), when referring to the carbon positions of A and/or B, the numbering preferably refers to the following:

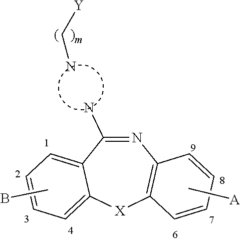

Preferably, in the Synthesis Routes A-C, the positions of the substituents A and B for each the respective reactants is selected in agreement with the positioning of the substituents A and B in the structure of (I).

In preferred embodiments of the invention, when referring to the chemical reaction steps of the invention,

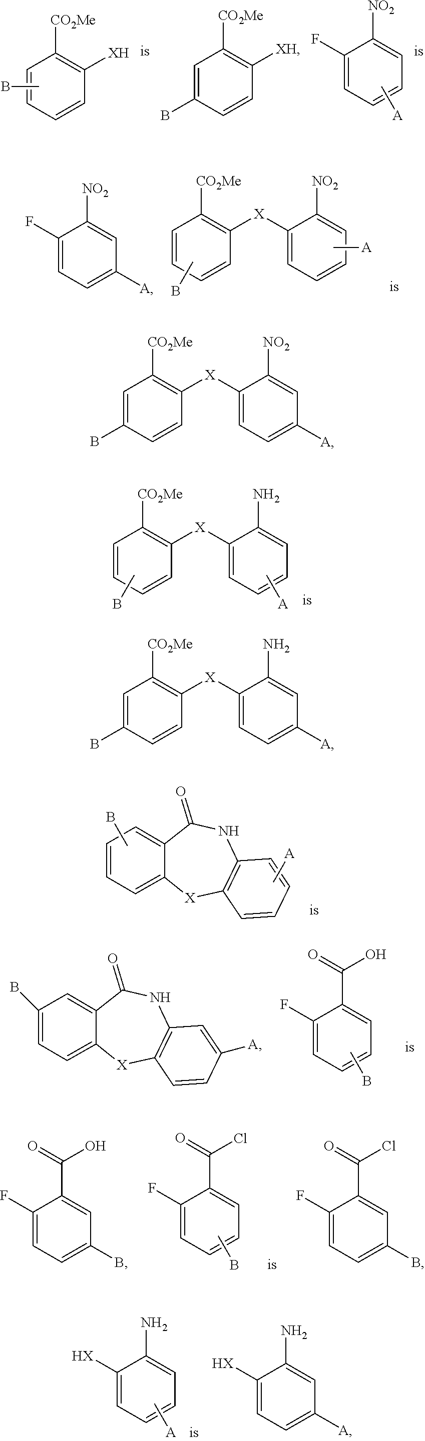

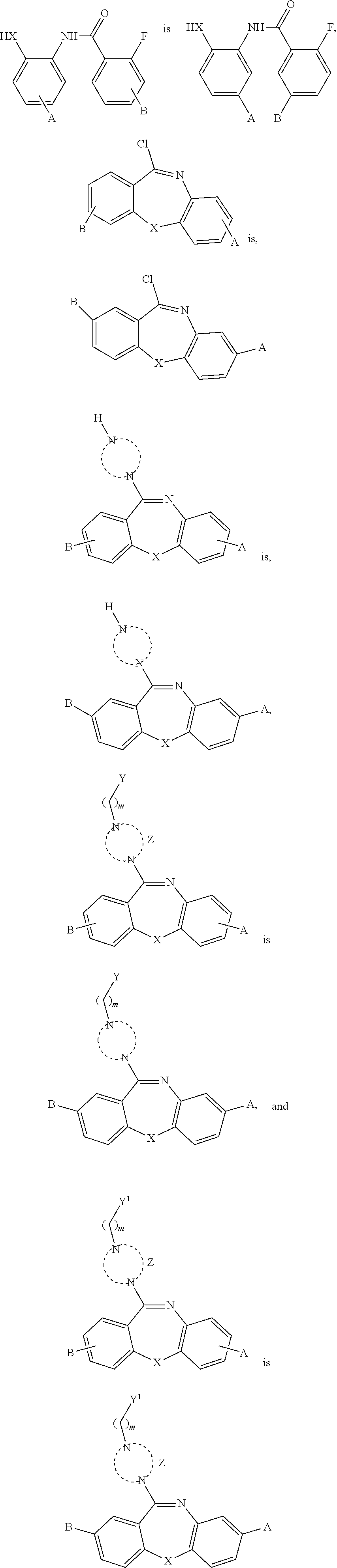

respectively.

In a further special aspect, the invention pertains to a compound, wherein the compound has the formula I:

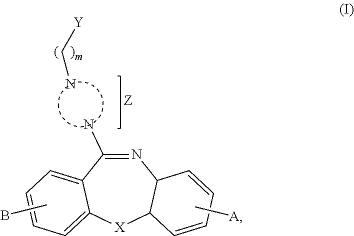

or salt, complex, diastereomer, enantiomer and/or tautomer of a compound with the formula (I), wherein
A is one or two substituents independently selected from —H, —F, —Cl, —Br, —I, —CF₃, —OCF₃, —CF₂H, and —OCF₂H;
B is one or two substituents independently selected from —F, —Cl, —Br, —I, —CF₃, —OCF₃, —CF₂H, and —OCF₂H;
X is selected from —S-and-O—;
m is 0 to 3;
Y is selected from
   —H;
   a nitrile group,
   a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O; or
   a linear —C₁₋₃alkyl, wherein a, preferably one, —(CH₂)— of the linear —C₁₋₃alkyl is optionally monosubstituted with —OH, and wherein the terminal —CH₃ of the linear —C₁₋₃alkyl is monosubstituted with —OR¹ or —SR¹, preferably —OR¹, wherein R¹ is —H, —(CH₂)—COOH or —(CH₂)—(CH₂)—OH; or
   —(CH₂)ᵢ—CX¹R², wherein
      X¹ is ═NR³ or ═O, wherein R³ is —H or —OH.
      i is 0 or 1, and
      R² is —OR⁴ or —NR⁵R⁶, wherein R⁴ is —H or —C₁₋₂alkyl; wherein R⁵ and R⁶ are independently selected from —H or -methyl;

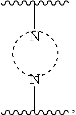

herein denoted as “Z”, is an aliphatic group comprising at least one, preferably 1 or 2, heterocycle(s) which comprise together at least two nitrogen atoms,
wherein the first of the two nitrogen atoms connects Z to

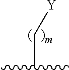

of formula I and the second of the two nitrogen atom connects Z to

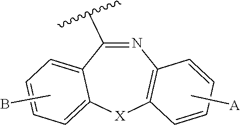

of formula I, preferably to

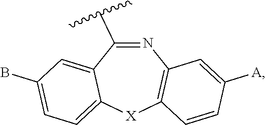

and wherein within group Z:
   the two nitrogen atoms are each a heteroatom in one of two connected 4-membered heterocycles (each heterocycle having no more than one heteroatom), wherein the two connected 4-membered heterocycles form a spirocycle, preferably wherein the 4-membered heterocycle with the first nitrogen atom is connected via a shared carbon atom to the 4-membered heterocycle with the second nitrogen atom, thus forming a spirocycle with two heteroatoms that are the two nitrogen atoms; or
   the first of the two nitrogen atoms is connected to a 4-membered heterocycle with one heteroatom, wherein the second of the two nitrogen atoms is the heteroatom in wherein the first nitrogen atom is further substituted with methyl; or
   the two nitrogen atoms are heteroatoms in a 6- to 7-membered heterocycle with two heteroatoms, wherein the heterocycle is optionally substituted, preferably monosubstituted, with methyl.

The invention furthermore pertains to the following itemized embodiments. These itemized embodiments can be combined with any of the aspects, embodiments and claims herein.

Item 1: A compound, wherein the compound has the formula:

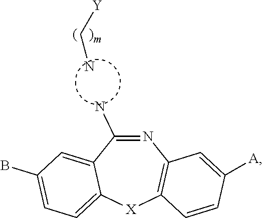

or a salt, complex, diastereomer, enantiomer and/or tautomer of thereof,
wherein
A is —H;
B is one substituent selected from —F, —Cl, —Br, —I, —CF₃, —OCF₃, —CF₂H, and —OCF₂H;
X is selected from —S-and-O—;
m is 0 to 3;
Y is selected from
   a nitrile group,
   a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O; or
   a linear —C₁₋₃alkyl, wherein a, preferably one, —(CH₂)— of the linear —C₁₋₃alkyl is optionally monosubstituted with —OH, and wherein the terminal —CH₃ of the linear-C₁₋₃alkyl is monosubstituted with —OR¹ or —SR¹, preferably —OR¹, wherein R¹ is H, —(CH₂)—COOH or —(CH₂)—(CH₂)—OH; or
   —(CH₂)ᵢ—CX¹R², wherein
      X¹ is ═NR³ or ═O, wherein R³ is —H or —OH.
      i is 0 or 1, and
      R² is —OR⁴ or —NR⁵R⁶, wherein R⁴ is —H or —C₁₋₂alkyl; wherein R⁵ and R⁶ are independently selected from —H or -methyl;

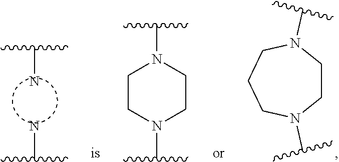

and wherein the compound is not any of,

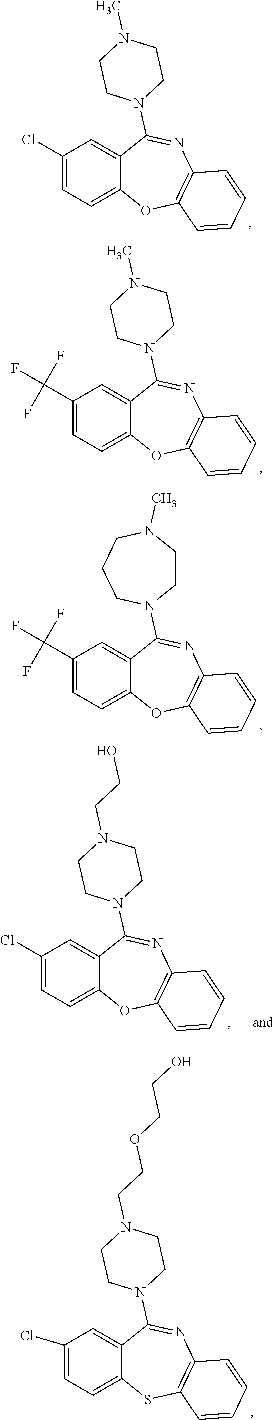

Item 2: The compound of item 1, wherein
(v) A is selected from —H.
(vi) B is selected from —Cl or —CF₃.
(vii) m is 0 to 2, more preferably m is 0 or 1, most preferably m is 1.
(viii) Y is selected from the group consisting of

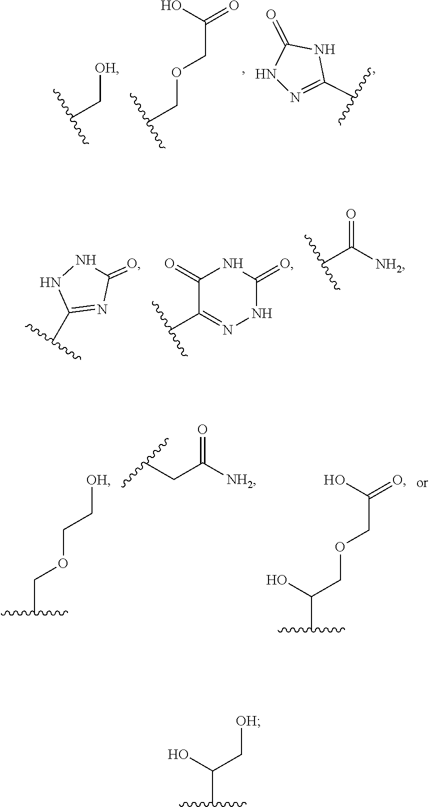

Item 3: The compound according to items 1 or 2, wherein the compound is selected from the group,

| Compound No. | Structure |
|---|---|
| 6 | 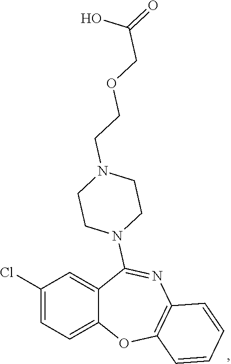 [SMILES: O=C(O)COCCN1CCN(C2=Nc3ccccc3Oc3ccc(Cl)cc32)CC1] |
| 8 | 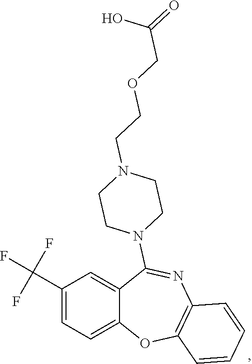 [SMILES: O=C(O)COCCN1CCN(C2=Nc3ccccc3Oc3ccc(C(F)(F)F)cc32)CC1] |
| 9 | 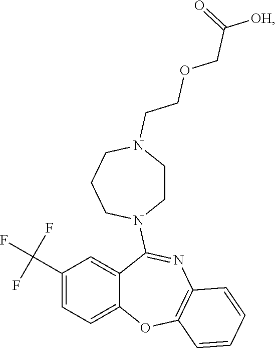 [SMILES: O=C(O)COCCN1CCCN(C2=Nc3ccccc3Oc3ccc(C(F)(F)F)cc32)CC1] |
| 10 | 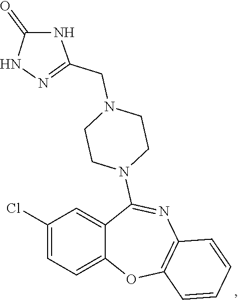 [SMILES: O=c1[nH]nc(CN2CCN(C3=Nc4ccccc4Oc4ccc(Cl)cc43)CC2)[nH]1] |
| 11 | 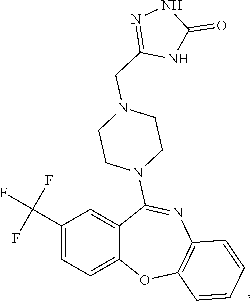 [SMILES: O=c1[nH]nc(CN2CCN(C3=Nc4ccccc4Oc4ccc(C(F)(F)F)cc43)CC2)[nH]1] |
| 12 | 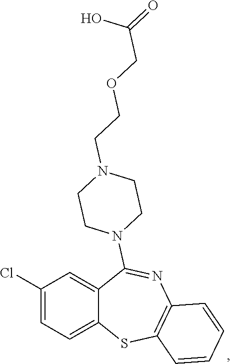 [SMILES: O=C(O)COCCN1CCN(C2=Nc3ccccc3Sc3ccc(Cl)cc32)CC1] |
| 15 | 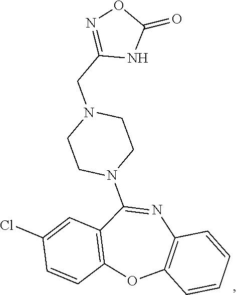 [SMILES: O=c1[nH]c(CN2CCN(C3=Nc4ccccc4Oc4ccc(Cl)cc43)CC2)no1] |
| 16 | 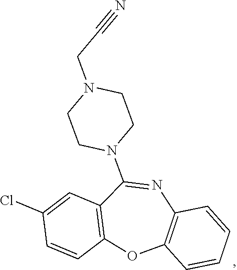 [SMILES: N#CCN1CCN(C2=Nc3ccccc3Oc3ccc(Cl)cc32)CC1] |
| 17 | 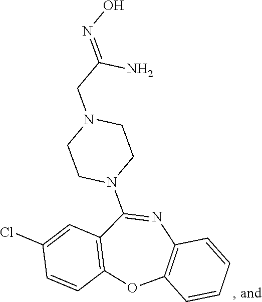 [SMILES: N/C(CN1CCN(C2=Nc3ccccc3Oc3ccc(Cl)cc32)CC1)=N\O] |
| 20 | 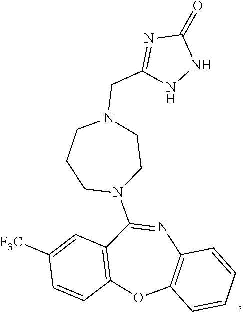 [SMILES: O=c1nc(CN2CCCN(C3=Nc4ccccc4Oc4ccc(C(F)(F)F)cc43)CC2)[nH][nH]1] |

preferably wherein compound is selected from the group consisting of compounds 6, 8, 9, 10, 11, 12, 18, 19, 20, 21, more preferably the compound is selected from the group consisting of the compounds 6, 8, 9, and 10.

Item 4: The compound of any one of claims 1 to 3, wherein the compound is

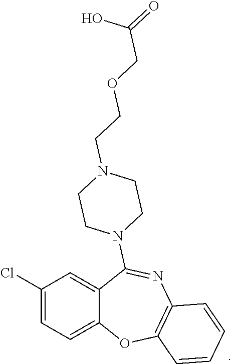 [SMILES: O=C(O)COCCN1CCN(C2=Nc3ccccc3Oc3ccc(Cl)cc32)CC1]

Item 5: The compound of any one of items 1 to 4, wherein the compound has an activity as an activator of a potassium channel in a cell, preferably of a potassium channel Slack (K_{Na}1.1), preferably wherein the compound activates the potassium channel with an EC50 of less than 100 μM, preferable of between 0.5 and 80 μM.

Item 6: The compound of any one of items 1 to 5, wherein the compound
when administered to an animal does not penetrate the blood brain barrier (BBB), or wherein the compound penetrates the BBB less than a reference compound, such as Loxapine.
does not bind the human dopamine receptor, more preferably wherein the compound binds the human dopamine receptor less compared to a reference compound, such as Loxapine.

Item 7: The compound of any one of items 1 to 6, wherein the compound is isolated and/or wherein the compound has a purity of at least 75%, optionally at least 90%, optionally at least 95%.

Item 8: A pharmaceutical composition comprising the compound of any one of items 1 to 7, or a salt, solvate, or ester thereof, and a pharmaceutically acceptable carrier or excipient.

Item 9: The pharmaceutical composition of item 8, wherein the compound, or a salt, solvate, or ester thereof, is present in an amount of 0.1% w/w to 10% w/w, optionally in an amount of 1% w/w to 10% w/w.

Item 10: A compound or composition for use in the treatment of a condition in a subject, the compound or composition comprising an effective amount of a compound with the formula:

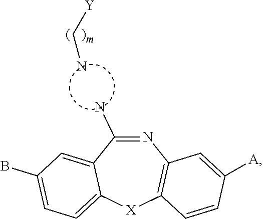

or a salt, complex, diastereomer, enantiomer and/or tautomer of thereof,
wherein
A is —H;
B is one substituent selected from —F, —Cl, —Br, —I, —CF₃, —OCF₃, —CF₂H, and —OCF₂H;
X is selected from —S-and-O—;
m is 0 to 3;
Y is selected from
   a nitrile group,
   a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O; or
   a linear —C₁₋₃alkyl, wherein a, preferably one, —(CH₂)— of the linear —C₁₋₃alkyl is optionally monosubstituted with —OH, and wherein the terminal —CH₃ of the linear-C₁₋₃alkyl is monosubstituted with —OR¹ or —SR¹, preferably —OR¹, wherein R¹ is H, —(CH₂)—COOH or —(CH₂)—(CH₂)—OH; or
   —(CH₂)ᵢ—CX¹R², wherein
      X¹ is ═NR³ or ═O, wherein R³ is —H or —OH.
      i is 0 or 1, and
      R² is —OR⁴ or —NR⁵R⁶, wherein R⁴ is —H or —C₁₋₂alkyl; wherein R⁵ and R⁶ are independently selected from —H or -methyl;

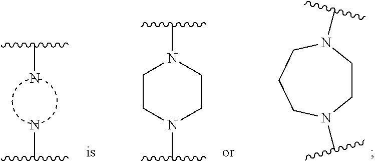

preferably the compound of any one of items 1 to 6, or a salt, solvate, or ester thereof, preferably wherein the composition is the pharmaceutical composition of items 7 or 8, wherein the condition is treatable by activating a potassium channel in a cell associated with the pathology of the condition, the treatment comprising administering the compound or composition to the subject in need thereof.

Item 11: The compound or composition for use of item 10, wherein the condition is a pruritic condition, such as an acute or chronic pruritus, optionally wherein the pruritic condition is associated with a second pathology, such as one selected from:
a dermatological disorder such as xerosis or xeroderma (dry skin), dermatitis or eczema (e.g., atopic dermatitis), psoriasis (e.g., plaque psoriasis), prurigo (e.g., prurigo nodularis), urticaria (e.g., chronic idiopathic urticaria), a connective tissue disorder (e.g., dermatomyositis), post-burn pruritus;
a kidney disorder (e.g., chronic kidney disease, chronic kidney failure or end-stage renal disease), dialysis (e.g., hemodialysis), uremic pruritus;
a hepato-biliary disorder (e.g., cholestasis, primary biliary cholangitis, primary sclerosing cholangitis, secondary sclerosing cholangitis, hepatitis, toxic liver disease, chronic liver disease or cirrhosis), cholestatic pruritus;
an endocrine disorder (e.g., hyperthyroidism or diabetes mellitus);
a metabolic disorder (e.g., iron deficiency or iron overload);
a benign or malignant neoplasm (e.g., a solid tumor, a carcinoma or a hematological neoplasm [e.g., Hodgkin's lymphoma, non-Hodgkin's lymphoma, myeloproliferative disease, or polycythemia vera]);
an infectious disease (e.g., a viral infection such as an infection with herpes simplex, herpes zoster, varicella, human immunodeficiency virus (HIV), hepatitis; a bacterial infection or a parasitosis);
a neurological disorder (e.g., a degenerative neurological disease, multiple sclerosis, a brain tumor, postherpetic neuralgia, small-fiber neuropathies, brachioradial pruritus or notalgia paresthetica), neuropathic itch, neurogenic itch;
a psychiatric disease (e.g., depression, obsessive compulsive disorder, delusional disorder, eating disorder, or anxiety);
drug-induced pruritus (e.g., by opioids, antibiotics, antimalarial agents, ACE inhibitors, angiotensin receptor antagonists, anti-arrhythmic agents, antidepressants, antidiabetic drugs, antihypertensive drugs, anticonvulsants, anti-inflammatory drugs, betablockers, bronchodilators, calcium antagonists, diuretics, hormones, immunosuppressive drugs, antilipids, neuroleptics, plasma expanders, tranquillizers, or uricostatics);
pruritus in the elderly;
pruritus in pregnancy; and/or
chronic idiopathic pruritus.

Item 12: The compound or composition for use of item 10 or 11, wherein the condition is pain or a pain related noxious sensation in the subject, preferably wherein the pain is selected from
neuropathic pain induced by traumatic nerve injury, cancer and cancer treatments (e.g., chemotherapy), neurological conditions (e.g., multiple sclerosis), neurodegenerative conditions (e.g., Parkinson's disease), trigeminal neuralgia, diabetic peripheral neuropathy, stroke, shingles, HIV, Hansen's disease (leprosy), Guillain-Barre syndrome, blood vessel disease, vascular malformations, autoimmune conditions
acute post-operative pain,
inflammatory pain, rheumatoid arthritis, osteoarthritis, or/and
nociplastic pain.

Item 13: The compound or composition for use of any one of items 10 to 12, wherein administering comprises intravenous, intraperitoneal, subcutaneous, intramuscular, intrathecal, peridural, topical, oral, gastric and/or rectal administration.

Item 14: The compound or composition for use of any one of items 10 to 13, wherein the compound is Compound 6.

Item 15: A method of synthesizing a compound according to any one of items 1 to 7, preferably, wherein the method comprises a synthesis step of synthesizing a lactam core, preferably wherein said lactam core comprises the structure:

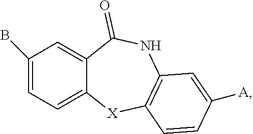

wherein A, X and B are defined as in any one of items 1 to 7.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1: Expression of Kcnt1 and critical itch receptors across sensory neuron subsets from published single-cell RNA-seq data. (A) Expression pattern in mouse DRG neurons (6-8 week old)⁴. (B) Expression pattern in human DRG neurons (24-65 year old)¹⁶. Downloaded from: https://sensoryomics.shinyapps.io/RNA-Data/. (C) Expression pattern in non-human primate DRG neurons (5-14 year old)¹⁷. Downloaded from: https://ernforsgroup.shinyapps.io/macaquedr.

FIG. 2: Establishing a modified version of the FluxOR assay. Cultured HEK293 cells stably expressing human Slack (HEK-Slack cells) were incubated with compounds of the invention in different buffers. (A) In the buffer provided with the FluxOR assay kit, Slack activation (indicated as increased F/F (Baseline) ratio) was detected after incubation with both Loxapine and vehicle (FluxOR assay buffer containing 0.03% DMSO). As Slack is activated by Na⁺, the vehicle-induced Slack activation was most likely mediated by Na⁺ present in the FluxOR assay buffer and in the vehicle. (B) In a Na⁺-free buffer (with replacement of NaCl by choline chloride), Slack activation was observed after incubation with 30 μM Loxapine and 140 mM NaCl, but not after incubation with vehicle (Na⁺ free buffer with 2 mM Ca²⁺, 2 mM Mg²⁺ and 0.03% DMSO). (C) DMSO at a concentration of 0.03-3% did not activate Slack in a Na⁺-free buffer with 2 mM Ca²⁺ and 2 mM Mg²⁺. (D) Dose-response experiments with Loxapine in a Na⁺ free buffer with 2 mM Ca²⁺, 2 mM Mg²⁺ and 0.03% DMSO yielded an EC₅₀ value of 23.45 μM in the first preliminary experiments. (E) Dose-response experiments with the Slack inhibitor compound 31 (ref¹⁸) in a Na⁺ free buffer with 2 mM Ca²⁺, 2 mM Mg²⁺ and 0.03% DMSO and pre-stimulation with 25 μM Loxapine revealed that compound 31 inhibited the F/F (Baseline) ratio in a dose-dependent manner (IC₅₀=2.1 μM), confirming that the readout depends on Slack. All further experiments with the FluxOR, which are presented in FIG. 3A, B, were performed using a Na⁺ free buffer with 2 mM Ca²⁺, 2 mM Mg²⁺ and 0.03% DMSO. All conditions were measured at least in triplicate and data are shown as mean±SD.

FIG. 3: New compounds are activators of Slack channels. (A) and (B) Dose-response experiments with new compounds in a FluxOR potassium ion channel assay. HEK293 cells stably expressing human Slack (HEK-Slack cells) were assayed for a potassium channel-mediated thallium response using FluxOR. Each compound was incubated at six concentrations. For each sample, the fluorescence value was calculated and then normalized to the maximum fluorescence value of Loxapine. For better comparability, data from the Loxapine measurements are presented in all five graphs. Each data point is the average of 3 replicates. Data represent the mean±s.d. (C) Patch-clamp recordings confirm that new compounds evoke Slack-mediated potassium currents. Whole-cell voltage recordings on HEK-Slack cells were performed at baseline and after incubation with a new compound (50 μM), Loxapine (lox; 50 μM) or vehicle (external solution containing 0.03% DMSO). Loxapine was incubated in each series of experiments as a positive control. Shown are current densities (pA/pF) of vehicle, Loxapine and new compounds relative to baseline at a voltage of +80 mV (fold increase). Corresponding Current-voltage curves from these recordings and representative outward potassium traces are presented in FIG. 4. ** P<0.01, *** P<0.001 vs vehicle (Kruskal-Wallis test with Dunn's correction); box-and-whisker plots represent maximum and minimum values, and the box shows the first, second (median) and third quartile values. (D) Correlation of relative EMAX values of new compounds obtained in the FluxOR assay (presented in (A) and (B)) with relative current densities obtained in the patch-clamp experiments (presented in (C)). Pearson correlation. (E) Time-dependent fluorescence/fluorescenceBaseline ratios of the Dose-response experiments presented in (B) in comparison with Loxapine and vehicle.

FIG. 4: New compounds evoke Slack-mediated potassium currents. Whole-cell voltage recordings on HEK-Slack cells were performed at baseline and after incubation with a new compound (50 μM), Loxapine (50 μM) or vehicle (external solution containing 0.03% DMSO). (A) Current-voltage (I-V) curves from patch-clamp experiments that are presented in FIG. 3C. Loxapine was incubated in each series of experiments as a positive control. n=8-20 cells per group. Data are shown as mean±SD. (B) Representative outward K⁺ (I_{K}) traces of Loxapine at +80 mV. (C) Current-voltage (I-V) curves from patch-clamp experiments that are presented in FIG. 3B. Loxapine was incubated in each series of experiments as a positive control. n=8-20 cells per group.

FIG. 5: In vitro and pharmacokinetic characterization of the new compounds. Blood brain barrier (BBB) permeability of the compounds of the invention was estimated using a BBB-specific parallel artificial membrane permeability assay. The predicted extent of BBB permeability is reflected by log Pₑ values, and the fraction of solute lost to the membrane in this assay is reflected by the membrane retention factor (MR). Note that a high MR value limits the predictive validity of a corresponding log Pₑ value.

FIG. 6: In vivo Pharmacokinetic profiles of new compounds. (A) Time courses of brain and plasma concentrations of the compounds 1 to 10 in mice. Animals were i.p. injected with 1 mg/kg of each compound in a cassette dosing procedure (with simultaneous administration of 3-4 compounds per animal) and plasma and brain levels were measured at different time points by LC-MS analysis. Note that the y-axes are scaled differently in all diagrams. Data are presented as mean±s.e.m. of 3 mice per group. (B) In vivo brain/plasma ratio of the new compounds in mice. Note that Compound 6 and Compound 10 have a particularly low brain/plasma ratio, suggesting a limited brain penetration. Data represent the mean. Additional pharmacokinetic parameters are presented in Table 6.

FIG. 7: In vitro pharmacology screening of Compound 6, Compound 10 and Loxapine. Compounds were tested at a concentration of 10 M in binding and enzyme and uptake assays of 44 targets (mostly human; except BZD, NMDA, MAO-A, Ca²⁺ channel, K_{V} channel and Na⁺ channel, which were from rat). Compound binding was calculated as a % inhibition of the binding of a radioactively labeled ligand (agonist or antagonist, as indicated in brackets) specific for each target. Compound enzyme inhibition effect was calculated as a % inhibition of control enzyme activity. Results showing an inhibition (or stimulation for assays run in basal conditions) higher than 50% are considered to represent significant effects of the test compounds and are presented in black. Results showing an inhibition or stimulation between 25% and 50% (indicative of weak to moderate effects) and those lower than 25% (considered mostly attributable to variability of the signal around the control level) are presented in grey. Measurements were performed in duplicate.

FIG. 8: Compound 6 and Compound 10 inhibit histamine-independent acute itch behavior and neuropathic pain behavior. (A), (B), Motor function. Compound 6, Compound 10, Loxapine or vehicle (0.9% NaCl with 10% 2-hydroxypropyl-β-cyclodextrine) were i.p. administered and (A) an accelerating rotarod test followed by (B) a vertical pole test were performed 15 min thereafter. Note that Compound 6 and 10 did not affect the time spent on the rotarod or the vertical pole, and therefore did not inhibit motor coordination, whereas Loxapine dose-dependently inhibited the motor coordination in both models. n=8 per group. Box-and-whisker plots represent maximum and minimum values, and the box shows the first, second (median) and third quartile values. Dotted lines indicate cutoff times. * p<0.05, *** p<0.001, Kruskal-Wallis test. (C)-(G), Acute itch behavior. Compound 6, Compound 10 or vehicle were i.p. administered and 15 min thereafter different pruritogens were s.c. administered into the nape of the neck and the number of scratching bouts was counted over 30 min. In each panel, the time course of scratching behavior is shown on the left and the sum of scratching bouts in 30 min is presented on the right. (C), Compound 6 and Compound 10 inhibited the scratching behavior induced by chloroquine in a dose-dependent manner. (D) Compound 6 inhibited the chloroquine-induced scratching behavior in WT mice but not in Slack^{-/-} littermates. (E) Compound 6 ameliorated the scratching behavior induced by SLIGRL, (F) but not by histamine. n=7-8 per group. Data represent the mean±s.e.m. * p<0.05, ** p<0.01, *** p<0.001; one-way-ANOVA with Dunnett's correction (C), Kruskal-Wallis test with Dunn's correction (D) or unpaired t-test (E, F). (G) Neuropathic pain behavior. In the spared nerve injury (SNI) model, neuropathic pain was induced by surgery. Twenty-eight days thereafter, mechanical hypersensitivity of the affected hindpaw (determined using a Dynamic Plantar Aesthesiometer), which is indicated by decreased paw withdrawal latency time, was detected in all mice. Then Compound 10 or vehicle were i.p. administered and the mechanical sensitivity was assessed over 3 h. Note that Compound 10 inhibited the neuropathic pain behavior. (H) Effects of Compound 6 in WT mice pretreated with a Slack inhibitor. Compound 31 (30 mg/kg) or vehicle were i.p. administered 5 min prior to Compound 6 (30 mg/kg i.p.), and chloroquine was s.c. administered 15 min thereafter. Data indicate that the antipruritic effect of Compound 6 was partly antagonized by the Slack inhibitor. n=7-8 per group. Data are shown as mean±SEM. * p<0.05, unpaired t-test.

FIG. 9: Effects of Compound 6 in models of motor function and acute itch do not show sex-related differences. Breakdown of results for Compound 6 in male and female mice from (A) FIG. 8A, (B) FIG. 8B, (C) FIGS. 8C, and (D) FIG. 8F. Statistical significance in a and b was assessed by Kruskal-Wallis test. Box-and-whisker plots represent maximum and minimum values, and the box shows the first, second (median) and third quartile values. Dotted lines indicate cutoff times. Statistical significance in c and f was assessed by one-way-ANOVA with Dunnett's correction. Data represent the mean±s.e.m.

FIG. 10: Compound 6 inhibits persistent itch behavior. (A)-(C) Efficacy of Compound 6 in the DNFB model of persistent itch. (A) Experimental diagram showing the induction of spontaneous itch behavior by topical application of 2,4-dinitrofluorobenzene (DNFB) to the nape of the neck (twice 14 days apart), i.p. drug delivery 105 min after the second DNFB application, and begin of videotaping 15 min thereafter. (B) Compound 6 significantly reduced the number of scratching bouts and (C) the number of head shakes as compared to vehicle. n=6-7 per group. (D)-(F) Efficacy of Compound 6 in the MC903 model of persistent itch. (D) Experimental diagram showing the induction of spontaneous scratching by topical application of MC903 to the nape of the neck (once daily over 7 days), i.p. drug delivery at day 8, and begin of videotaping 15 min thereafter. In comparison to vehicle, Compound 6 (E) significantly reduced the number of scratching bouts and (F) the number of head shakes. n=8 per group. Data represent the mean±s.e.m. * p<0.05, ** p<0.01, unpaired t-test.

FIG. 11: Sex-related effects of Compound 6 and correlation of behavioral outcomes in chronic itch models. (A), (B) Effects of Compound 6 in models of chronic itch do not show sex-related differences. Breakdown of results in male and female mice from (A) FIGS. 10B and 10C (DNFB model) and (B) FIGS. 10E and 10F (MC903 model). Data represent the mean±s.e.m. (C), (D), Correlation of number of head shakes with number of scratching bouts from (C) FIGS. 10B and 10C, (D) FIGS. 10E and 10F and (E) combination thereof. Statistical significance was assessed by a Pearson correlation.

FIG. 12: Potential side effects of Compound 6. Pulse oximetry on non-anesthetized mice using a MouseOX Plus device revealed that i.p. delivery of Compound 6 or vehicle did not alter (A) the heart rate or (B) the breath rate, whereas morphine significantly lowered both parameters (n=6 per group). * p<0.05, ** p<0.01, *** p<0.001 vs vehicle, two-way MC ANOVA and Dunnett test.

FIG. 13: Compound 6 reduces neuronal excitability of itch-sensitive sensory neurons. Cultured DRG neurons from mice were incubated overnight with an inflammatory soup followed by whole cell current-clamp recordings on IB4-binding neurons. (A) Recordings from a DRG neuron showing action potential (AP) firing in response to current injections (200-950 pA at 150 pA intervals, 1000-ms duration) at baseline, in the presence of Compound 6 (50 M), and after wash-out. (B) Group data show that Compound 6 significantly blocked AP firing compared to baseline; n=6 neurons. (C) Recordings from a DRG neuron showing a single AP evoked by injections of small currents (0-220 pA at 20 pA intervals, 10-ms duration) at baseline and in the presence of Compound 6 (50 μM). (D), (E) Group data indicate that Compound 6 (D) significantly increased the rheobase (amount of current required to generate an AP; and (E) reduced the AP amplitude; n=7 neurons. Data represent the mean±s.e.m. * p<0.05, ** p<0.01, *** p<0.001, paired t-test.

FIG. 14: Antinociceptive effects of Compound 6 in the CFA-induced inflammatory pain model in mice. Mechanical sensitivity of a hindpaw was determined using a dynamic plantar aesthesiometer. Twenty-four hours after CFA injection into a hindpaw mice were intraperitoneally treated with Compound 6 (10 mg/kg) or vehicle (10% cyclodextrine in 0.9% NaCl). Note that paw withdrawal latencies were significantly increased 0.5 h and 1 h after administration of Compound 6 (n=11-12 mice per group). Results are presented as the mean±SEM. Data were analyzed by two-way repeated measures ANOVA with Šídak's post hoc test. * P<0.05, ** P<0.01.

FIG. 15: Topically administered Slack activators inhibit histamine-independent itch behavior. Compound 6, Compound 21, or vehicle were topically applied on the nape of the neck. After 30 min, chloroquine was s.c. administered into the nape of the neck and the number of scratching bouts was counted over 30 min. The time course of scratching behavior is shown on the left and the sum of scratching bouts in 30 min is presented on the right. Note that both Compound 6 and Compound 21 significantly inhibited the scratching behavior induced by chloroquine. Data represent the mean±SEM (n=6). * P<0.05, ** P<0.01.

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### Example 1: Synthesis of Slack-Activating Compounds

A series of new Slack-activating compounds were developed based on the antipsychotic drug Loxapine. Synthesis of these new compounds was performed by linear synthesis Routes A-C under argon atmosphere as described below.

Synthesis Route A: The Loxapine scaffold contains a tricyclic core and a piperazine ring substituted by an alkyl residue. Starting from methyl esters of salicylic acid and 2-fluoronitrobenzene derivatives, diarylethers were obtained by nucleophilic aromatic substitution reaction. Subsequent reduction of the nitro group by tin (II) chloride enabled intramolecular amide bond formation.

### Synthesis Route A

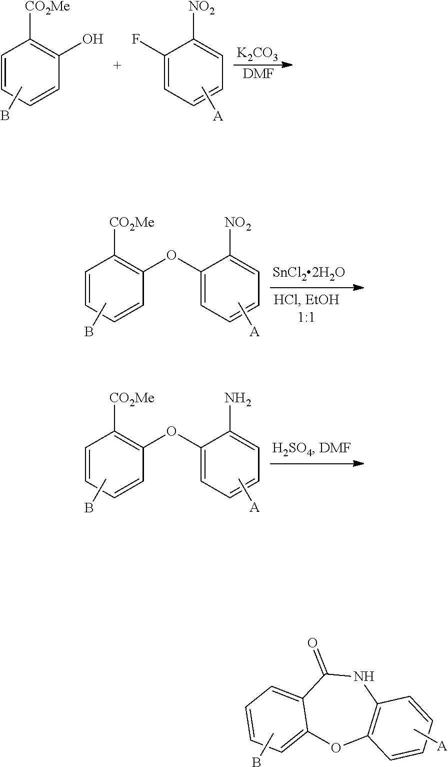

Synthesis Route B: Alternatively, amide bond was first formed between 2-fluorobenzoic acids and 2-aminophenol derivatives, followed by intramolecular nucleophilic aromatic substitution to afford the same lactam core.

### Synthesis Route B

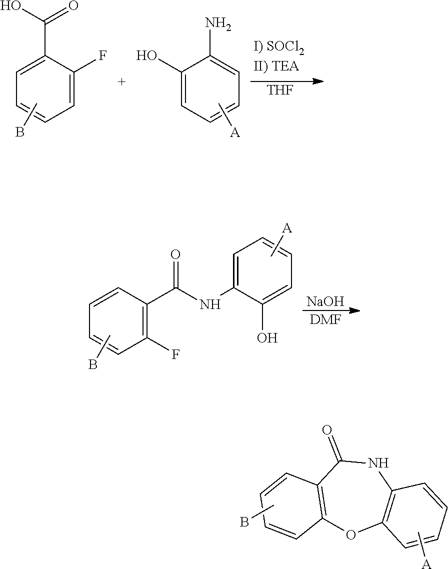

Synthesis Route C: The lactam was then converted to an imidoyl chloride by POCl₃. In the last step, substituted piperazines displaced the chloride to afford the desired Loxapine derivatives.

### Synthesis Route C

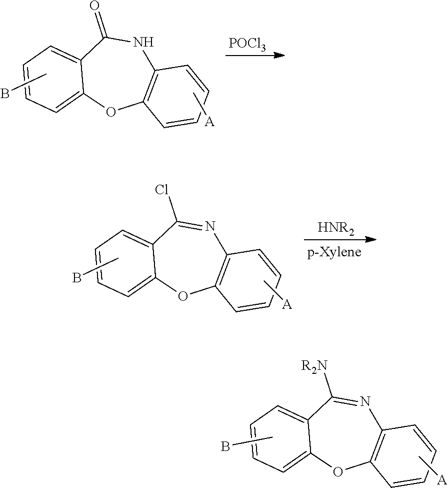

Synthesis Route A—General Synthesis of Lactam Starting from Methyl Esters of Salicylic Acid and 2-Fluoronitrobenzene Derivatives.

GP1—Formation of the diarylether: Potassium carbonate (1.5 equiv) was added to a solution of the corresponding methyl salicylate (1.5 equiv) and the corresponding 2-fluoronitrobenzene (1.0 equiv) in DMF (1.5 M regarding 2-fluoronitrobenzene). The resulting solution was heated in an oil bath to 120° C. overnight. Solvents were evaporated under reduced pressure and the residue taken into water and extracted three times with ethyl acetate. The combined organic phases were dried over magnesium sulfate, filtered, and evaporated. The resulting crude product was purified by flash chromatography.

GP2—Reduction of the nitro group: A solution of SnCl₂·2H₂O (4.0 equiv) in conc. HCl (3 M) was added to a solution of the corresponding methyl 2-(2-nitrophenoxy)benzoate derivative (1.0 equiv) in a mixture of ethanol/conc. HCl 1:1 (0.5 M). The resulting solution was stirred at rt overnight. After that time, the temperature was set to 0° C. and the pH of the reaction solution made slightly basic by addition of sodium carbonate. The resulting solution was then extracted three times with ethyl acetate. The combined organic phases were dried over magnesium sulfate, filtered, and evaporated. The resulting crude product was purified by flash chromatography.

### GP3—Formation of the Lactam Ring Via Intramolecular Condensation:

GP3I: A solution of the corresponding methyl 2-(2-aminophenoxy)benzoate derivative (1.0 equiv) in DMF (0.2 M) was treated with concentrated sulfuric acid (1.3 equiv) and heated in an oil bath at 120° C. overnight. After that time, the reaction was cooled to 0° C. and a few milliliters of water were added. The precipitated product was then filtered off and dried under vacuum to obtain the crude product, which was used in the next step without further purification.

Synthesis Route B—General Synthesis of Lactam Starting from 2-Fluorobenzoic Acids and 2-Aminophenol Derivatives.

GP4—Formation of the amide: To a solution of the corresponding 2-fluorobenzoic acid (1.0 equiv) in THF (1 M) freshly distilled thionyl chloride (2.0 equiv) was added and heated to reflux in an oil bath for 2 h. Thereafter, excess thionyl chloride and THF were removed under reduced pressure and the residue obtained was taken up in THF (2.5 M) again. This solution was added dropwise to a solution of the corresponding 2-aminophenol derivative (1.0 eq) and triethylamine (2 equiv) in THF (2.5 M referred to 2-aminophenol derivative) at 0° C. and the reaction mixture was stirred overnight at rt. After that time, the reaction mixture was concentrated under reduced pressure and the residue taken up in ethyl acetate. The organic phase was first washed with an aq HCl solution (2 M), water and saturated aq NaCl solution, then dried over magnesium sulfate, filtered, and evaporated. The resulting crude product was purified by flash chromatography.

GP5—Formation of the lactam ring via intramolecular nucleophilic aromatic substitution: A solution of the corresponding 2-fluoro-N-(2-hydroxyphenyl)benzamide derivative (1.0 equiv) in DMF (0.25 M) was treated with freshly powdered NaOH (1.0 equiv) and heated in an oil bath at 150° C. for 5 h. After that time, the reaction was cooled to 0° C. and a few milliliters of water were added. The precipitated product was then filtered off and dried under vacuum to obtain the crude product, which was used in the next step without further purification.

Synthesis Route C—General Synthesis of Loxapine Derivatives Starting from Lactam

GP6—Reaction with phosphorus oxychloride: The corresponding lactam (1.0 equiv) was dissolved in freshly distilled phosphorus oxychloride (0.5 M) and N,N-dimethylaniline (0.6 equiv) was added. The resulting mixture was heated to reflux in an oil bath for 5 h. Thereafter, excess phosphorus oxychloride was removed under reduced pressure and the residue obtained was taken up in toluene and washed once with cold water. The organic phase was dried over magnesium sulfate, filtered, and evaporated to give the crude product, which was immediately used in the next step without further purification.

GP7—Formation of Loxapine derivatives: The corresponding amine (2.0 eq) was added to a solution of the appropriate imidoyl chloride (1.0 equiv) in p-xylene (0.15 M regarding imidoyl chloride). The resulting mixture was heated in an oil bath at 140° C. for 5 h. Solvents were evaporated, and the crude substance purified by preparative HPLC or flash chromatography. If unsubstituted piperazine or homopiperazine was employed, then alkylation of these derivatives was obtained by GP8.

GP8—Alkylation of Loxapine derivatives: A mixture of Loxapine derivative (1 equiv), corresponding alkyl chloride (2 equiv.) and triethylamine (10 equiv.) in acetonitrile (0.15 M) was heated to reflux in an oil bath for 16 h. After completion, the reaction mixture was evaporated, and the residue purified by preparative HPLC or flash chromatography.

### Example 2: Characterization of Synthesized Compounds

Using the Synthesis Routes as described above, the compounds were synthesized. For characterization of these compounds, NMR spectra were recorded with Bruker Avance DPX250, Bruker Avance 300, Bruker Avance 400, or Bruker Avance 500 all from Bruker (Karlsruhe, Germany) operating at ambient temperature. Proton spectra were recorded in CDCl₃ or DMSO-d₆ and ¹H NMR chemical shifts were referenced to the residual signals of CHCl₃ (at δ=7.26 ppm) and DMSO-d₅ (at δ=2.50 ppm). ¹³C NMR chemical shifts were referenced against the central line of the solvent signal (for CDCl₃ at δ=77.16 ppm and for DMSO-d₆ at δ=39.52 ppm). Chemical shifts are given on δ scale (ppm). Coupling constants (J) are given in Hz. Multiplicities are indicated as follows: br (broad signal), s (singlet), d (doublet), t (triplet), q (quartet), quint or m (multiplet). ESI-MS were measured with LCMS-2020 from Shimadzu and HRMS with MALDI Orbitrap XL from Thermo Scientific. TLC was carried out on silica gel plates from Marcherey-Nagel (ALUGRAM®) and visualized with an UV lamp (254 nm and/or 366 nm). Purification of products was performed by flash chromatography using puriFlash XS420 and Silica HP 30 μm columns as stationary phase all from Interchim (Montluçon, France). Analytical and semipreparative HPLC was conducted by Shimadzu prominence with a SPD20A UV/Vis detector both from Shimadzu (Duisburg, Germany). Stationary phases were Luna 10 μm 100 Å, C18 (2) (250×4.6 mm) and Luna 10 μm 100 Å, C18 (2) (250×21.20 mm) from Phenomenex (Aschaffenburg, Germany) and eluent was a mixture of ACN and aq formic acid solution (0.1%). Flow rate was set to 1 mL/min and 21 mL/min. All compounds tested display a purity of >95% (254 nm).

### 11-(4-Methylpiperazin-1-yl)-2-(trifluoromethyl)dibenzo[b,f][1,4]oxazepine (Compound 2)

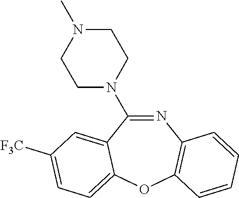 [SMILES: CN1CCN(C2=Nc3ccccc3Oc3ccc(C(F)(F)F)cc32)CC1]

Synthesized according to GP7 from 11-chloro-2-(trifluoromethyl)dibenzo[b,f][1,4]oxazepine (63 mg, 0.210 mmol) and 1-methylpiperazine (46.6 μL, 0.420 mmol). Purification of the crude product by preparative HPLC yielded the title compound as a brown oil and formate salt (22 mg, 26%). ¹H NMR (300 MHz, CDCl₃) δ 8.35 (s, 1H), 7.95 (br s, 1H), 7.70 (ddd, J=8.5, 2.3, 0.5 Hz, 1H), 7.62 (d, J=2.3 Hz, 1H), 7.36 (d, J=8.5 Hz, 1H), 7.18-7.07 (m, 3H), 7.01 (td, J=7.4, 2.0 Hz, 1H), 3.62 (br s, 4H), 2.69 (br s, 4H), 2.44 (s, 3H); 13C[1H] NMR (75 MHz, CDCl₃) δ 166.6, 163.2, 158.7, 151.4, 139.9, 129.7, 127.5, 127.2, 127.0, 126.0, 124.9, 124.1, 123.5, 122.1, 120.3, 54.0, 46.7, 45.3; HPLC-purity (254 nm): 96%; MALDI-HRMS: m/z calculated for C₁₉H₁₉F₃N₃O [M+H]+: 362.1474, found: 362.1488.

### 11-(4-Methyl-1,4-diazepan-1-yl)-2-(trifluoromethyl)dibenzo[b,f][1,4]oxazepine (Compound 3)

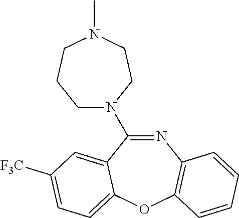 [SMILES: CN1CCCN(C2=Nc3ccccc3Oc3ccc(C(F)(F)F)cc32)CC1]

Synthesized according to GP7 from 11-chloro-2-(trifluoromethyl)dibenzo[b,f][1,4]oxazepine (63 mg, 0.212 mmol) and 1-methylhomopiperazine (49 mg, 0.423 mmol). Purification of the crude product by preparative HPLC yielded the title compound as a brown oil and formate salt (73 mg, 92%). ¹H NMR (300 MHz, CDCl₃) δ 9.97 (br s, 1H), 8.51 (s, 1H), 7.68 (dd, J=8.5, 1.9 Hz, 1H), 7.58 (d, J=2.1 Hz, 1H), 7.35 (d, J=8.5 Hz, 1H), 7.12-7.06 (m, 3H), 6.98-6.95 (m, 1H), 4.06-3.45 (m, 4H), 3.08-2.84 (m, 4H), 2.56 (s, 3H), 2.29 (br s, 1H), 2.01 (br s, 1H); 13C[1H] NMR (75 MHz, CDCl₃) δ 168.2, 162.8, 158.3, 151.2, 140.4, 129.4, 127.4, 127.0, 126.9, 126.2, 124.2, 124.0, 123.6, 122.2, 120.2, 56.9, 56.3, 49.1, 46.9, 45.4, 26.3; HPLC-purity (254 nm): 97%; MALDI-HRMS: m/z calculated for C₂₀H₂₁F₃N₃O [M+H]+: 376.1631, found: 376.1639.

### 8-Fluoro-11-(4-methylpiperazin-1-yl)-2-(trifluoromethyl)dibenzo[b,f][1,4]oxazepine (Compound 4)

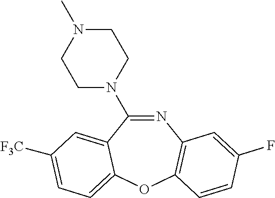 [SMILES: CN1CCN(C2=Nc3cc(F)ccc3Oc3ccc(C(F)(F)F)cc32)CC1]

Synthesized according to GP7 from 11-chloro-8-fluoro-2-(trifluoromethyl)dibenzo[b,f][1,4]oxazepine (50 mg, 0.158 mmol) and 1-methylpiperazine (32 mg, 0.317 mmol). Purification of the crude product by preparative HPLC yielded the title compound as a yellow solid (25 mg, 42%). ¹H NMR (300 MHz, CDCl₃) δ 7.68 (dd, J=8.5, 1.9 Hz, 1H), 7.58 (d, J=2.1 Hz, 1H), 7.35 (d, J=8.5 Hz, 1H), 7.12-7.06 (m, 3H), 6.98-6.95 (m, 1H), 4.06-3.45 (m, 4H), 3.08-2.84 (m, 4H), 2.56 (s, 3H), 2.29 (br s, 1H), 2.01 (br s, 1H); 13C[1H] NMR (75 MHz, CDCl₃) δ 163.2, 161.5, 159.6, 159.4, 147.7, 141.6, 128.4, 127.7, 123.7, 124.1, 122.2, 120.1, 113.4, 110.9, 54.7, 47.3, 46.04; HPLC-purity (254 nm): >99%; MALDI-HRMS: m/z calculated for C₁₉H₁₈F₄N₃O [M+H]+: 380.1381, found: 380.1377.

### 2-(4-(2-Chlorodibenzo[b,f][1,4]oxazepin-11-yl) piperazin-1-yl) ethan-1-ol (Compound 5)

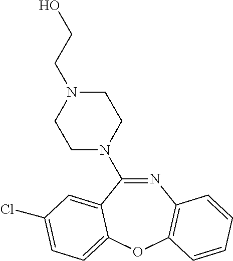 [SMILES: OCCN1CCN(C2=Nc3ccccc3Oc3ccc(Cl)cc32)CC1]

Synthesized according to GP7 from 2, 11-dichlorodibenzo[b,f][1,4]oxazepane (198 mg, 0.750 mmol) and hydroxyethylpiperazine (195 mg, 1.50 mmol). Purification of the crude product by preparative HPLC yielded the title compound as a brown oil and formate salt (216 mg, 72%). ¹H NMR (300 MHz, CDCl₃) δ 8.40 (s, 1H), 7.41 (dd, J=8.6, 2.6 Hz, 1H), 7.31 (d, J=2.6 Hz, 1H), 7.19 (d, J=8.6 Hz, 1H), 7.16-7.07 (m, 3H), 7.05-6.99 (m, 1H), 6.51 (br s, 2H), 3.85-3.81 (m, 2H), 3.74 (br s, 4H), 2.98 (br s, 4H), 2.89-2.85 (m, 2H); 13C[1H] NMR (75 MHz, CDCl₃) δ 166.8, 159.3, 158.3, 151.7, 139.6, 133.0, 130.5, 128.8, 127.1, 125.9, 125.1, 124.5, 122.9, 120.2, 59.7, 56.9, 52.2, 45.8; HPLC-purity (254 nm): 99%; MALDI-HRMS: m/z calculated for C₁₉H₂₁ClN₃O₂ [M+H]+: 358.1317, found: 358.1324.

### 2-(2-(4-(2-Chlorodibenzo[b,f][1,4]oxazepin-11-yl) piperazin-1-yl) ethoxy) acetic acid (Compound 6)

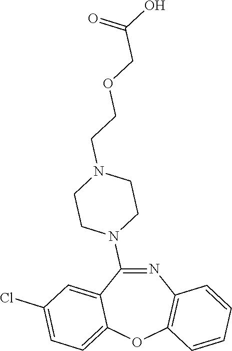 [SMILES: O=C(O)COCCN1CCN(C2=Nc3ccccc3Oc3ccc(Cl)cc32)CC1]

Synthesized according to GP8 from commercially purchased amoxapine (75 mg, 0.239 mmol), 2-(2-chloroethoxy) acetic acid (66 mg, 0.478 mmol) and triethylamine (0.34 mL, 2.39 mmol). Purification of the crude product by preparative HPLC yielded the title compound as a brown oil and formate salt (47 mg, 42%). ¹H NMR (300 MHz, CDCl₃) δ 8.24 (br s, 1H), 7.41 (dd, J=8.7, 2.4 Hz, 1H), 7.34 (d, J=2.4 Hz, 1H), 7.18 (d, J=8.7 Hz, 1H), 7.15-6.98 (m, 4H), 5.91 (br s, 2H), 4.23 (br s, 2H), 4.44-3.94 (m, 6H), 3.87-3.72 (m, 6H); 13C[1H] NMR (75 MHz, CDCl₃) δ 168.8, 164.7, 159.3, 159.0, 151.6, 139.1, 133.4, 130.8, 128.7, 127.2, 126.0, 125.7, 123.9, 123.0, 120.3, 61.4, 59.5, 55.8, 41.4; HPLC-purity (254 nm): 99%; MALDI-HRMS: m/z calculated for C21H₂₃ClN₃O₄ [M+H]+: 416.1372, found: 416.1375.

### 2-(2-(4-(2-Chlorodibenzo[b,f][1,4]thiazepin-11-yl) piperazin-1-yl) ethoxy) ethan-1-ol (Compound 7)

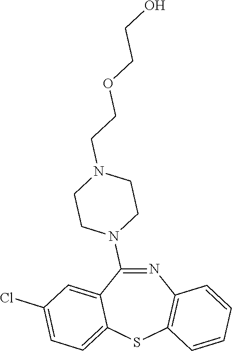 [SMILES: OCCOCCN1CCN(C2=Nc3ccccc3Sc3ccc(Cl)cc32)CC1]

Synthesized according to GP7 from 2, 11-dichlorodibenzo[b,f][1,4]thiazepine (119 mg, 0.425 mmol) and 1-[2-(2-hydroxyethoxy)ethyl]piperazine (147 μL, 0.850 mmol). Purification of the crude product by preparative HPLC yielded the title compound as a brown oil and formate salt (156 mg, 79%). ¹H NMR (300 MHz, CDCl₃) δ 8.37 (s, 1H), 7.46-7.41 (m, 1H), 7.38 (dd, J=7.7, 1.5 Hz, 1H), 7.33-7.27 (m, 2H), 7.29 (br s, 1H), 7.23-7.17 (m, 1H), 7.06 (dd, J=8.0, 1.5 Hz, 1H), 6.93 (td, J=7.3, 1.5 Hz, 1H), 3.88 (br s, 2H), 3.75 (t, J=5.1 Hz, 2H), 3.70 (t, J=4.8 Hz, 2H), 3.58 (t, J=4.8 Hz, 2H), 3.55 (br s, 2H), 3.08-3.00 (m, 2H), 2.90 (t, J=5.3 Hz, 2H), 2.90-2.82 (br s, 2H); 13C[1H] NMR (75 MHz, CDCl₃) δ 166.7, 158.9, 148.2, 138.1, 134.9, 134.8, 133.4, 132.9, 131.2, 129.4, 128.6, 127.3, 125.3, 123.6, 72.6, 66.1, 61.4, 57.2, 52.0, 45.1; HPLC-purity (254 nm): 95%; MALDI-HRMS: m/z calculated for C₂₁H₂₅ClN₃O₂S [M+H]+: 418.1351, found: 418.1350.

### 2-(2-(4-(2-(Trifluoromethyl)dibenzo[b,f][1,4]oxazepin-11-yl) piperazin-1-yl) ethoxy) acetic acid (Compound 8)

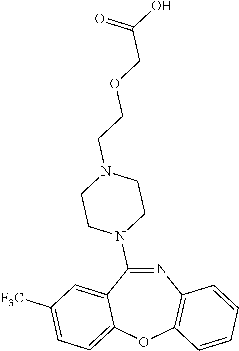 [SMILES: O=C(O)COCCN1CCN(C2=Nc3ccccc3Oc3ccc(C(F)(F)F)cc32)CC1]

Synthesized according to GP8 from 11-(piperazin-1-yl)-2-(trifluoromethyl)dibenzo[b,f][1,4]oxazepine (75 mg, 0.102 mmol), 2-(2-chloroethoxy) acetic acid (28 mg, 0.205 mmol) and triethylamine (0.14 mL, 1.02 mmol). Purification of the crude product by flash chromatography (DCM/MeOH 95:5 to 9:1) yielded the title compound as a colorless solid (28 mg, 61%). ¹H NMR (400 MHz, CDCl₃) δ 7.66 (dd, J=8.5, 2.0 Hz, 1H), 7.56-7.56 (m, 1H), 7.31 (d, J=8.5 Hz, 1H), 7.12-7.03 (m, 3H), 6.97 (td, J=7.6, 1.8 Hz, 1H), 4.21 (s, 2H), 3.73-3.43 (m, 12H); 13C[1H] NMR (101 MHz, CDCl₃) δ 168.7, 163.4, 158.8, 151.5, 139.7, 128.5, 127.8, 127.4, 126.3, 125.4, 124.1, 123.6, 122.5, 120.5, 73.8, 69.7, 61.8, 44.3, 41.7; HPLC-purity (254 nm): 98%; MALDI-HRMS: m/z calculated for C₂₂H₂₃F₃N₃O₄ [M+H]+: 450.1635, found: 450.1637.

### 2-(2-(4-(2-(Trifluoromethyl)dibenzo[b,f][1,4]oxazepin-11-yl)-1,4-diazepan-1-yl) ethoxy) acetic acid (Compound 9)

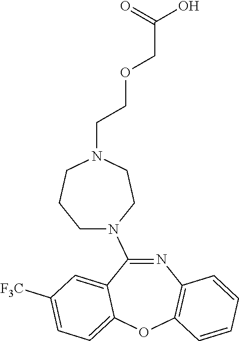 [SMILES: O=C(O)COCCN1CCCN(C2=Nc3ccccc3Oc3ccc(C(F)(F)F)cc32)CC1]

Synthesized according to GP8 from 11-(4-methyl-1,4-diazepan-1-yl)-2-(trifluoromethyl)dibenzo[b,f][1,4]oxazepine (40 mg, 0.111 mmol), 2-(2-chloroethoxy) acetic acid (31 mg, 0.221 mmol) and triethylamine (0.16 mL, 1.11 mmol). Purification of the crude product by flash chromatography (DCM/MeOH 95:5 to 9:1) yielded the title compound as a colorless solid (20 mg, 39%). ¹H NMR (400 MHz, CDCl₃) δ 7.95-7.91 (m, 1H), 7.80-7.77 (m, 1H), 7.59 (t, J=8.8 Hz, 1H), 7.22-7.18 (m, 1H), 7.11-6.93 (m, 3H), 4.63 (d, J=4.8 Hz, 1H), 4.18-4.09 (m, 3H), 3.77-3.36 (m, 10H), 2.08-1.46 (m, 2H); 13C[1H] NMR (101 MHz, CDCl₃) δ 168.6, 162.4, 157.6, 150.7, 140.3, 129.9, 126.6, 126.3, 126.0, 126.0, 123.7, 123.6, 123.7, 122.4, 120.2, 72.5, 69.1, 68.9, 68.2, 67.6, 63.2, 60.0, 44.0; HPLC-purity (254 nm): 95%; MALDI-HRMS: m/z calculated for C₂₃H₂₅F₃N₃O₄ [M+H]+: 464.1792, found: 464.1788.

### 5-((4-(2-Chlorodibenzo[b,f][1,4]oxazepin-11-yl) piperazin-1-yl)methyl)-1,2-dihydro-3H-1,2,4-triazol-3-one (Compound 10)

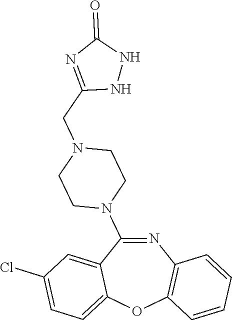 [SMILES: O=c1nc(CN2CCN(C3=Nc4ccccc4Oc4ccc(Cl)cc43)CC2)[nH][nH]1]

Synthesized according to GP8 from commercially purchased amoxapine (108 mg, 0.334 mmol), 3-(chloromethyl)-1H-1,2,4-triazol-5 (4H)-one (66 mg, 0.478 mmol) and triethylamine (0.47 mL, 3.34 mmol). Purification of the crude product by flash chromatography (EtOAc/MeOH 9:1) yielded the title compound as a colorless solid (136 mg, 99%). ¹H NMR (400 MHz, DMSO-d₆) δ 11.36 (br s, 1H), 11.26 (br s, 1H), 7.61 (dd, J=8.7, 2.6 Hz, 1H), 7.42-7.39 (m, 2H), 7.18 (dd, J=7.9, 1.2 Hz, 1H), 7.11-7.04 (m, 3H), 3.47 (br s, 4H), 3.36 (br s, 2H), 2.54 (br s, 4H); 13C[1H] NMR (101 MHz, CDCl₃) δ 158.7, 158.0, 156.2, 151.2, 144.3, 139.9, 133.0, 129.5, 128.7, 126.5, 125.8, 124.4, 124.2, 123.1, 120.2, 53.2, 52.0, 48.6; HPLC-purity (254 nm): 99%; MALDI-HRMS: m/z calculated for C₂₀H₂₀ClN₆O₂ [M+H]+: 411.1331, found: 411.1327.

### 5-((4-(2-(Trifluoromethyl)dibenzo[b,f][1,4]oxazepin-11-yl) piperazin-1-yl)methyl)-1,2-dihydro-3H-1,2,4-triazol-3-one (Compound 11)

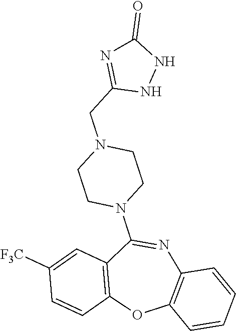 [SMILES: O=c1nc(CN2CCN(C3=Nc4ccccc4Oc4ccc(C(F)(F)F)cc43)CC2)[nH][nH]1]

Synthesized according to GP8 from Compound 13 (20.0 mg, 0.0576 mmol), 3-chloromethyl)-1H-1,2,4-triazol-5 (4H)-one (15.9 mg, 0.115 mmol), and triethylamine (80.3 μL, 0.576 mmol). Purification of the crude product by flash chromatography (DCM/MeOH 95:5 to 8:2) yielded the title compound (24.0 mg, 94%). ¹H NMR (500 MHz, DMSO-d₆) δ 11.37 (s, 1H), 11.26 (s, 1H), 7.95 (dd, J=8.7, 2.1 Hz, 1H), 7.70 (d, J=2.2 Hz, 1H), 7.60 (d, J=8.5 Hz, 1H), 7.23 (dd, J=7.9, 1.3 Hz, 1H), 7.13-7.06 (m, 2H), 7.04-7.00 (m, 1H), 3.46 (br s, 4H), 3.35 (s, 2H), 2.53 (br s, 4H). ¹³C{1H} (126 MHz, DMSO-d₆) δ 162.6, 158.1, 156.2, 150.8, 144.3, 139.9, 130.3, 126.8, 126.6, 126.2, 126.1, 123.6, 124.4, 123.6, 122.6, 120.4, 53.3, 52.0, 46.7; HPLC-purity (254 nm): 99%; MALDI-HRMS: m/z calculated for C₂₁H₂₀F₃N₆O₂ [M+H]⁺: 445.1594, found: 445.1592.

### 2-(2-(4-(2-Chlorodibenzo[b,f][1,4]thiazepin-11-yl) piperazin-1-yl) ethoxy) acetic acid (Compound 12)

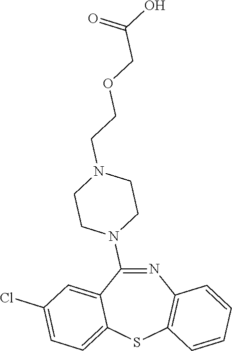 [SMILES: O=C(O)COCCN1CCN(C2=Nc3ccccc3Sc3ccc(Cl)cc32)CC1]

Synthesized according to GP8 from

2-chloro-11-(piperazin-1-yl)dibenzo[b,f][1,4]thiazepine (45 mg, 0.136 mmol), 2-(2-chloroethoxy) acetic acid (37.7 mg, 0.272 mmol), and triethylamine (191 μL, 1.36 mmol). Purification of the crude product by preparative HPLC yielded the title compound (19.6 mg, 33%). ¹H NMR (300 MHz, CDCl₃) δ 7.46 (d, J=8.3 Hz, 1H), 7.39 (dd, J=7.7, 1.3 Hz, 1H), 7.35-7.31 (m, 2H), 7.24-7.18 (m, 1H), 7.07 (dd, J=7.9, 1.2 Hz, 1H), 6.94 (td, J=7.6, 1.4 Hz, 1H), 4.25 (s, 2H), 3.84-3.36 (m, 12H). ¹³C{1H} NMR (75 MHz, CDCl₃) δ 168.5, 159.2, 148.1, 138.3, 135.1, 134.8, 133.5, 132.2, 131.3, 129.4, 128.5, 127.4, 125.3, 123.7, 73.6, 70.0, 61.7, 44.1, 41.5; HPLC-purity (254 nm): 99%; MALDI-HRMS: m/z calculated for C₂₁H₂₃ClN₃O₃S [M+H]⁺: 432.1143, found: 432.1148.

### 11-(Piperazin-1-yl)-2-(trifluoromethyl)dibenzo[b,f][1,4]oxazepane (Compound 13)

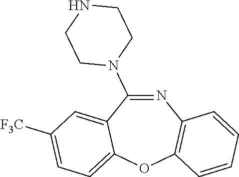 [SMILES: FC(F)(F)c1ccc2c(c1)C(N1CCNCC1)=Nc1ccccc1O2]

Synthesized according to GP7 from 11-chloro-2-(trifluoromethyl)dibenzo[b,f][1,4]oxazepine (267 mg, 0.897 mmol) and piperazine (155 mg, 1.79 mmol) in. Purification of the crude product by flash chromatography (DCM/MeOH 95:5 to 9:1) yielded the title compound as a yellow solid (240 mg, 77%). ¹H NMR (400 MHz, CDCl₃) δ 8.77 (s, 2H), 8.47 (br s, 1H), 7.73 (d, J=8.0 Hz, 1H), 7.64-7.60 (m, 1H), 7.37 (d, J=8.0 Hz, 1H), 7.18-7.03 (m, 4H), 3.77-3.23 (m, 8H).

### 11-(1,4-Diazepan-1-yl)-2-(trifluoromethyl)dibenzo[b,f][1,4]oxazepine (Compound 14)

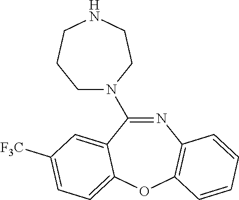 [SMILES: FC(F)(F)c1ccc2c(c1)C(N1CCCNCC1)=Nc1ccccc1O2]

Synthesized according to GP7 from 11-chloro-2-(trifluoromethyl)dibenzo[b,f][1,4]oxazepine (267 mg, 0.897 mmol) and homopiperazine (183 mg, 1.79 mmol). Purification of the crude product by flash chromatography (DCM/MeOH 95:5 to 9:1) yielded the title compound as a yellow solid (178 mg, 55%). ¹H NMR (400 MHz, CDCl₃) δ 7.69 (dd, J=8.6, 2.0, 1H), 7.63 (d, J=1.9 Hz, 1H), 7.36 (d, J=8.5 Hz, 1H), 7.13-7.06 (m, 3H), 7.99-6.95 (m, 1H), 3.98-3.13 (m, 9H), 2.14-1.90 (m, 2H).

### 3-((4-(2-Chlorodibenzo[b,f][1,4]oxazepin-11-yl) piperazin-1-yl)methyl)-1,2,4-oxadiazol-5 (4H)-one (Compound 15)

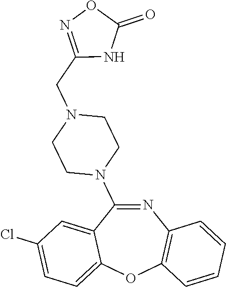 [SMILES: O=c1[nH]c(CN2CCN(C3=Nc4ccccc4Oc4ccc(Cl)cc43)CC2)no1]

To a solution of Compound 17 (22.0 mg, 0.0570 mmol) in EtOH (2.0 mL) a sodium methoxide solution in MeOH (5 M, 39.1 μL, 0.171 mmol) and diethyl carbonate (27.9 μL, 0.228 mmol) were added at rt and reaction mixture was heated to reflux overnight. Purification of the crude product by preparative HPLC yielded the title compound (11.0 mg, 47%). ¹H NMR (400 MHz, DMSO-d₆) δ 7.62 (dd, J=8.7, 2.6 Hz, 1H), 7.43 (d, J=2.4 Hz, 1H), 7.40 (d, J=9.6 Hz, 1H), 7.20-7.17 (m, 1H), 7.12-6.98 (m, 3H), 3.53-3.48 (m, 6H), 2.60 (br s, 4H). ¹³C{1H} (101 MHz, DMSO-d₆) δ 159.9, 158.7, 158.0, 157.4, 151.2, 139.9, 133.0, 129.5, 128.7, 126.5, 125.8, 124.4, 124.3, 123.1, 120.2, 51.9, 51.2, 46.6. MALDI-HRMS: m/z calculated for C₂₀H₁₉ClN₅O₃ [M+H]⁺: 412.1171, found: 412.1171.

### 2-(4-(2-Chlorodibenzo[b,f][1,4]oxazepin-11-yl) piperazin-1-yl) acetonitrile (Compound 16)

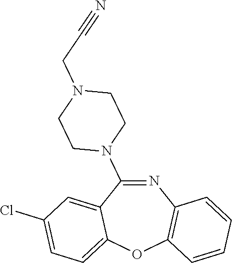 [SMILES: N#CCN1CCN(C2=Nc3ccccc3Oc3ccc(Cl)cc32)CC1]

Synthesized according to GP8 from Amoxapine (150 mg, 0.464 mmol), bromoacetonitrile (66.6 μL, 0.927 mmol), and triethylamine (653 μL, 4.64 mmol). Purification of the crude product by flash chromatography (DCM/MeOH 99:1 to 95:5) yielded the title compound (75.1 mg, 46%). ¹H NMR (400 MHz, CDCl₃) δ 7.39 (dd, J=8.5, 2.6 Hz, 1H), 7.32 (d, J=2.2 Hz, 1H), 7.20-7.15 (m, 2H), 7.12-7.06 (m, 2H), 7.03-6.98 (m, 1H), 3.59-3.51 (m, 6H), 2.71 (s, 4H). ¹³C{1H} (101 MHz, CDCl₃) δ 159.5, 159.0, 152.0, 139.6, 132.9, 130.5, 129.1, 127.2, 126.0, 125.1, 124.8, 122.9, 120.3, 114.6, 51.6, 47.2, 46.1. MALDI-HRMS: m/z calculated for C₁₉H₁₈ClN₄O [M+H]⁺: 353.1164, found: 353.1165.

### 2-(4-(2-Chlorodibenzo[b,f][1,4]oxazepin-11-yl) piperazin-1-yl)-N′-hydroxyacetimidamide (Compound 17)

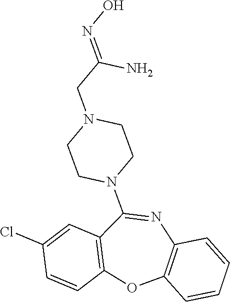 [SMILES: N/C(CN1CCN(C2=Nc3ccccc3Oc3ccc(Cl)cc32)CC1)=N\O]

To a solution of Compound 16 (50.0 mg, 0.140 mmol) in EtOH (1.0 mL) an aq hydroxylamine solution (50 wt %, 23.2 μL, 0.379 mmol) was added at rt and reaction mixture was heated to reflux overnight. Purification of the crude product by flash chromatography (DCM/MeOH 99:1 to 95:5) yielded the title compound (35.4 mg, 65%). ¹H NMR (400 MHz, DMSO-d6) δ 9.07 (br s, 1H), 7.61 (dd, J=8.7, 2.6 Hz, 1H), 7.41-7.37 (m, 2H), 7.20-7.17 (m, 1H), 7.10-6.98 (m, 3H), 5.30 (s, 2H), 3.47 (br s, 4H), 2.92 (s, 2H), 2.54-2.46 (m, 4H). ¹³C{1H} (101 MHz, DMSO-d₆) δ 158.6, 158.1, 151.2, 149.9, 139.9, 132.9, 129.5, 128.7, 126.5, 125.8, 124.5, 124.2, 123.1, 120.2, 57.8, 52.1, 46.8. MALDI-HRMS: m/z calculated for C₁₉H₂₁ClN₅O₂ [M+H]⁺: 386.1378, found: 386.1371.

### 5-((4-(8-Fluoro-2-(trifluoromethyl)dibenzo[b,f][1,4]oxazepin-11-yl) piperazin-1-yl)methyl)-1,2-dihydro-3H-1,2,4-triazol-3-one (Compound 18)

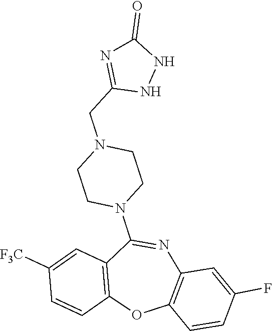 [SMILES: O=c1nc(CN2CCN(C3=Nc4cc(F)ccc4Oc4ccc(C(F)(F)F)cc43)CC2)[nH][nH]1]

Synthesized according to GP8 from Compound 187 (36.0 mg, 0.0985 mmol), 3-chloromethyl)-1H-1,2,4-triazol-5 (4H)-one (27.1 mg, 0.197 mmol), and triethylamine (137 μL, 0.985 mmol). Purification of the crude product by preparative HPLC yielded the title compound (13.2 mg, 29%). ¹H NMR (500 MHz, DMSO-d₆) δ 11.36 (s, 1H), 11.26 (s, 1H), 7.97 (dd, J=8.7, 2.1 Hz, 1H), 7.72 (d, J=2.1 Hz, 1H), 7.61 (d, J=8.5 Hz, 1H), 7.28-7.24 (m, 1H), 6.87-6.80 (m, 2H), 3.49 (br s, 4H), 3.36 (s, 2H), 2.60-2.44 (m, 4H). ¹³C{1H} (126 MHz, DMSO-d₆) δ 162.5, 160.7, 158.7, 158.5, 156.2, 147.1, 144.3, 141.48, 128.7, 126.3, 124.6, 123.3, 122.5, 121.4, 112.3, 110.3, 53.2, 51.9, 45.7. MALDI-HRMS: m/z calculated for C₂₁H₁₉F₄N₆O₂ [M+H]⁺: 463.1500, found: 463.1499.

### 5-((4-(2-Chloro-8-fluorodibenzo[b,f][1,4]oxazepin-11-yl) piperazin-1-yl)methyl)-1,2-dihydro-3H-1,2,4-triazol-3-one (Compound 19)

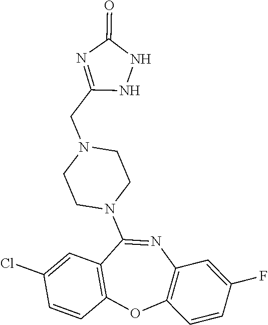 [SMILES: O=c1nc(CN2CCN(C3=Nc4cc(F)ccc4Oc4ccc(Cl)cc43)CC2)[nH][nH]1]

Synthesized according to GP8 from Compound 175 (50.0 mg, 0.151 mmol), 3-chloromethyl)-1H-1,2,4-triazol-5 (4H)-one (41.5 mg, 0.115 mmol), and triethylamine (210 μL, 1.51 mmol). Purification of the crude product by preparative HPLC yielded the title compound (38.9 mg, 60%). ¹H NMR (400 MHz, DMSO-d₆) δ 11.38 (s, 1H), 11.26 (s, 1H), 7.44-7.16 (m, 5H), 6.83-6.76 (m, 2H), 3.56 (s, 4H), 3.35 (s, 2H), 2.59-2.41 (m, 4H). ¹³C{1H} (101 MHz, DMSO-d₆) δ 160.8, 158.5, 158.4, 156.2, 147.5, 144.3, 141.8, 133.1, 129.6, 128.8, 124.2, 123.0, 121.6, 112.2, 110.1, 53.2, 52.0, 46.5. MALDI-HRMS: m/z calculated for C₂₀H₁₉ClFN₆O₂ [M+H]⁺: 429.1237, found: 429.1239.

### 5-((4-(2-(Trifluoromethyl)dibenzo[b,f][1,4]oxazepin-11-yl)-1,4-diazepan-1-yl)methyl)-1,2-dihydro-3H-1,2,4-triazol-3-one (Compound 20)

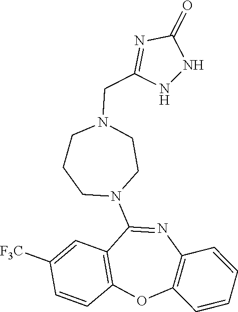 [SMILES: O=c1nc(CN2CCCN(C3=Nc4ccccc4Oc4ccc(C(F)(F)F)cc43)CC2)[nH][nH]1]

Synthesized according to GP8 from Compound 14 (20.0 mg, 0.0553 mmol), 3-chloromethyl)-1H-1,2,4-triazol-5 (4H)-one (15.2 mg, 0.111 mmol), and triethylamine (77.9 μL, 0.553 mmol). Purification of the crude product by flash chromatography (DCM/MeOH 95:5 to 8:2) yielded the title compound (16.8 mg, 67%). ¹H NMR (500 MHz, DMSO-d₆) δ 11.29 (s, 1H), 11.20 (s, 1H), 7.91 (dd, J=8.6, 2.1 Hz, 1H), 7.77 (d, J=2.0 Hz, 1H), 7.58 (d, J=8.5 Hz, 1H), 7.20 (dd, J=8.0, 1.4 Hz, 1H), 7.08-7.01 (m, 2H), 6.96-6.92 (m, 1H), 3.60 (s, 4H), 3.44 (s, 2H), 2.80-2.64 (m, 4H), 1.98-1.75 (m, 2H). ¹³C{1H} (126 MHz, DMSO-d₆) δ 162.2, 157.7, 156.2, 150.6, 145.2, 140.6, 129.7, 126.7, 126.4, 126.0, 125.7, 123.9, 123.7, 123.3, 122.3, 120.2. MALDI-HRMS: m/z calculated for C₂₂H₂₂F₃N₆O₂ [M+H]⁺: 459.1751, found: 459.1752.

### 2-(4-(8-Fluoro-2-(trifluoromethyl)dibenzo[b,f][1,4]oxazepin-11-yl) piperazin-1-yl) ethan-1-ol (Compound 21)

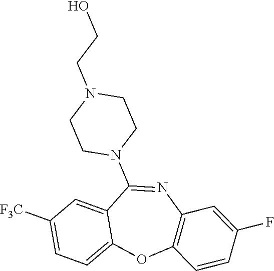 [SMILES: OCCN1CCN(C2=Nc3cc(F)ccc3Oc3ccc(C(F)(F)F)cc32)CC1]

Synthesized according to GP7 from Compound 232 (42.0 mg, 0.133 mmol) and 1-(2-hydroxyethyl) piperazine (33.3 μL, 0.266 mmol). Purification of the crude product by flash chromatography (DCM/MeOH 99:1 to 95:5) yielded the title compound (24.4 mg, 44%). ¹H NMR (400 MHz, CDCl₃) δ 7.72 (dd, J=8.5, 1.8 Hz, 1H), 7.61 (d, J=2.3 Hz, 1H), 7.36 (d, J=8.8 Hz, 1H), 7.07-7.04 (m, 1H), 6.85-6.82 (m, 1H), 6.71-6.67 (m, 1H), 3.62 (br s, 4H), 3.17-2.93 (m, 5H). ¹³C{1H} (101 MHz, CDCl₃) δ 163.2, 160.5, 159.4, 147.7, 141.4, 129.9, 127.7, 127.1, 123.6, 124.1, 122.2, 120.9, 113.4, 110.4, 59.5, 57.9, 52.8, 47.5. MALDI-HRMS: m/z calculated for C₂₀H₂₀F₄N₃O₂ [M+H]⁺: 410.1486, found: 410.1485

### Methyl 5-chloro-2-(2-nitrophenoxy)benzoate (Compound 22)

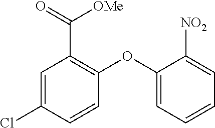 [SMILES: COC(=O)c1cc(Cl)ccc1Oc1ccccc1[N+](=O)[O-]]

Synthesized according to GP1 from methyl 5-chlorosalicylate (2.80 g, 15.0 mmol), 2-fluoronitrobenzene (1.06 mL, 10.0 mmol) and potassium carbonate (2.07 g, 15 mmol). Purification of the crude product by flash chromatography (n-hexane/EtOAc 9:1 to 8:2) yielded the title compound as a colorless solid (3.07 g, 99%). ¹H NMR (250 MHz, DMSO-d6) δ 8.07 (dd, J=8.1, 1.7 Hz, 1H) 7.92 (d, J=2.7 Hz, 1H), 7.75 (dd, J=8.8, 2.8 Hz, 1H) 7.68-7.60 (m, 1H), 7.37-7.30 (m, 1H), 7.27 (d, J=8.8 Hz, 1H), 7.00 (d, J=8.4, 1.1 Hz, 1H), 3.70 (s, 3H).

### Methyl 2-(2-nitrophenoxy)-5-(trifluoromethyl)benzoate (Compound 23)

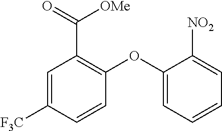 [SMILES: COC(=O)c1cc(C(F)(F)F)ccc1Oc1ccccc1[N+](=O)[O-]]

Synthesized according to GP1 from methyl 2-hydroxy-5-(trifluoromethyl)benzoate (991 mg, 4.50 mmol), 2-fluoronitrobenzene (317 μL, 3.00 mmol) and potassium carbonate (622 mg, 4.50 mmol). Purification of the crude product by flash chromatography (hexane/EtOAc 9:1 to 8:2) yielded the title compound as a colorless solid (615 mg, 60%). ¹H NMR (250 MHz, CDCl₃) δ 8.27 (s, 1H) 8.03 (d, J=8.2 Hz, 1H), 7.75 (d, J=8.6 Hz, 1H), 7.56 (t, J=8.2 Hz, 1H), 7.29 (t, J=8.2 Hz, 1H), 7.08 (d, J=8.6 Hz, 1H), 6.98 (d, J=8.3 Hz, 1H), 3.85 (s, 3H).

### Methyl 2-(2-aminophenoxy)-5-chlorobenzoate (Compound 24)

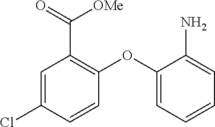 [SMILES: COC(=O)c1cc(Cl)ccc1Oc1ccccc1N]

Synthesized according to GP2 from methyl 5-chloro-2-(2-nitrophenoxy)benzoate (954 mg, 3.10 mmol) and tin (II) chloride dihydrate (2.80 g, 12.4 mmol). Purification of the crude product by flash chromatography (hexane/EtOAc 9:1 to 8:2) yielded the title compound as a colorless solid (635 mg, 74%). ¹H NMR (300 MHz, CDCl₃) δ 7.82 (d, J=2.6 Hz, 1H), 7.33 (dd, J=8.7, 2.6 Hz, 1H), 7.04-6.97 (m, 1H), 6.88-6.80 (m, 3H), 6.71 (td, J=7.7, 1.5 Hz, 1H), 3.89 (s, 3H), 3.64 (br s, 2H).

### Methyl 2-(2-aminophenoxy)-5-(trifluoromethyl)benzoate (Compound 25)

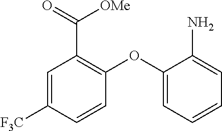 [SMILES: COC(=O)c1cc(C(F)(F)F)ccc1Oc1ccccc1N]

Synthesized according to GP2 from methyl 2-(2-nitrophenoxy)-5-(trifluoromethyl)benzoate (334 mg, 0.980 mmol) and tin (II) chloride dihydrate (900 mg, 3.91 mmol). Purification of the crude product by flash chromatography (hexane/EtOAc 9:1 to 8:2) yielded the title compound as a colorless solid (171 mg, 56%). ¹H NMR (250 MHz, CDCl₃) δ 8.12 (d, J=2.1 Hz, 1H), 7.61 (dd, J=8.7, 2.4 Hz, 1H), 7.10-7.02 (m, 1H), 6.98-6.93 (m, 2H), 6.84 (dd, J=7.9, 1.5 Hz, 1H), 6.79-6.72 (m, 1H), 3.99 (br s, 2H), 3.94 (s, 3H).

### 2-Chlorodibenzo[b,f][1,4]oxazepin-11 (10H)-one (Compound 26)

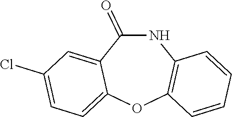 [SMILES: O=C1Nc2ccccc2Oc2ccc(Cl)cc21]

Synthesized according to GP3 from methyl 2-(2-aminophenoxy)-5-chlorobenzoate (630 mg, 2.27 mmol) and concentrated sulphuric acid (150 μL, 2.81 mmol). After filtration the title compound was obtained as a colorless solid (508 mg, 90%). ¹H NMR (250 MHz, DMSO-d₆) δ10.66 (s, 1H), 7.72 (d, J=2.5 Hz, 1H) 7.67 (dd, J=8.6, 2.8 Hz, 1H), 7.40 (d, J=8.6 Hz, 1H) 7.34 (dt, J=7.1, 1.2 Hz, 1H), 7.21-7.10 (m, 3H).

### 2-(Trifluoromethyl)dibenzo[b,f][1,4]oxazepin-11 (10H)-one (Compound 27)

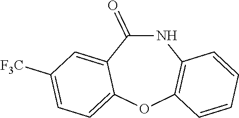 [SMILES: O=C1Nc2ccccc2Oc2ccc(C(F)(F)F)cc21]

Synthesized according to GP3 from methyl 2-(2-aminophenoxy)-5-(trifluoromethyl)benzoate (166 mg, 0.533 mmol) and concentrated sulphuric acid (35.3 μL, 0.663 mmol). After filtration the title compound was obtained as a colorless solid (73 mg, 49%). ¹H NMR (250 MHz, DMSO-d₆) δ 10.77 (s, 1H), 8.05-7.98 (m, 2H) 7.59 (d, J=8.3 Hz, 1H), 7.39 (dt, J=7.3, 1.2 Hz, 1H), 7.23-7.13 (m, 3H).

### 2-Fluoro-N-(5-fluoro-2-hydroxyphenyl)-5-(trifluoromethyl)benzamide (Compound 28)

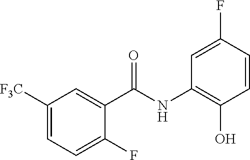 [SMILES: O=C(Nc1cc(F)ccc1O)c1cc(C(F)(F)F)ccc1F]

Synthesized according to GP4 from 2-fluoro-5-(trifluoromethyl)benzoic acid (1.50 g, 7.06 mmol), thionyl chloride (1.04 mL, 14.1 mmol), 2-amino-4-fluorophenol (925 mg, 7.06 mmol) and triethylamine (1.98 mL, 14.1 mmol). Purification of the crude product by flash chromatography (n-hexane/EtOAc 99:1 to 2:3) yielded the title compound as a red solid (1.43 g, 64%). ¹H NMR (400 MHz, DMSO-d₆) δ 8.17-8.13 (m, 1H), 8.05-7.91 (m, 2H), 7.66-7.59 (m, 1H), 6.93-6.80 (m, 2H).

### 8-Fluoro-2-(trifluoromethyl)dibenzo[b,f][1,4]oxazepin-11 (10H)-one (Compound 29)

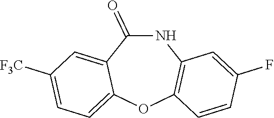 [SMILES: O=C1Nc2cc(F)ccc2Oc2ccc(C(F)(F)F)cc21]

Synthesized according to GP5 from 2-fluoro-N-(5-fluoro-2-hydroxyphenyl)-5-(trifluoromethyl)benzamide (196 mg, 0.618 mmol), and sodium hydroxide (25 mg, 0.618 mmol). After filtration the title compound was obtained as a light-brown solid (168 mg, 92%). ¹H NMR (250 MHz, DMSO-d₆) δ10.85 (s, 1H), 8.04-8.00 (m, 2H), 7.61-7.58 (m, 1H), 7.48-7.40 (m, 1H), 7.06-6.97 (m, 2H).

### 2,11-Dichlorodibenzo[b,f][1,4]oxazepane (Compound 30)

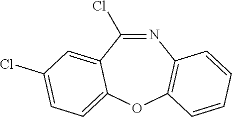 [SMILES: ClC1=Nc2ccccc2Oc2ccc(Cl)cc21]

Synthesized according to GP6 from 2-chlorodibenzo[b,f][1,4]oxazepin-11 (10H)-one (558 mg, 2.27 mmol), phosphorus oxychloride and N,N-dimethylaniline (173 μL, 1.36 mmol). After work up the title compound was obtained as brown solid (598 mg, 99%).

### 11-Chloro-2-(trifluoromethyl)dibenzo[b,f][1,4]oxazepine (Compound 32)

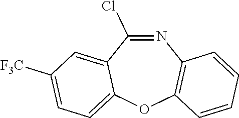 [SMILES: FC(F)(F)c1ccc2c(c1)C(Cl)=Nc1ccccc1O2]

Synthesized according to GP6 from 2-(trifluoromethyl)dibenzo[b,f][1,4]oxazepin-11 (10H)-one (73 mg, 0.261 mmol), phosphorus oxychloride and N,N-dimethylaniline (20.3 μL, 0.160 mmol). After work up the title compound was obtained as a brown solid (64 mg, 82%).

### 2,11-Dichlorodibenzo[b,f][1,4]thiazepine (Compound 33)

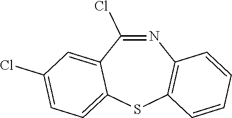 [SMILES: ClC1=Nc2ccccc2Sc2ccc(Cl)cc21]

Synthetized according to GP6 from commercially purchased 2-chlorodibenzo[b,f][1,4]thiazepin-11 (10H)-one (125 mg, 0.478 mmol), phosphorus oxychloride and N,N-dimethylaniline (36.8 μL, 0.290 mmol). After work up the title compound was obtained as a brown solid.

### 11-Chloro-8-fluoro-2-(trifluoromethyl)dibenzo[b,f][1,4]oxazepine (Compound 34)

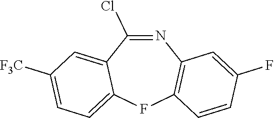

Synthetized according to GP6 from 8-fluoro-2-(trifluoromethyl)dibenzo[b,f][1,4]oxazepin-11 (10H)-one (120 mg, 0.404 mmol), phosphorus oxychloride and N,N-dimethylaniline (31.0 μL, 0.242 mmol). After work up the title compound was obtained as a light-brown solid.

### Methyl 5-chloro-2-(4-fluoro-2-nitrophenoxy)benzoate (Compound 35)

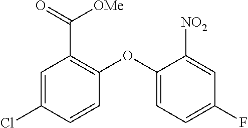 [SMILES: COC(=O)c1cc(Cl)ccc1Oc1ccc(F)cc1[N+](=O)[O-]]

Synthesized according to GP1 from methyl 5-chlorosalicylate (700 mg, 3.75 mmol), 2,5-difluoronitrobenzene (271 μL, 2.50 mmol) and potassium carbonate (524 mg, 3.75 mmol). Purification of the crude product by flash chromatography (n-hexane/EtOAc 9:1 to 8:2) yielded the title compound (811 mg, 99%). ¹H NMR (250 MHz, CDCl₃) δ 7.97 (d, J=2.7 Hz, 1H), 7.73 (dd, J=7.7, 3.1 Hz, 1H), 7.50 (dd, J=8.8, 2.7 Hz, 1H), 7.27-7.19 (m, 1H), 6.99 (d, J=8.7 Hz, 1H), 6.87 (dd, J=9.2, 4.4 Hz, 1H), 3.81 (s, 3H).

### Methyl 2-(2-amino-4-fluorophenoxy)-5-chlorobenzoate (Compound 36)

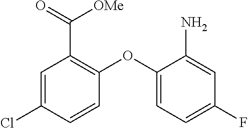 [SMILES: COC(=O)c1cc(Cl)ccc1Oc1ccc(F)cc1N]

Synthesized according to GP2 from Compound 35 (1.02 g, 3.14 mmol) and tin (II) chloride dihydrate (2.89 g, 12.6 mmol). Purification of the crude product by flash chromatography (n-hexane/EtOAc 9:1 to 8:2) yielded the title compound (630 mg, 68%). ¹H NMR (250 MHz, CDCl₃) δ 7.80 (d, J=2.7 Hz, 1H), 7.34 (dd, J=8.9, 2.7 Hz, 1H), 6.87-6.80 (m, 2H), 6.52 (dd, J=9.9, 2.9 Hz, 1H), 6.44-6.35 (m, 1H), 3.97 (br s, 2H), 3.91 (s, 3H).

### 2-Chloro-8-fluorodibenzo[b,f][1,4]oxazepin-11 (10H)-one (Compound 37)

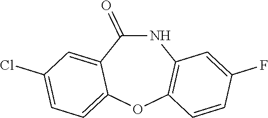 [SMILES: O=C1Nc2cc(F)ccc2Oc2ccc(Cl)cc21]

Synthesized according to GP31 from Compound 36 (625 mg, 2.11 mmol) and concentrated sulphuric acid (141 μL, 2.64 mmol). After work up the title compound was obtained (499 mg, 90%). ¹H NMR (250 MHz, DMSO-d₆) δ 10.73 (br s, 1H), 7.72 (d, J=2.3 Hz, 1H), 7.68 (dd, J=8.4, 1.4 Hz, 1H), 7.43-7.36 (m, 2H), 7.04-6.94 (m, 2H).

### 2,11-Dichloro-8-fluorodibenzo[b,f][1,4]oxazepine (Compound 38)

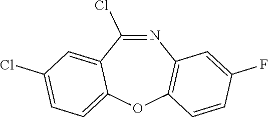 [SMILES: Fc1ccc2c(c1)N=C(Cl)c1cc(Cl)ccc1O2]

Synthesized according to GP6 from Compound 37 (150 mg, 0.569 mmol), phosphorus oxychloride and N,N-dimethylaniline (43.1 μL, 0.340 mmol). After work up the title compound was obtained (138 mg, 86%).

### 2-Chloro-8-fluoro-11-(piperazin-1-yl)dibenzo[b,f][1,4]oxazepine (Compound 39)

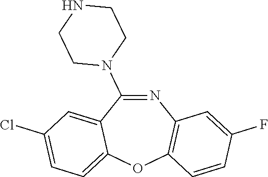 [SMILES: Fc1ccc2c(c1)N=C(N1CCNCC1)c1cc(Cl)ccc1O2]

Synthesized according to GP7 from Compound 38 (94.6 mg, 0.335 mmol) and piperazine (57.8 mg, 0.671 mmol). Purification of the crude product by flash chromatography (DCM/MeOH 99:1 to 8:2) yielded the title compound (74.1 mg, 67%). ¹H NMR (400 MHz, CDCl₃) δ 7.72 (dd, J=8.5, 1.8 Hz, 1H), 7.61 (d, J=2.3 Hz, 1H), 7.36 (d, J=8.8 Hz, 1H), 7.07-7.04 (m, 1H), 6.85-6.82 (m, 1H), 6.71-6.67 (m, 1H), 3.62 (br s, 4H), 3.17-2.93 (m, 5H). ¹³C{1H} (101 MHz, CDCl₃) δ 161.5, 159.2, 159.1, 148.0, 141.2, 133.1, 130.7, 129.0, 124.5, 122.8, 120.8, 120.7, 112.2, 46.7, 44.6. MALDI-HRMS: m/z calculated for C₁₇H₁₆ClFN₃ [M+H]⁺: 332.0960, found: 322.0959.

### 8-Fluoro-11-(piperazin-1-yl)-2-(trifluoromethyl)dibenzo[b,f][1,4]oxazepine (Compound 40)

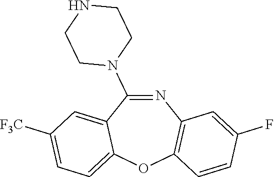 [SMILES: Fc1ccc2c(c1)N=C(N1CCNCC1)c1cc(C(F)(F)F)ccc1O2]

Synthesized according to GP7 from Compound 34 (128 mg, 0.404 mmol) and piperazine (69.6 mg, 0.808 mmol). Purification of the crude product by flash chromatography (DCM/MeOH 99:1 to 8:2) yielded the title compound (77.3 mg, 52%). ¹H NMR (400 MHz, CDCl₃) δ 7.72 (dd, J=8.5, 1.8 Hz, 1H), 7.61 (d, J=2.3 Hz, 1H), 7.36 (d, J=8.8 Hz, 1H), 7.07-7.04 (m, 1H), 6.85-6.82 (m, 1H), 6.71-6.67 (m, 1H), 3.62 (br s, 4H), 3.17-2.93 (m, 5H). ¹³C{1H} (101 MHz, CDCl₃) δ 163.3, 162.4, 159.4, 147.7, 141.4, 130.1, 127.8, 127.1, 124.7, 124.0, 122.5, 122.2, 120.9, 112.3, 45.3, 29.9. MALDI-HRMS: m/z calculated for C₁₈H₁₆F₄N₃O [M+H]⁺: 366.1224, found: 366.1226.

### 2-Fluoro-N-(5-fluoro-2-hydroxyphenyl)-5-(trifluoromethyl)benzamide (Compound 41)

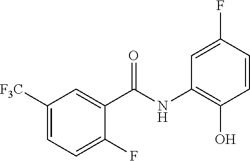 [SMILES: O=C(Nc1cc(F)ccc1O)c1cc(C(F)(F)F)ccc1F]

Synthesized according to GP4 from 2-fluoro-5-(trifluoromethyl)benzoic acid (1.50 g, 7.06 mmol), thionylchloride (1.0 mL, 14.1 mmol), 2-amino-4-fluorophenol (925 mg, 7.06 mmol), and triethylamine (2.0 mL, 14.1 mmol). Purification of the crude product by flash chromatography (n-hexane/EtOAc 99:1 to 4:6) yielded the title compound (1.43 g, 64%). ¹H NMR (250 MHz, CDCl₃) δ 8.15 (dd, J=6.4, 2.1 Hz, 1H), 8.05-7.98 (m, 1H), 7.94 (dd, J=10.4, 2.5 Hz, 1H), 7.63 (t, J=9.6 Hz, 1H), 6.94-6.80 (m, 2H).

### 8-Fluoro-2-(trifluoromethyl)dibenzo[b,f][1,4]oxazepin-11 (10H)-one (Compound 42)

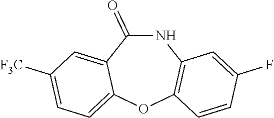 [SMILES: O=C1Nc2cc(F)ccc2Oc2ccc(C(F)(F)F)cc21]

Synthesized according to GP5 from Compound 41 (196 mg, 0.618 mmol) and sodium hydroxide (25 mg, 0.618 mmol). After work up the title compound was obtained (168 mg, 92%). ¹H NMR (250 MHz, DMSO-d₆) δ 10.85 (br s, 1H), 8.05-8.00 (m, 2H), 7.60 (d, J=8.1 Hz, 1H), 7.48-7.40 (m, 1H), 7.06-6.97 (m, 2H).

### 11-Chloro-8-fluoro-2-(trifluoromethyl)dibenzo[b,f][1,4]oxazepine (Compound 43)

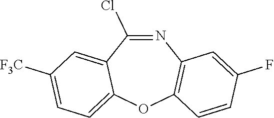 [SMILES: Fc1ccc2c(c1)N=C(Cl)c1cc(C(F)(F)F)ccc1O2]

Synthesized according to GP6 from Compound 42 (120 mg, 0.404 mmol), phosphorus oxychloride and N,N-dimethylaniline (31.0 μL, 0.242 mmol). After work up the title compound was obtained (50.4 mg, 39%).

### Example 3: Functional Activity Testing

Functional activity at human Slack was screened in vitro using a cell-based FluxOR assay in comparison to Loxapine as the reference activator. For that purpose, cultured HEK293 cells stably expressing human Slack (herein referred to as HEK-Slack cells) were incubated with compounds in a Na⁺ free buffer to prevent compound-independent Slack activation by monovalent cations^{7,19}.

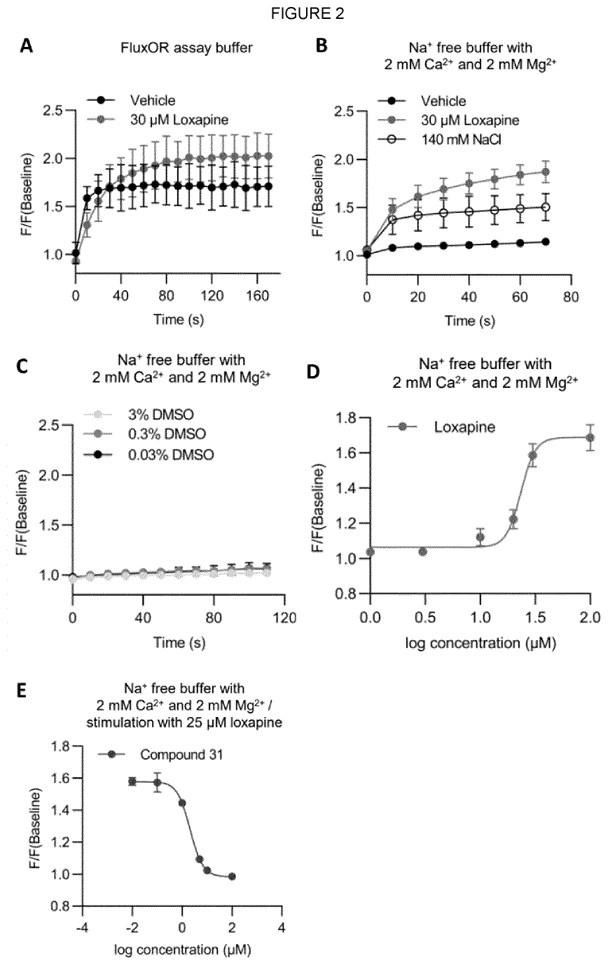

In first experiments, the inventors established that the modified FluxOR assay is capable to detect Slack activation by Loxapine (FIG. 2). Additionally, in dose-response experiments with the Slack inhibitor “Compound 31” (ref¹⁸)

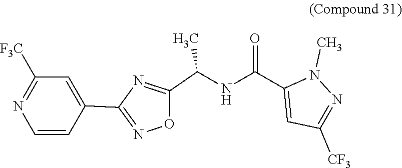 [SMILES: C[C@H](NC(=O)c1cc(C(F)(F)F)nn1C)c1nc(-c2ccnc(C(F)(F)F)c2)no1]

in a Na⁺ free buffer with 2 mM Ca²⁺, 2 mM Mg²⁺ and 0.03% DMSO and pre-stimulation with 25 μM Loxapine revealed that compound 31 inhibited the F/F (Baseline) ratio in a dose-dependent manner (IC₅₀=2.1 μM), confirming that the readout depends on Slack. Subsequently, dose-response curves were conducted to determine the EC₅₀ and E_{MAX} values.

In detail, in these experiments, the Slack-activating efficacy and potency of the novel compounds was determined using a commercial FluxOR™ Potassium Ion Channel Assay (Invitrogen). HEK293 cells stably expressing human Slack (herein referred to as HEK-Slack cells) were plated at a density of 50,000 cells per well of a poly-D-lysine coated (75 μg/mL, Sigma Aldrich) 96-well, black-walled microplate with clear bottom (Greiner Bio-One) in DMEM containing 10% FBS and 1% penicillin/streptomycin 24 hours prior to assaying.

On the experiment day, cells were loaded with FluxOR™ dye according to the manufacturer's protocol. A Na⁺-free assay buffer containing 140 mM choline chloride, 5 mM KCl, 2 mM CaCl₂), 2 mM MgSO₄, 10 mM HEPES, 5.55 mM glucose, adjusted to pH 7.4 with KOH, was used. To reduce background fluorescence all assay components were complemented by 10% BackDrop™ Background Suppressor (Invitrogen). To reduce background fluorescence all assay components were complemented by 10% BackDrop™ Background Suppressor (Invitrogen).

After washing steps, cells were incubated with compounds for 30 min at 37° C. All compounds were prepared as a 33.3 mM DMSO stock and diluted at the experimental day to a final concentration of 100, 50, 25, 12.5, 6.25, and 3.125 μM in assay buffer containing 0.03% DMSO. As a positive control 50 μM Loxapine was used. After stimulation with Tl₂SO₄ solution, the fluorescence was measured over 100 s (excitation 485 nm, emission 525 nm) using an Infinite M200 Microplate Reader (Tecan). Assays run in triplicate and the ratio to baseline fluorescence was calculated for all time points. Dose-response curves comprising six concentrations were performed in triplets using fluorescence/fluorescence_{Baseline} ratios at the 100^{th} second. Values were calculated relative to Loxapine within each individual experiment as follows: 100×((F/F_{B}(Compound)−F/F_{B}(Vehicleₘₑₐₙ)/(F/F_{B}(Loxapine)−F/F_{B}(Vehicleₘₑₐₙ)). To generate EC₅₀ and E_{MAX} values a standard, logistic, nonlinear regression analysis with GraphPad Prism 9.0 was used.

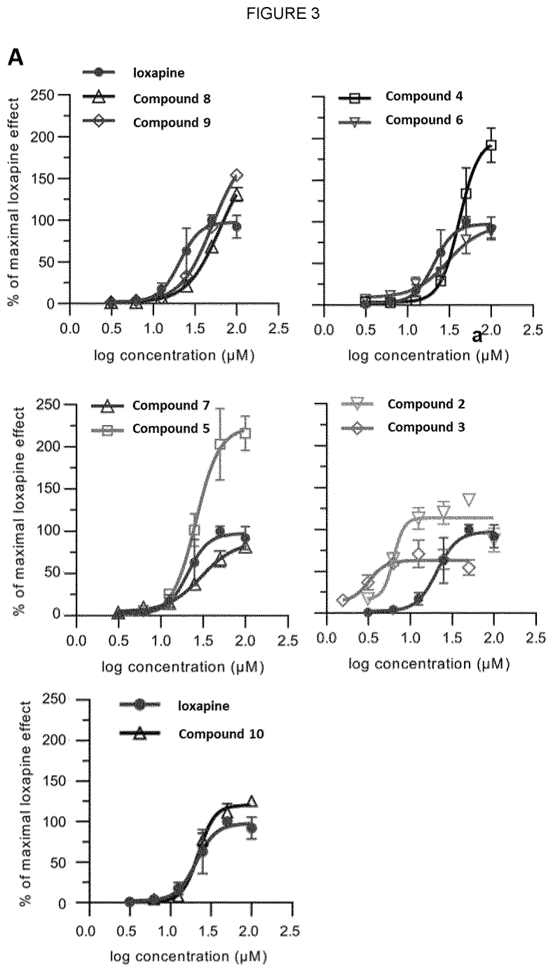

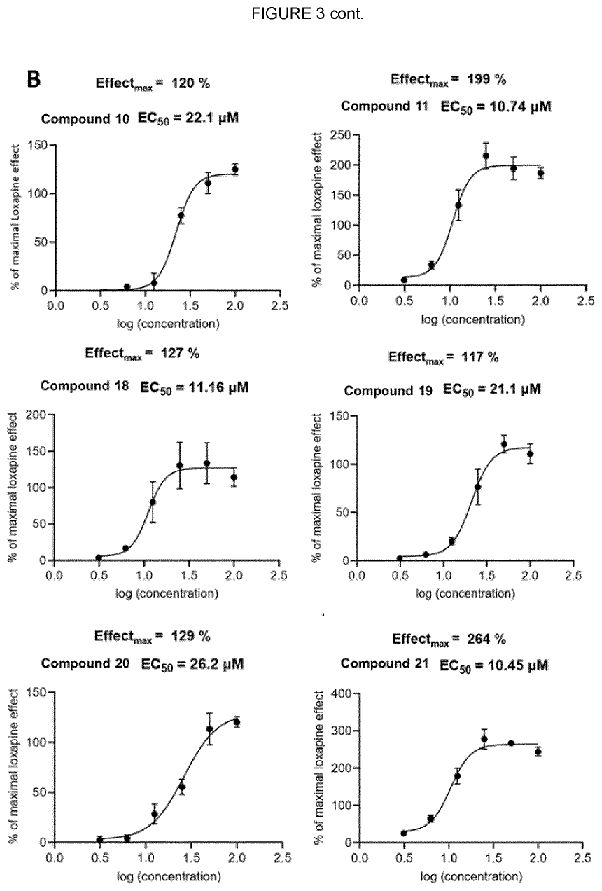

Out of 125 screened new compounds, 45 performed as Slack activators to a variable extent. The most interesting candidates exhibit a range of potencies between 3.2 and 62.5 μM, whereas Loxapine activated Slack with a potency of 20.7 μM (FIG. 3A, B and Table 3). Efficacy (E_{MAX}) values ranged from 63.1% to 264% as compared to Loxapine (FIG. 3A, B and Table 3). Considering that Loxapine at a low dose exerted Slack-dependent effects in mice¹⁹ and that the FluxOR assay was performed in a buffer containing 2 mM Ca²⁺ that inhibits Slack activity¹³ the inventors concluded that the new compounds might be sufficient to activate Slack at standard doses in vivo, albeit their EC₅₀ values are in the micromolar range. For compounds 10, 11 and 18-21, the corresponding time-dependent fluorescence/fluorescence_{Baseline} ratios are shown in FIG. 3E in comparison with Loxapine and vehicle.

TABLE 3
Structural features of the most promising new compounds and their Slack-
activating properties. Potency (EC₅₀) and efficacy (E_{MAX}; % relative to Loxapine)
values are from a FluxOR assay in HEK-Slack cells
that is shown in FIG. 3A, B. New compounds were measured in triplicate.
EC₅₀ and E_{MAX} values are presented as mean with 95% confidence interval.

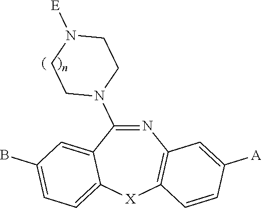

| | | | | | | FluxOR assay | |
|---|---|---|---|---|---|---|---|
| Sustituen | | | | | | EC₅₀ | E_{MAX} |
| Compound | B | E | A | X | n | μM | % |
| 1 (Loxapine) | Cl | 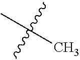 | H | O | 1 | 20.7 (16.3-26.4) | 100 |
| 2 | CF₃ | 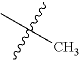 | H | O | 1 | 6.3 (5.3-7.4) | 114.3 (101.0-127.6) |
| 3 | CF₃ | 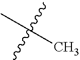 | H | O | 2 | 3.2 (2.3-4.5) | 63.1 (55.0-71.3) |
| 4 | CF₃ | 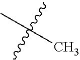 | F | O | 1 | 41.5 (36.2-47.6) | 199.1 (174.8-223.4) |
| 5 | Cl | 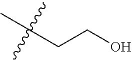 [SMILES: *CCO] | H | O | 1 | 26.0 (21.7-31.1) | 222.8 (193.6-252.0) |
| 6 | Cl | 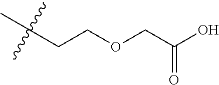 [SMILES: *CCOCC(=O)O] | H | O | 1 | 30.3 (20.8-44.1) | 96.2 (72.4-120.0) |
| 7 | Cl | 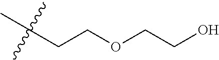 [SMILES: *CCOCCO] | H | S | 1 | 30.0 (23.9-37.7) | 87.4 (73.9-101.0) |
| 8 | CF₃ | 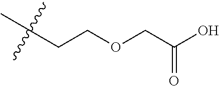 [SMILES: *CCOCC(=O)O] | H | O | 1 | 62.5 (49.3-79.2) | 177.0 (138.7-215.3) |
| 9 | CF₃ | 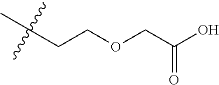 [SMILES: *CCOCC(=O)O] | H | O | 2 | 50.9 (46.0-56.3) | 186.4 (169.7-203.2) |
| 10 | Cl | 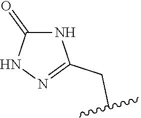 [SMILES: *Cc1n[nH]c(=O)[nH]1] | H | O | 1 | 22.1 (19.9-24.5) | 120.2 (112-128.4) |
| 11 | CF₃ | 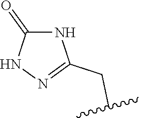 [SMILES: *Cc1n[nH]c(=O)[nH]1] | H | O | 1 | 10.74 (9.0-12.9) | 199.4 (184.7-214.2) |
| 18 | CF₃ | 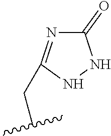 [SMILES: *Cc1nc(=O)[nH][nH]1] | F | O | 1 | 11.16 (8.4-14.9) | 126.8 (110.2-143.3) |
| 19 | Cl | 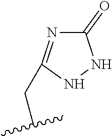 [SMILES: *Cc1nc(=O)[nH][nH]1] | F | O | 1 | 21.1 (18.1-24.5) | 117.4 (106.9-127.9) |
| 20 | CF₃ | 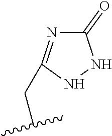 [SMILES: *Cc1nc(=O)[nH][nH]1] | H | O | 2 | 26.2 (21.0-32.8) | 128.8 (109.5-148.1) |
| 21 | CF₃ | 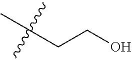 [SMILES: *CCO] | F | O | 1 | 10.45 (8.9-12.3) | 264.2 (247.9-280.4) |

When interpreting the EC₅₀ values obtained in the FluxOR assay, which are in the lower micromolar range, it must be taken into account that these experiments were performed with an assay buffer containing 2 mM Ca²⁺, which inhibits Slack activity. In such a setting, it is hardly possible to achieve Slack-activating EC₅₀ values in the lower nanomolar range. However, these values can still be used to estimate the efficacy of the compounds. As shown in further experiments on the inhibition of scratching behavior in mice after administration of Compound 6 and Compound 10 (compare example 9), the compounds with an EC50 as shown in Table 3 sufficiently activate Slack in vivo.

The evaluation of analogs in this series of Slack activators in the FluxOR assay suggests that replacement of the chloro substituent at B by a trifluoromethyl group improved the activity (Compound 2). Additional introduction of a fluoro substituent at the other aryl ring (A) increased the efficacy but reduced the potency (Compound 4). In some cases, sulfur analogues had comparable activity (Compound 7). Replacement of piperazine by homopiperazine improved the EC₅₀ value but decreased the E_{MAX} value (Compound 3). Modification of the alkyl residue at E was generally better tolerated and held potential for tuning of Slack activity and pharmacological properties. However, these conservative substituents did not change the highly lipophilic nature of the original scaffold, which presumably leads to CNS exposure similar to Loxapine. The substitution of the methyl group at the piperazine ring offered the opportunity for substantial increase of polarity. Derivatives with aliphatic hydroxy groups exhibited good efficacy with acceptable EC50 values (Compound 5). Finally, an ionizable carboxy group could be incorporated (Compound 6).

### Example 4: Slack Activation by the New Compounds was Verified Using Whole-Cell Patch-Clamp Recordings in HEK-Slack Cells

Generally, in these experiments, HEK-Slack cells were plated onto poly-D-lysine-coated (100 μg/mL, Sigma Aldrich) coverslips 1 day before experiments and cultured in DMEM containing 10% FBS and 1% penicillin/streptomycin in 5% CO₂ at 37° C. Whole-cell voltage clamp recordings were acquired using an EPC 9 amplifier combined with Patchmaster software (HEKA Electronics, Lambrecht/Pfalz, Germany). Currents were sampled at 20 kHz and filtered at 5 KHz. Data analysis was performed using the Fitmaster software (HEKA Electronics). Membrane potential was held at −70 mV and outward K⁺ current (I_{K}) was evoked by depolarizing steps (500 ms duration) ranging from −120 to +120 mV in increments of 20 mV. The pipette solution contained 140 mM KCl, 2 mM MgCl₂, 5 mM EGTA, 10 mM HEPES, and was adjusted to pH 7.4 with KOH. The extracellular solution contained 140 mM NaCl, 5 mM KCl, 2 mM CaCl₂), 2 mM MgCl₂, 10 mM HEPES, and was adjusted to pH 7.4 with NaOH. The osmolarity of all solutions was adjusted with glucose to 290-300 mOsmol/L. Patch pipettes had a resistance of 6-8 MΩ and were obtained from borosilicate glass capillaries (Science Products) using a conventional puller (DMZ-Universal Puller, Zeitz Instruments).

After baseline measurements, new compounds or Loxapine solved in external solution containing 0.03% DMSO (each with a final concentration of 50 μM) were added to the bath without a continuous perfusion, and K⁺ currents were measured within 5 min. Loxapine was used as a positive control whereas vehicle containing 0.03% DMSO served as a negative control. At a holding potential of −70 mV a series of 500 ms-long test pulses ranging from −120 to +120 mV in intervals of 20 mV were applied.

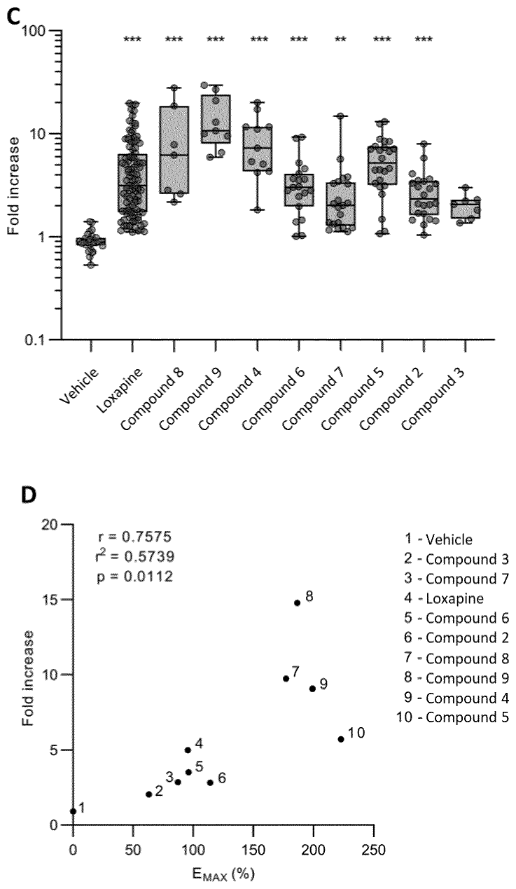

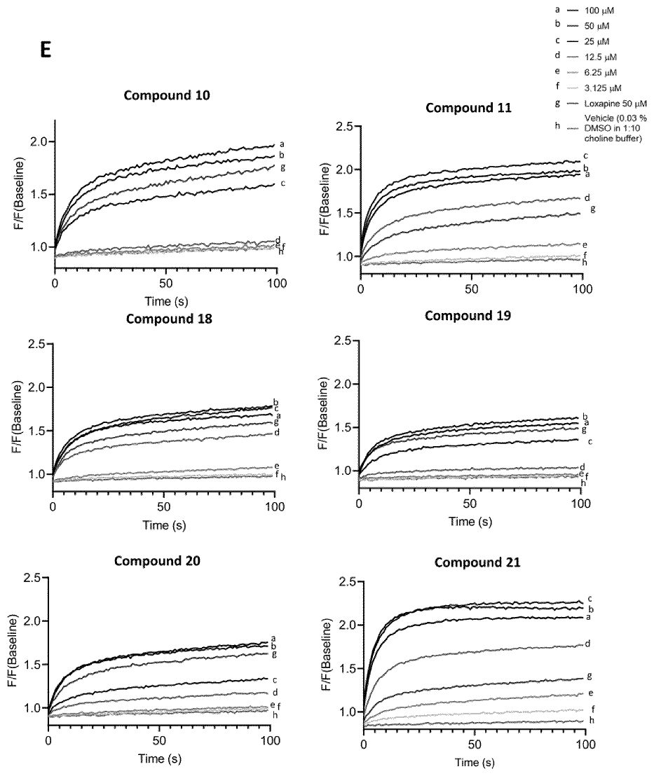

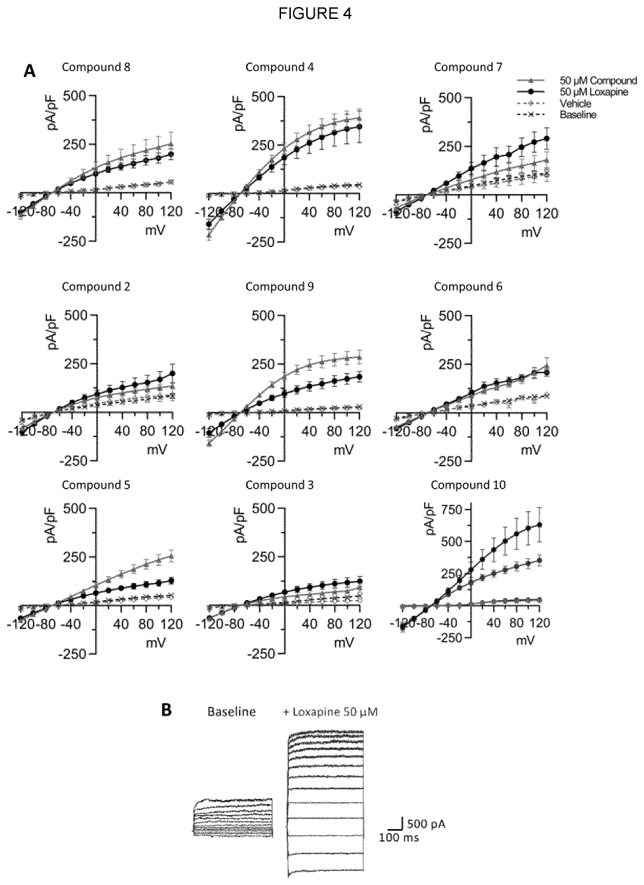

Fold increase values in patch-clamp experiments were determined by calculating baseline current density relative to current density after compound application. These patch-clamp recordings revealed that the new compounds (all except Compound 3) significantly increased the I_{K} amplitude in comparison to vehicle (FIG. 3C and FIG. 4A; representative I_{K} traces are shown in FIG. 4B).

At a voltage of +80 mV, current densities of new compounds were increased with a range between 2.04 to 14.77 fold compared to baseline (FIG. 3C). Notably, the efficacy of the new compounds in the patch-clamp recordings (i.e., the fold increase of current densities) significantly correlated with their efficacy in the FluxOR assay (FIG. 3D). In addition to the results shown in FIG. 3C and FIG. 4A, the following results were monitored in patch-clamp experiments for Compound 10 that were performed analogously as described before. For a Compound 10 concentration of 25 μM, a fold increase of 7.74±1.525 compared to baseline was monitored, for a Compound 10 concentration of 50 μM, a fold increase of 12.54±1.395 compared to baseline was monitored respectively.

Overall, the FluxOR assay and patch-clamp analyses in HEK-Slack cells indicate that the compounds according to the invention activate Slack in vitro.

### Example 5: Microsomal Stability Assay of Compound 10 in Liver Microsomes

For Compound 10, the metabolic stability in liver microsomes as shown in Table 4 was determined by the following assay. The test compound was dissolved in DMSO (1 mM). The solubilized test compound (5 μL, final concentration 10 μM) was preincubated at 37° C. in 432 μL of phosphate buffer (0.1 M, pH=7.4) together with 50 μL of NADPH regenerating system (30 mM glucose-6-phosphate, 4 U/mL glucose 6-phosphate dehydrogenase, 10 mM NADP, 30 mM MgCl2). After 5 min, the reaction was started by the addition of 13 μL of microsome mix from the liver of Sprague-Dawley rats (Invitrogen; 20 mg protein/mL in 0.1 M phosphate buffer) in a shaking water bath at 37° C. The reaction was stopped by adding 500 μL of ice-cold methanol at 0, 15, 30, and 60 min. The samples were centrifuged at 5000×g for 5 min at 4° C., and the test compound was quantified from the supernatants by HPLC: the composition of the mobile phase was adapted to the test compound in a range of MeOH 40-90% and water (0.1% formic acid) 10-60%. Flow rate: 1 mL/min; stationary phase: Purospher STAR, RP18, 5 μm, 125×4; precolumn: Purospher STAR, RP18, 5 μm, 4×4; detection wavelength: 254 and 280 nm; injection volume: 50 μL. Control samples were performed to check the test compound's stability in the reaction mixture. First control was without NADPH, which is needed for the enzymatic activity of the microsomes, second control was with inactivated microsomes (incubated for 20 min at 90° C.), and third control was without the test compound (to determine the baseline). The amounts of the test compound were quantified by an external calibration curve. Data are expressed as the mean±SEM remaining compound from three independent experiments. In vitro half-life was calculated by a logarithmic-linear transformation of the remaining amounts of nonmetabolized test compound versus time.

TABLE 4
Metabolic stability of Compound 10

| Time | |
|---|---|
| 15 min | 55% |
| 30 min | 35% |
| 60 min | 16% |

### Example 6: Blood-Brain-Barrier Specific Parallel Artificial Membrane Permeability Assay (PAMPA-BBB)

BBB permeability was estimated using a BBB-specific parallel artificial membrane permeability assay (PAMPA-BBB). PAMPA-BBB is a non-cell based permeation model that predicts the passive permeability of drug molecules through phospholipid membranes using a porcine polar brain lipid extract.²⁰

Following a slightly modified procedure as described by Müller et al.²¹, log Pₑ (effective permeability) values were determined for the prediction of the passive Blood-Brain-Barrier (BBB) permeability. Five v/v % DMSO in PBS (Phosphate Buffer Saline; 0.01 M, pH=7.4) buffer solutions of each compound were prepared with the final nominal concentration of 100 μM. These solutions were treated with ultrasonic waves and then centrifuged in case the test compounds were insoluble at this concentration. Proceeding with saturated, homogenous solutions (C_{D}(0)) is sufficient because log Pₑ only depends on relative concentrations.

A BBB specific PAMPA system was used to determine the log Pₑ and membrane retention (MR). Each well of the top plate (MultiScreen MAIPNTR10; Millipore; Billerica, US) was carefully coated with 5 μL of porcine polar brain lipid extract (PBLE; Avanti Polar Lipids, Birmingham, US) solution, then 150 μL of the C_{D}(0) solution was put on the membrane. The bottom plate (MultiScreen MDCPN2M50; Millipore, Billerica, US) was filled with 2050 μl PBS. PBLE solution consisted of 1 mg PBLE per 10 μL n-dodecane and 30 μL n-hexane. The donor plate was put on the acceptor plate and covered with a wet paper tissue and a plate lid. The sandwich system was incubated at 37° C. for 4 h. The plates were shaken and incubated in a Thermo Scientific™ MaxQ™ 4000 Benchtop Orbital Shaker (Gel, Belgium). After the incubation, PAMPA sandwich plates were separated and compound concentrations in donor (C_{D}(t)) and acceptor (C_{A}(t)) solutions were determined by UPLC-MS (Waters, Milford, US). The concentration of the donor solution at zero time point (C_{D}(0)) was determined using the supernatant after centrifugation. At iso-pH conditions, the effective permeability and the membrane retention of drugs were calculated by the following equation²²:

$${P}_{e} = \frac{- 2.303}{A \cdot ( t - {τ}_{SS} )} \cdot \frac{( {V}_{A} \cdot {V}_{D} )}{( {V}_{A} + {V}_{D} )} \cdot log ⌊ 1 - ( \frac{{V}_{A} + {V}_{D}}{( 1 - MR ) \cdot {V}_{D}} ) ( \frac{{C}_{A} ( t )}{{C}_{D} ( 0 )} ) ⌋ \qquad ( 1 )$$

where Pₑ is the effective permeability coefficient (cm/s), A is the filter area (0.3 cm²), V_{D} and V_{A} are the volumes in the donor (0.15 cm³) and acceptor phases (2.05 cm³), t is the incubation time(s), τ_{SS} is the time(s) to reach steady-state (240 s), C_{D}(t) is the concentration (mol/cm³) of the compound in the donor phase at time t, C_{A}(t) is the concentration (mol/cm³) of the compound in the acceptor phase at time t, C_{D}(0) is the concentration (mol/cm³) of the compound in the donor phase at time 0, and MR is the estimated membrane retention factor (the estimated mole fraction of solute lost to the membrane):

$$MR = ( 1 - \frac{{C}_{D} ( t )}{{C}_{D} ( 0 )} - \frac{{V}_{A}}{{V}_{D}} \cdot \frac{{C}_{A} ( t )}{{C}_{D} ( 0 )} ) \qquad ( 2 )$$

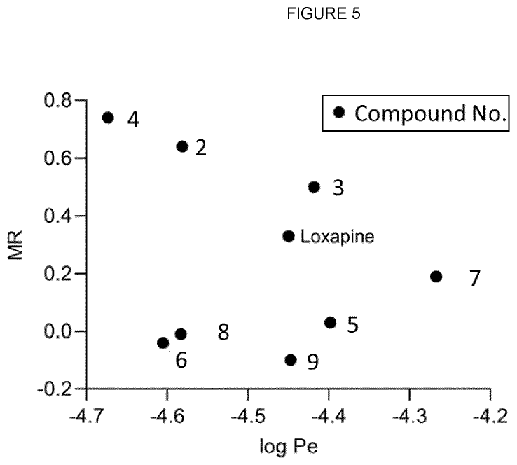

As shown in FIG. 5, the new compounds demonstrated variable effective BBB permeability (log Pₑ) values and membrane retention factors (MR; indicating the fraction of solute bound to the membrane). Evaluation of the set of compounds in this assay revealed that substitution at the aromatic core, introduction of the carboxylic acid linker and introduction of the hydroxy linker displayed little influence on the permeability. The missing decrease of passive permeability for the zwitterionic carboxylic acid derivatives might result from intramolecular charge neutralization, like it is known from cetirizine²³. On the contrary, replacement of piperazine by homopiperazine increased the permeability. Considering that a high MR factor limits the predictive validity of the corresponding log Pₑ value, the PAMPA-BBB in vitro data led the inventors to conclude that compounds Compound 6 and Compound 8 (with both log Pₑ<−4.57 and MR≤0) as well as Compound 10 (with log Pₑ=−4.492 and MR=0.02) most likely have a low BBB permeability.

TABLE 5
Brain permeability of the compounds of the
invention according to the PAMPA-BBB model.

PAMPA (compare FIG. 5)

| | | BBB permeability |
|---|---|---|
| Activators | MR | log Pₑ |
| Loxapine | 0.33 | −4.45 |
| Compound 8 | −0.01 | −4.58 |
| Compound 9 | −0.10 | −4.45 |
| Compound 4 | 0.74 | −4.67 |
| Compound 6 | −0.04 | −4.61 |
| Compound 7 | 0.19 | −4.27 |
| Compound 5 | 0.03 | −4.40 |
| Compound 2 | 0.64 | −4.58 |
| Compound 3 | 0.5 | −4.42 |
| Compound 10 | 0.02 | −4.492 |

### Example 7: Pharmacokinetic Properties of New Slack Activators by In Vivo Study

The compounds pharmacokinetic properties were examined in vivo by administering the compounds intraperitoneally (i.p.) at 1 mg/kg in mice, and determining plasma and brain levels over 8 h by LC-MS measurements.

The pharmacokinetic study was performed by Pharmacelsus (Saarbrücken, Germany). In total 27 male C57BL/6 mice (8 weeks old, 23-31 g body weight, purchased from Janvier Labs, France) were used. The animals were housed in a temperature-controlled room (20-24° C.) and maintained in a 12 h light/dark cycle. Food and water were available ad libitum. All experimental procedures were approved by and conducted in accordance with the regulations of the local Animal Welfare authorities (Landesamt für Gesundheit und Verbraucherschutz, Abteilung Lebensmittel-und Veterinärwesen, Saarbrücken).

The animals were divided into 3 groups (9 mice/group) and a cassette dosing was performed, in which the mice received a single dose of 1 mg/kg body weight of 3-4 compounds (dissolved in 0.9% NaCl containing 20% cyclodextrine) i.p. simultaneously. From each animal 2 blood samples were collected from the retrobulbar venous plexus under short isoflurane anesthesia (at 0.25 h and 0.5 h from 3 mice; at 1 h and 2 h from 3 mice; and at 4 h and 8 h from 3 mice). Li-heparin was used as anticoagulant. Plasma samples were obtained by centrifugation for 10 min at 3000× g and 4° C. Immediately after the last blood sample the animal was sacrificed by cervical dislocation and the brain was removed and frozen in liquid nitrogen. Plasma and brain samples were stored at −80° C. until LC-MS analysis.

LC-MS analysis was performed as follows: For Loxapine, Compound 2 and Compound 6, the HPLC system consisted of a Surveyor Pump Plus pump and a Surveyor Plus Auto sampler (Thermo Fisher Scientific, USA), and mass spectrometry was performed on a TSQ Quantum Discovery MAX mass spectrometer equipped with an ESI (electro spray ionization) interface (Thermo Fisher Scientific, USA) in positive SRM mode, connected to a PC running the standard software Xcalibur 2.0.7. For all other compounds, the HPLC system consisted of a U-HPLC pump (Accela) and an AS Open auto sampler (Thermo Fisher Scientific, USA), and mass spectrometry was performed on a Q Exactive (Orbitrap) accurate mass spectrometer equipped with a heated electrospray (H-ESI) interface (Thermo Fisher Scientific, USA) connected to a PC running the standard software Chromeleon 7.2. For all compounds, the HPLC pump flow rate was set to 600 μl/min and the compounds were separated on an analytical column with a suitable pre-column. The pharmacokinetic analysis was performed applying a non-compartment model using the Kinetica 5.0 software (Thermo Scientific, Waltham, USA). All given parameters were obtained by trapezoid area calculation.

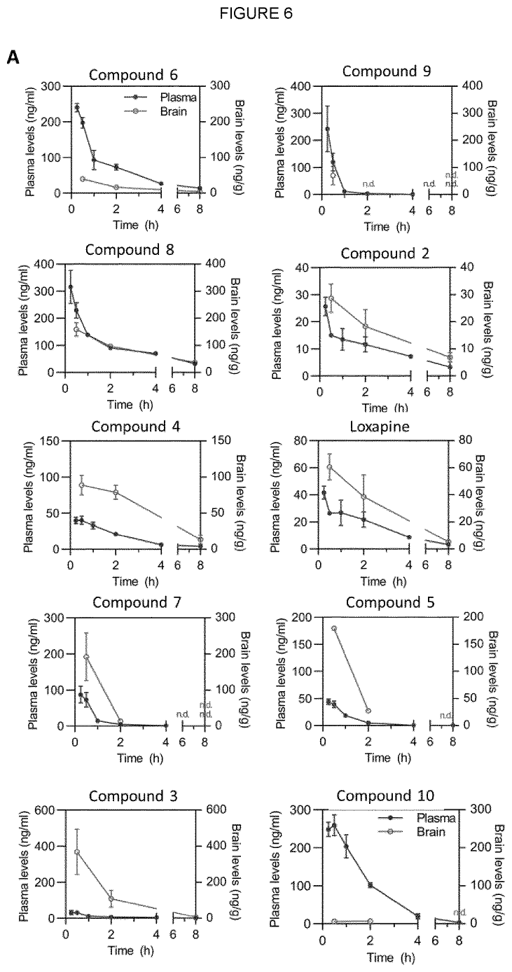

All compounds peaked in the plasma within 30 min of injection (FIG. 6A). Peak (Cₘₐₓ) plasma concentrations ranged from 25.6 to 315.5 ng/ml and the terminal elimination half-life (t_{1/2z}) ranged from 1.2 to 3.9 h (Table 6). In the brain, highest levels were also measured within 30 min of injection and ranged up to 369 ng/ml (FIG. 6A). The brain/plasma ratios are shown in FIG. 6B. Notably, Compound 6 and compound 10 have a low brain/plasma ratio of ≤0.3, suggesting that the brain penetration of these compounds is limited.

TABLE 6
Pharmacokinetic parameters of the new compounds in mice. Animals were i.p. injected
with 1 mg/kg of each compound and plasma and brain concentration at different time
points were measured by LC-MS analysis. Presented are pharmacokinetic parameters
based on plasma concentration. Time courses of plasma and brain concentration are
shown in FIG. 6A. The brain/plasma ratio is shown in FIG. 6B.

| Compound | | | | | | | | | |
|---|---|---|---|---|---|---|---|---|---|
| No. | 6 | 9 | 8 | 2 | 4 | Loxapine | 7 | 5 | 3 |
| Cmax (ng/ml) | 240.4 | 242.2 | 315.5 | 25.6 | 40.0 | 41.6 | 87.2 | 44.0 | 32.3 |
| tmax (h) | 0.25 | 0.25 | 0.25 | 0.25 | 0.5 | 0.25 | 0.25 | 0.25 | 0.5 |
| Cz (ng/ml) | 13.3 | 0.3 | 31.4 | 3.3 | 4.1 | 3.2 | 0.3 | 0.4 | 1.0 |
| tz (h) | 8 | 8 | 8 | 8 | 8 | 8 | 8 | 8 | 8 |
| t1/2z (h) | 2.4 | 1.8 | 3.9 | 3.4 | 2.3 | 2.3 | 1.6 | 1.2 | 2.0 |
| AUC(0-tz) | 419.7 | 122.6 | 675.1 | 67.8 | 108.2 | 105.2 | 71.2 | 49.6 | 55.4 |
| (ng*h/ml) | | | | | | | | | |
| AUC (0-inf) | 466.7 | 123.4 | 851.0 | 84.0 | 122.0 | 115.9 | 71.9 | 50.2 | 58.3 |
| (ng*h/ml) | | | | | | | | | |
| % AUCextra | 10.1 | 0.6 | 20.7 | 19.3 | 11.4 | 9.2 | 0.9 | 1.3 | 5.0 |
| Vz/f (ml/kg) | 7,560 | 21,080 | 6,582 | 58,489 | 27,724 | 28,837 | 31,440 | 33,111 | 50,117 |
| CL/f | 2,143 | 8,107 | 1,175 | 11,911 | 8,195 | 8,630 | 13,915 | 19,909 | 17,158 |
| (ml/(h*kg)) | | | | | | | | | |

### Example 8: Off Target Profile of Compound 6

As Loxapine is a first-generation antipsychotic with substantial binding affinity to many receptors²⁴ the inventors examined the pharmacological profile of Compound 6 and Loxapine (both 10 μM) in vitro using a SafetyScreen44 panel (Eurofins) that screens the interaction of compounds with 44 targets.

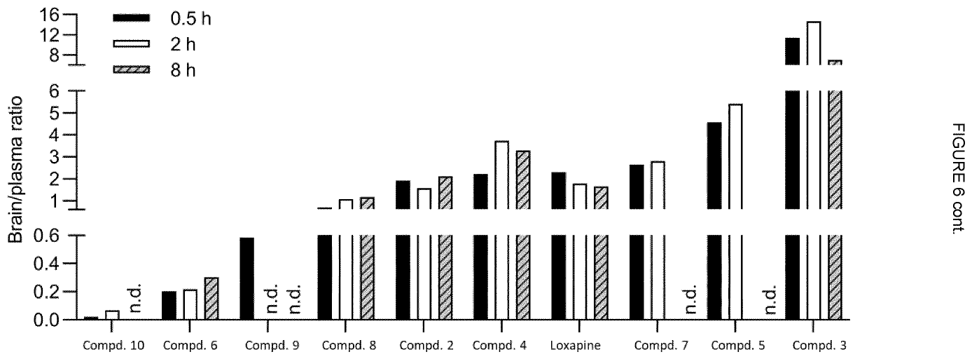

In this panel, Loxapine (10 μM) showed substantial binding inhibition (higher than 50%) for 17 out of 44 targets including adrenergic (α_{1A} and α_{2A}), dopamine (D₁ and D_{2S}), histamine (H₁ and H₂), muscarine (M₁, M₂ and M₃), serotonin (5-HT_{1A}, 5-HT_{1B}, 5-HT_{2A}, 5-HT_{2B} and 5-HT₃) receptors, Na⁺ channel, norepinephrine transporter (NET), and serotonin transporter (SET) (FIG. 7), thereby confirming the ‘dirty’ nature of first generation antipsychotics. Of note, considerably less off-target activities were observed with Compound 6 (10 μM), which exhibited substantial binding to 8 targets (α_{1A}, D₁, D_{2S}, H₁, 5-HT_{2A}, 5-HT_{2B}, NET, and SET), and with Compound 10 (10 μM), which showed substantial binding inhibition for 5 targets (CB₁, D₁, H₁, 5-HT_{2A}, and 5-HT_{2B}). Considering that the majority of the targets affected by Compound 6 or Compound 10 are mainly expressed in the CNS (in particular, D₁, D_{2S}, 5-HT_{2B}, NET, and SET^{25,26}; www.proteinatlas.org), and that both compounds show only limited brain penetration, these data suggest an improved pharmacological profile of Compound 6 and Compound 10 as compared to Loxapine. Furthermore, Compound 6 and Compound 10 (like Loxapine) did not measurably bind to human ether-à-go-go (hERG) or voltage-gated (K_{V}) potassium channels in the off-target screen (FIG. 7), indicating that this compound does not act as an unspecific modulator of potassium channels.

### Example 9: Compound 6 and Compound 10 Profoundly Inhibit Itch-Related Behavior in Mice

Further studies were conducted to assess the in vivo efficacy of Compound 6 and Compound 10 in mice. In detail, experiments were performed in 8 to 16 weeks old C57BL/6N mice (Charles River, Sulzfeld, Germany) and animals with a genetic deletion of Slack (Slack^{-/-} mice²⁷) of either sex. Animals were housed on a 12 h light/dark cycle with access to food and water ad libitum. All experiments adhered to the guidelines of the International Association for the Study of Pain and to the ARRIVE (Animal Research: Reporting on In Vivo Experiments) guidelines, and were approved by and conducted in accordance with the regulations of the local Animal Welfare authorities (Regierungspräsidium Darmstadt, Germany). All behavioral studies were conducted during the light cycle of the day at room temperature (20-24° C.) by an observer blinded for the treatment of the animals and/or their genotype.

Accelerating rotarod and vertical pole test were performed as follows: Mice were placed on a rotarod treadmill (Ugo Basile, Italy) with increasing speed (4-40 rpm over 300 s), and were trained for 4-5 consecutive days. Only mice reaching the 300 seconds without falling off were included in the experiment. On the testing day, compounds were i.p. administered in a vehicle composed of 0.9% saline (Sigma-Aldrich) containing 10% 2-hydroxypropyl-β-cyclodextrine (ITW Reagents) and after 15 min the latency to fall off the accelerating rotarod was measured. Immediately thereafter, mice were placed head-upward on the top of a vertical pole with a rough surface (diameter 1 cm, height 40 cm) and the time until animals reached the ground was recorded (cut-off time 20 s). Means out of three trials for each time point were calculated for further analysis.

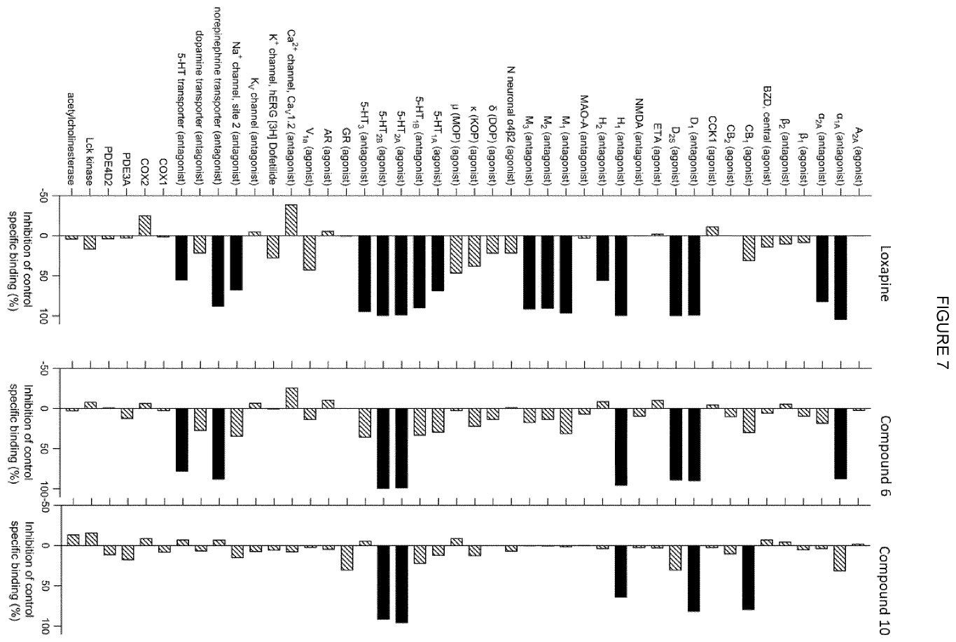

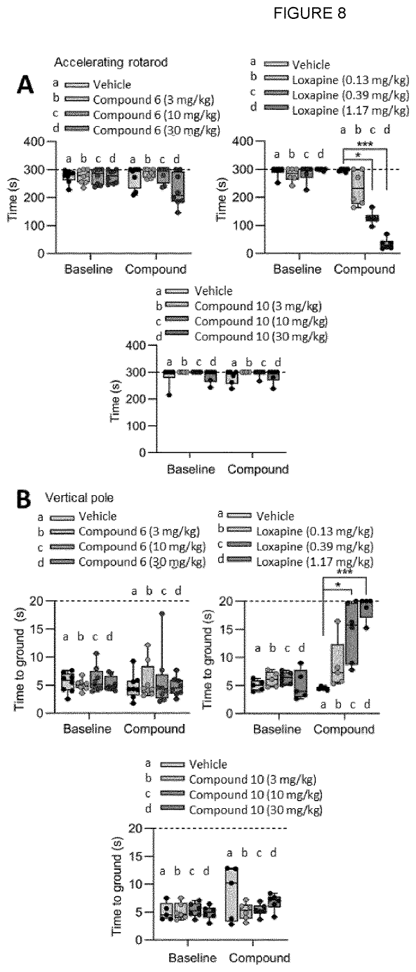

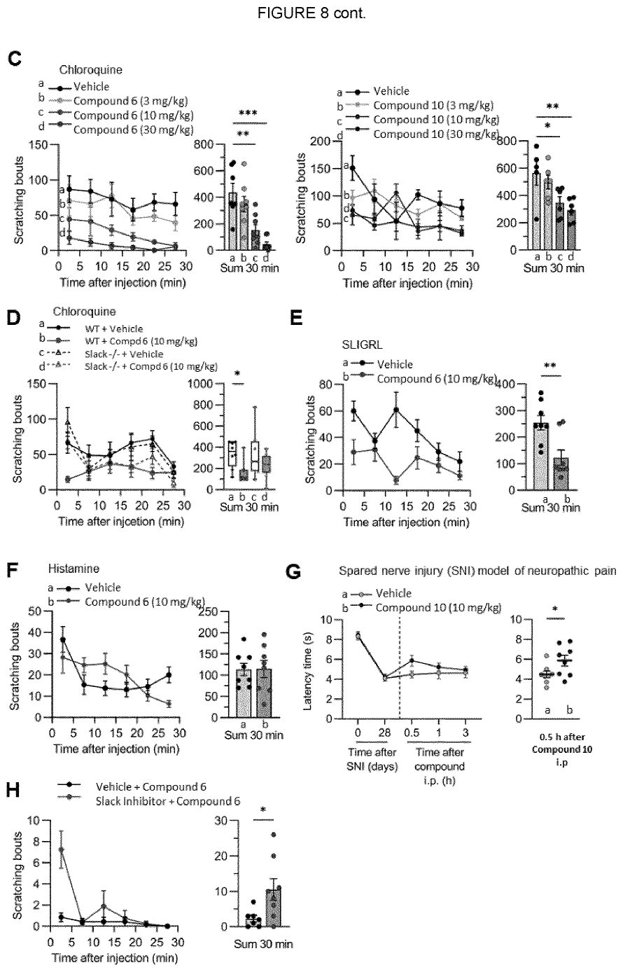

Treatments with Compound 6 and Compound 10 (3-30 mg i.p.) did neither impair the motor function in the accelerating rotarod test, a standard model of motor performance (FIG. 8A), nor in the vertical pole test that assesses basal ganglia related movement disorders (FIG. 8B). By contrast, Loxapine at a dose ≥0.39 mg/kg significantly reduced the ability of the mice to perform in both models (FIG. 8A,B), which is in line with its high CNS penetration and confirms earlier reports²⁸.

Acute itch behavior of the mice was monitored using a histamine-based, chloroquine-based and SLIGRL-based acute itch behavior model. In these models, three to four days before the experiment day, the fur on the dorsolateral aspect of the neck was shaved under brief isoflurane anesthesia. On the testing day, mice were habituated for 30 min in separate plexiglass cylinders (30 cm diameter). Compounds were dissolved in 0.9% saline containing 10% 2-hydroxypropyl-β-cyclodextrine and i.p. administered in a volume of maximal 200 μL. 15 min thereafter, the pruritogens chloroquine (200 μg), SLIGRL (100 μg) or histamine (800 μg), all dissolved in 0.9% saline (20 μL), were injected subcutaneously into the nape of the neck. Numbers of scratching bouts directed to the nape of the neck were assessed over 30 min by videotaping.

Antipruritic efficacy of Compound 6 and Compound 10 in a model of histamine-independent itch evoked by the antimalarial drug chloroquine: Importantly, a systemic treatment of the mice with Compound 6 (3, 10 or 30 mg/kg i.p.) or Compound 10 (3, 10 or 30 mg/kg i.p.) 15 min prior to s.c. injection of chloroquine into the nape of the neck was found to ameliorate the scratching behavior in a dose-dependent manner (FIG. 8C) in the histamine-independent itch-model, where itch was evoked by the antimalarial drug chloroquine. Further experiments supported the conclusion that the antipruritic effects were mediated by Slack activation, because Compound 6 (10 mg/kg) did not significantly alter chloroquine-induced scratching in animals with a genetic deletion of Slack (Slack^{-/-} mice; FIG. 8D). The assumption that Slack is the primary receptor mediating activity in vivo is supported by the observation that action of Compound 6 was much reduced in Slack−/− mice and blocked by Slack inhibitor Compound 31 (FIG. 8H).

Together, supported by genetic and pharmacological evidence these data suggest that Compound 6 or Compound 10 treatment ameliorates chloroquine-induced itch by activation of Slack.

Antipruritic efficacy of Compound 6 in histamine-independent itch evoked by SLIGRL injection: The chloroquine-induced scratching in mice is driven by activation of MrgprA3 in the NP2 population of sensory neurons^{4,29}. To further explore the antipruritic efficacy of Compound 6 in histamine-independent itch, the inventors assessed the behavioral response after s.c. injection of the peptide Ser-Leu-Ile-Gly-Arg-Leu (SLIGRL) that evokes scratching by activation of MrgprC11 (ref³⁰), which is expressed in the NP2 and NP3 population⁴. Similar to the chloroquine model, Compound 6 (10 mg/kg i.p.) significantly inhibited the SLIGRL-induced scratching behavior (FIG. 8E). By contrast, scratching evoked by s.c. histamine was not affected by Compound 6 (FIG. 8F), pointing to a limited function of Slack in the processing of histamine-dependent itch. Moreover, this finding provides further evidence that the antipruritic effects of Compound 6 observed in histamine-independent models are not the result of motor impairment.

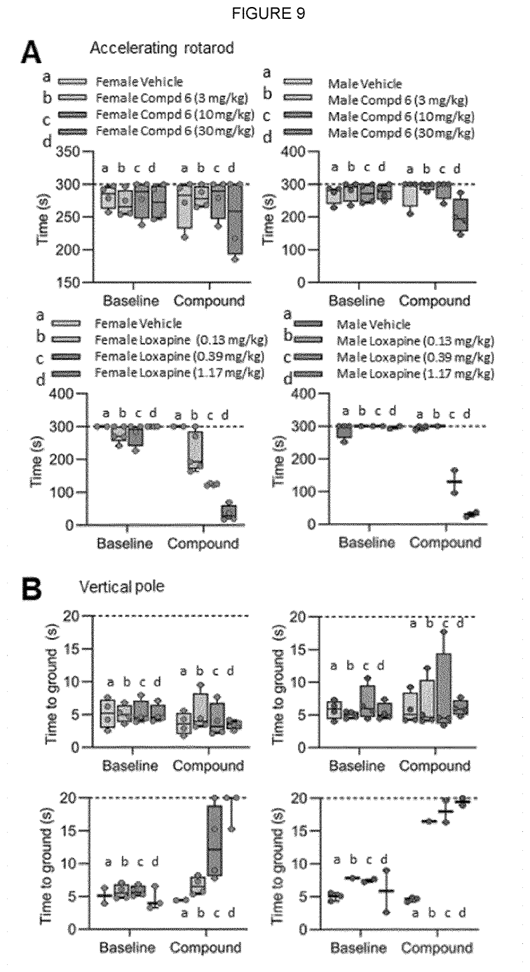

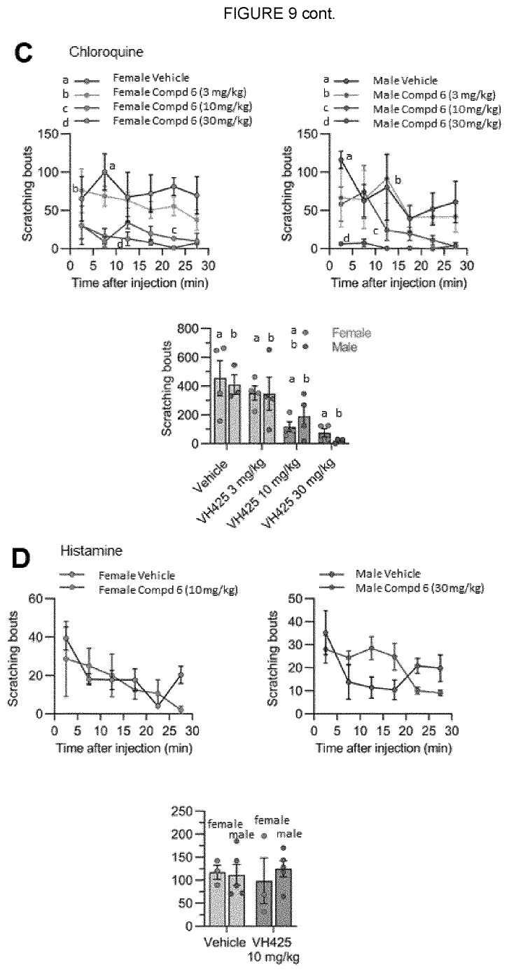

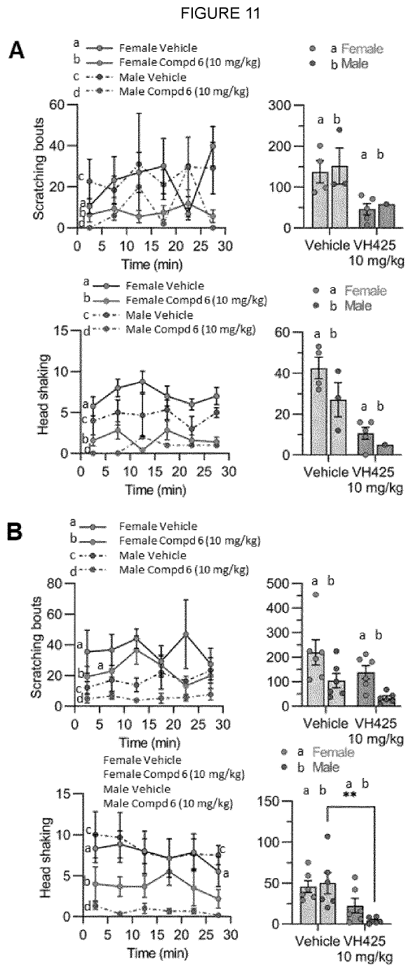

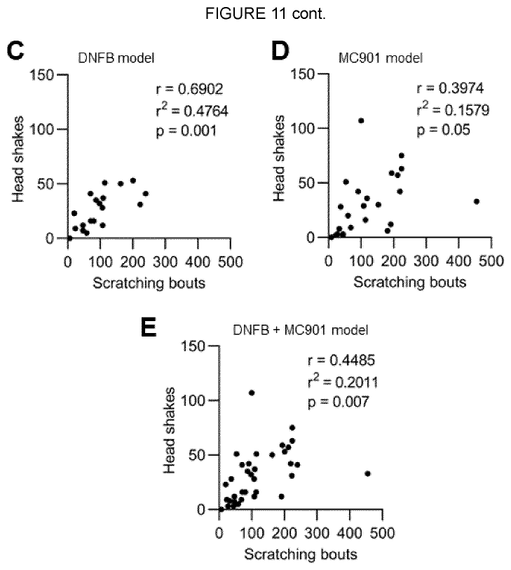

In these studies, no significant behavioral differences were observed between sexes (FIG. 9 and FIG. 11).

Furthermore, the effect of Compound 6 in chronic itch was examined in a model of allergic contact dermatitis³¹. In brief, the fur on the dorsolateral aspect of the neck was shaved under brief isoflurane anesthesia. Three to four days thereafter, the shaved skin area was painted with 100 μL of 0.15% 2,4-dinitrofluorobenzene (DNFB) in acetone/olive oil (3:1). After 10-11 days, the skin was shaved again and the DNFB solution was painted 3-4 days thereafter (i.e., two painting sessions 14 days apart). Ninety minutes after the second painting, mice were habituated for 15 min in separate plexiglass cylinders (30 cm diameter).

This treatment resulted in persistent itch behavior reflected by scratching the painted area and head-shaking that was significantly correlated with scratching.

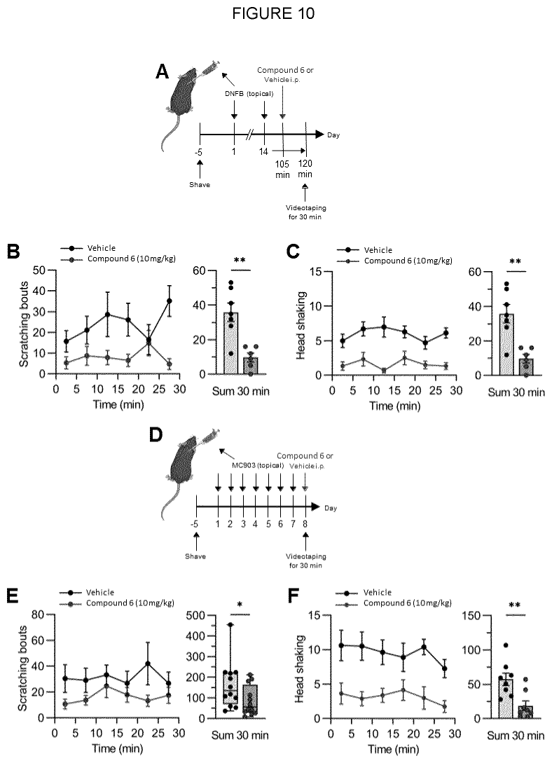

Compounds or vehicle (10% 2-hydroxypropyl-β-cyclodextrine in 0.9% saline) were i.p. administered, and 15 min thereafter spontaneous scratching was recorded over 30 min by videotaping (FIG. 10A). I.p. treatment with Compound 6 after the second DNFB exposure significantly inhibited the number of scratching bouts and the number of head shakes as compared to vehicle-treated animals (FIGS. 10B and 10C).

Similarly, the effect of Compound 6 was also examined in the MC903 model of skin inflammation, which has some characteristics of allergic contact dermatitis and atopic dermatitis³².

The fur on the dorsolateral aspect of the neck was shaved under brief isoflurane anesthesia. Five days thereafter, 20 μL MC903 (0.2 mM; Calcipotriol, Tocris) solved in absolute ethanol was applied to the skin at the back of the neck in brief anesthesia for 7 consecutive days. On day 8, mice were habituated for 30 min in separate plexiglass cylinders (30 cm diameter). Compounds or vehicle (10% 2-hydroxypropyl-β-cyclodextrine in 0.9% saline) were i.p. administered, and 15 min thereafter spontaneous scratching was recorded over 30 min by videotaping (FIG. 10D). In these studies, i.p. Compound 6 significantly ameliorated both scratching and head shaking (FIGS. 10E and 10F). These findings are important, because they indicate that Slack activators can ameliorate pruritus that has already established.

Finally, potential side effects of Compound 6 were assessed by measuring cardiovascular functions by pulse oximetry. A pulse oximeter (MouseOX Plus, Starr Life Sciences Corp) was used on conscious, freely moving mice. After 3 consecutive days of habituating the mice to the collar clip for at least 30 min, baseline measurements were performed. Then compounds were i.p. administered and after a resting time of 5 min the heart rate, respiratory rate and arterial O₂ saturation was recorded over 30 min. All datasets were sampled at 1 Hz, error-corrected according to manufacturer's instructions and averaged over 300 s for each time point.

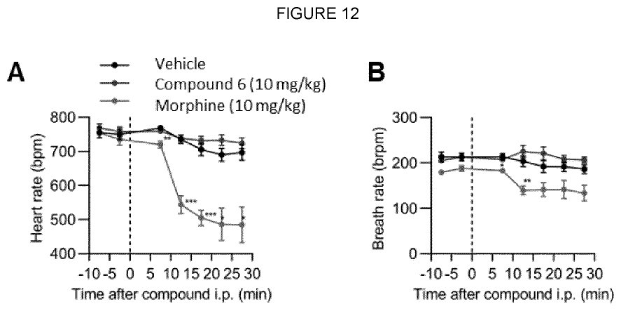

In these pulse oximetry measurements on conscious, freely moving mice i.p. administration of Compound 6 (10 mg/kg) did neither affect the heart rate (FIG. 12A) nor the breath rate (FIG. 12B) during an observation period of 30 min. By contrast, both parameters were significantly reduced after i.p. administration of morphine (10 mg/kg), which was used as a positive control in this experiment.

Altogether, these results highlight that Compound 6 effectively inhibits the itch behavior of mice in multiple models at a well-tolerated dose.

### Example 9: Compound 6 Inhibits Itch-Sensitive Sensory Neurons

To determine if the antipruritic effect of Compound 6 occurs directly at the neuronal level, whole-cell patch-clamp electrophysiology was used to measure excitability of DRG neurons.

A DRG neuron primary cell culture was prepared and stimulated with an inflammatory soup overnight to induce hyperexcitability. In brief, naïve C57BL/6N mice (age 4-8 weeks) were killed by CO₂ inhalation and lumbar (L1-L5) DRGs were transferred to hanks' balanced salt solution (Gibco, Thermo Fisher Scientific). After incubation with 500 U/mL collagenase IV and 2.5 U/ml dispase II (both from Sigma Aldrich) for 60 min, including carefully shaking every 20 min, DRGs were washed and gently triturated twice with a fire-polished Pasteur pipette in neurobasal medium (Gibco, Thermo Fisher Scientific) including 10% FBS and 0.5 mM GlutMax (Gibco, Thermo Fisher Scientific). Dissociated DRGs were seeded onto poly-D-lysine-coated (100 μg/mL) coverslips and cultured in neurobasal medium supplemented with 2% B27 (Gibco, Thermo Fisher Scientific), 1% penicillin/streptomycin and 0.5 mM GlutMax in 5% CO₂ at 37° C.

In order to simulate a pathological state, DRG neuronal cultures were incubated with an inflammatory soup (histamine: 10 μM, PGE₂: 10 μM, serotonin: 10 μM, bradykinin: 10 μM)³³ overnight and performed recordings on cells that bind isolectin B4, a marker of nonpeptidergic C-fiber neurons including the itch-sensitive neuronal populations (NP1-NP3)⁴ in mice. Before recording, DRG neurons were preincubated with 10 μg/mL FITC-conjugated IB4 (Sigma-Aldrich) for 5-10 min to select for Slack-expressing neurons.

Whole-cell current clamp recordings were obtained using an EPC 9 amplifier combined with Patchmaster software (HEKA Electronics, Lambrecht/Pfalz, Germany). General settings, pipette and extracellular solutions were used as described above. Evoked action potentials (AP) were elicited by 10 ms current injections starting at 0 pA in 20 pA increments to determine electrophysiological parameters. Action potential (AP) firing was induced by depolarizing current pulses (200-950 pA at 150 pA intervals, 1000 ms duration). Recordings were performed at baseline and following a 1 min incubation with 50 μM Compound 6 or vehicle (external solution containing 0.03% DMSO). Number of APs before and after compound application were counted at the same current injection step.

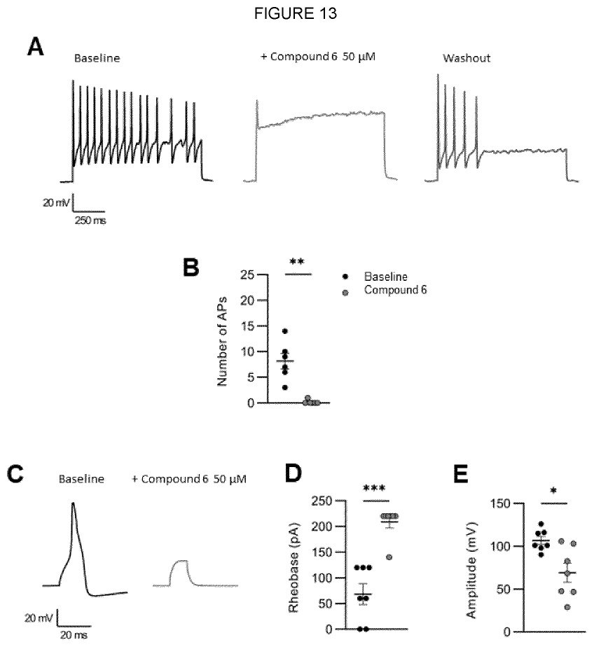

DRG neurons from the above-described cultures displayed pronounced hyperexcitability, as indicated by spontaneous action potential (AP) firing after injecting currents (200-950 pA; FIG. 13A). Notably, application of Compound 6 (50 μM) caused a profound decrease in the number of AP fired (FIG. 13B). Furthermore, after injection of small currents (0-220 pA), the rheobase (i.e., the amount of current required to generate an AP) was significantly increased in presence of Compound 6 (FIG. 13D) and the AP amplitude was significantly lower (FIG. 13E). These findings confirm that Compound 6 inhibits itch-sensitive DRG neurons in mice.

### Example 10: Chemicals/Drugs and Cell Cultures

Chemicals/Drugs: Loxapine, pregabalin, chloroquine, histamine, PGE₂, serotonin and bradykinin were purchased from Sigma-Aldrich. All indicated concentrations refer to pure substances. Reagents and solvents for the synthesis of Loxapine derivatives were obtained from Acros Organics (Gel, Belgium), Alfa Aesar GmbH & Co KG (Karlsruhe, Germany), BLDPharm Inc. (NuiNan, China), Fluorochem Ltd. (Hadfield, UK), Sigma-Aldrich (Munich, Germany), and TCI Europe N.V. (Zwijndrecht, Belgium).

Cell cultures: HEK293 cells stably transfected with human Kcnt1 (herein referred to as HEK-Slack cells; SB-HEK-KCa4.1; SB Drug Discovery, Lanarkshire, UK) were maintained in Dulbecco's modified Eagle's medium-Glutamax with 10% fetal calf serum and 1% penicillin/streptomycin, supplemented with 0.6 mg/ml G-418 (all from Gibco/Thermo Fisher Scientific) in 5% CO₂ at 37° C. Cells were passaged every 4 to 5 days from P11 to P35 depending on confluence.

Statistical analysis. Statistical analysis was performed in Prism 9 (GraphPad). Normally distributed data are expressed as the mean±standard error of the mean (SEM) and nonparametric data are presented as median and interquartile range. To identify and remove potential outliers the ROUT method with a Q (maximum desired false discovery rate) of 1% was used. The statistical test and number of replicates for each analysis is indicated in the figure legends or the main text. For all tests, a probability value P<0.05 was considered as statistically significant. No statistical methods were used to predetermine sample sizes.

### Example 11: Antinociceptive Effects of a Slack Activator (Compound 6) in a Mouse Model of Inflammatory Pain

Accumulating evidence indicates that the sodium-activated potassium channel Slack is highly expressed in a population of nociceptive sensory neurons that detect painful stimuli, and that Slack contributes to pain processing. Here, the inventors assessed whether systemic administration of a novel Slack activator, Compound 6, affects persistent inflammatory pain. Interestingly, in the mouse model of inflammatory pain evoked by intraplantar injection of complete Freund's (CFA) adjuvant, Compound 6 significantly ameliorated the CFA-induced paw hypersensitivity. These data suggest that Slack activators may prove to be an effective treatment of a variety of pain conditions in humans.

Animals: Experiments were performed in 8-16 week-old C57BL/6N mice (Charles River, Sulzfeld, Germany) of either sex. The animals were housed under a 12 h light/dark cycle with access to food and water ad libitum. All experiments adhered to the Animal Research: Reporting on In Vivo Experiments (ARRIVE) guidelines and were approved by and conducted in accordance with the regulations of the local Animal Welfare authorities (Regierungspräsidium Darmstadt, Germany; approval number V54-19c20/15-FR/2011). All behavioral studies were conducted during the light cycle of the day at room temperature (20-24° C.) by an observer blinded for the treatment of the animals and/or their genotype.

Behavioral assessment: Mechanical sensitivity of a hindpaw was assessed using a dynamic plantar aesthesiometer (Ugo Basile, Comerio, VA, Italy). Animals were placed on a wire mesh grid and habituated to the apparatus chamber for 1 h. A thin probe (0.5 mm diameter) was applied against the plantar surface of the paw from beneath with increasing force from 0 to 5 g within 10 s and a constant force of 5 g for additional 10 s until a strong withdrawal occurred. The paw withdrawal latency was recorded automatically and calculated as the average of 6-8 measurements. After baseline measurements, 20 μL of Complete Freund's adjuvant (CFA, containing 1 mg/mL heat-killed Mycobacterium tuberculosis in paraffin oil 85% and mannide monooleate 15%; Sigma-Aldrich, Darmstadt, Germany) was injected into the plantar surface of the hindpaw. Mechanical sensitivity was again determined 24 h after the CFA injection. Immediately thereafter, drug or vehicle (10% cyclodextrine in 0.9% NaCl) were intraperitoneally administered and the mechanical sensitivity was determined over 3 h.

Statistical Analysis: Statistical analysis was performed using Prism 9 (GraphPad). Kolmogorov-Smirnov tests were used to assess the normal distribution of data within groups. Normally distributed data were analyzed using two-way repeated measures ANOVA and Šídak's post-hoc test and are expressed as the mean±standard error of the mean (SEM). For all statistical tests, a probability value P<0.05 was considered statistically significant.

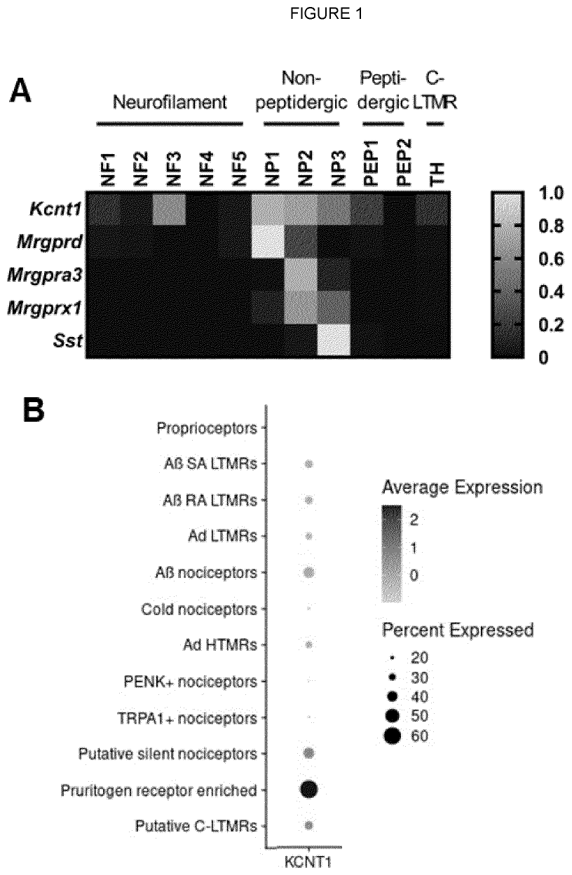

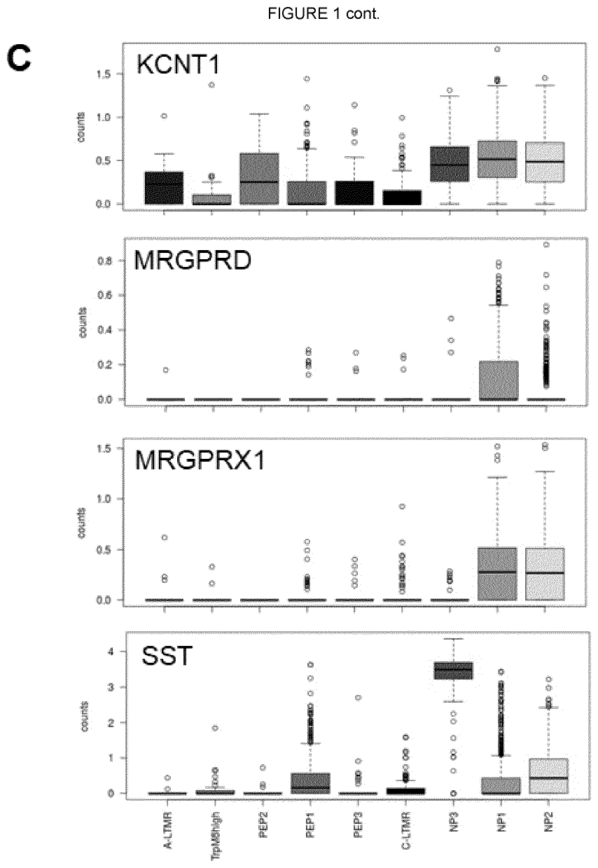

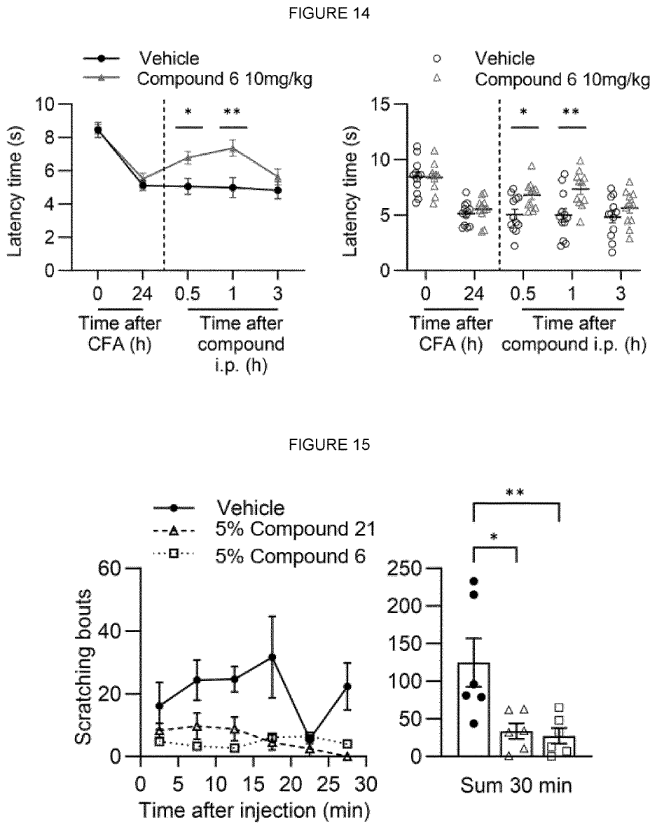

To investigate the antinociceptive effects of the Slack activator Compound 6 in vivo, the inventors used a murine model of inflammatory pain induced by intraplantar injection of CFA into a hindpaw, which induces a paw edema and hypersensitivity to mechanical stimuli. Results are shown in FIG. 14. Compound 6 (10 mg/kg) or vehicle were intraperitoneally administered 24 h after the CFA injection and the mice were evaluated for mechanical hypersensitivity by testing the latency to paw withdrawal using a dynamic plantar aesthesiometer. As shown in FIG. 1, the CFA injection evoked mechanical hypersensitivity, as indicated by a drop of paw withdrawal latencies 24 h post-CFA. Of note, 0.5 h and 1 h after administration of Compound 6 the paw withdrawal latencies were significantly increased compared to vehicle-treated animals, indicating an antinociceptive effect. These data suggest that persisting inflammatory pain can be ameliorated by Compound 6 in vivo.

### Example 12: Topical Application of Slack Activators Shows Efficacy in a Mouse Model of Pruritus

Various disorders are accompanied by histamine-independent (nonhistaminergic) itch that is often resistant to currently available therapies. Recently we have shown that pharmacological activation of Slack, a potassium channel highly expressed in itch-sensitive sensory neurons, holds therapeutic potential for treatment of itch. Previous studies revealed that intraperitoneal or oral administration of new Slack activators inhibited the scratching behavior in multiple models of acute histamine-independent and chronic itch without motor side effects in mice. Here, we investigated whether topical application of Slack-activating compounds shows antipruritic efficacy. Interestingly, topical application of two Slack activators, Compound 6 and Compound 21, inhibited the scratching behavior in a mouse model of chloroquine-induced, histamine-independent itch. These data suggest that topically administered Slack activators may prove to be an effective treatment of a variety of pruritic conditions in humans.

Animals: Experiments were performed in 8-16 week-old C57BL/6N mice (Charles River, Sulzfeld, Germany) of either sex. The animals were housed under a 12 h light/dark cycle with access to food and water ad libitum. All experiments adhered to the Animal Research: Reporting on In Vivo Experiments (ARRIVE) guidelines and were approved by and conducted in accordance with the regulations of the local Animal Welfare authorities (Regierungspräsidium Darmstadt, Germany; approval number V54-19c20/15-FR/2011). All behavioral studies were conducted during the light cycle of the day at room temperature (20-24° C.) by an observer blinded for the treatment of the animals.

Drug formulation: A creme formulation of Compound 6 and Compound 21 was prepared by mixing a hydrophilic creme (Basiscreme DAC) with 5% of each Slack activator.

Behavioral assessment: Three to four days before the day of the experiment, the fur on the dorsolateral aspect of the neck was shaved under brief isoflurane anesthesia. On the testing day, Compound 6 creme, Compound 21 creme, or creme vehicle were applied topically on the shaved region of the neck under brief isoflurane anesthesia and the mice were habituated to a Plexiglas cylinder (30 cm in diameter) for 30 min immediately thereafter. After habituation, the pruritogen chloroquine (200 μg; dissolved in 20 μL 0.9% saline) was injected subcutaneously into the nape of the neck. The number of scratching bouts directed towards the nape of the neck was assessed over 30 min by videotaping.

Statistical Analysis: Statistical analysis was performed using Prism 9 (GraphPad). Kolmogorov-Smirnov tests were used to assess the normal distribution of data within groups. Normally distributed data were analyzed using one-way-ANOVA and Dunnet's post-hoc test and are expressed as the mean±standard error of the mean (SEM). For all statistical tests, a probability value P<0.05 was considered statistically significant.

The inventors assessed the antipruritic efficacy of the Slack-activating compounds Compound 6 and Compound 21 in a model of histamine-independent itching induced by the antimalarial drug chloroquine. Importantly, we found that topical application of Compound 6 (5%) and Compound 21 (5%) 30 min prior to subcutaneous injection of chloroquine into the nape of the neck significantly ameliorated scratching behavior as compared to vehicle-treated animals (FIG. 15). These data suggest that topical treatment with Slack activators ameliorates histamine-independent itching.

### REFERENCES

The references are:
¹ Carstens, E., Follansbee, T. & Iodi Carstens, M. The Challenge of Basic Itch Research. Acta Derm. Venereol. 100, adv00023 (2020).
² Yosipovitch, G., Rosen, J. D. & Hashimoto, T. Itch: From mechanism to (novel) therapeutic approaches. J. Allergy Clin. Immunol. 142, 1375-1390 (2018).
³ Chen, X. J. & Sun, Y. G. Central circuit mechanisms of itch. Nat. Commun. 11, 3052 (2020).
⁴ Usoskin, D., et al. Unbiased classification of sensory neuron types by large-scale single-cell RNA sequencing. Nat. Neurosci. 18, 145-153 (2015).
⁵ Meixiong, J. & Dong, X. Mas-Related G Protein-Coupled Receptors and the Biology of Itch Sensation. Annu. Rev. Genet. 51, 103-121 (2017).
⁶ Tsantoulas, C. & McMahon, S. B. Opening paths to novel analgesics: the role of potassium channels in chronic pain. Trends Neurosci. 37, 146-158 (2014).
⁷ Yuan, A., et al. The sodium-activated potassium channel is encoded by a member of the Slo gene family. Neuron 37, 765-773 (2003).
⁸ Budelli, G., et al. SLO2 Channels Are Inhibited by All Divalent Cations That Activate SLO1 K+ Channels. J. Biol. Chem. 291, 7347-7356 (2016).
⁹ Sharma, N., et al. The emergence of transcriptional identity in somatosensory neurons. Nature 577, 392-398 (2020).
¹⁰ Martinez-Espinosa, P. L., et al. Knockout of Slo2.2 enhances itch, abolishes KNa current, and increases action potential firing frequency in DRG neurons. Elife 4 (2015).
¹¹ Kupari, J., et al. Single cell transcriptomics of primate sensory neurons identifies cell types associated with chronic pain. Nat. Commun. 12, 1510 (2021).
¹² Tavares-Ferreira, D., et al. Spatial transcriptomics of dorsal root ganglia identifies molecular signatures of human nociceptors. Sci. Transl. Med. 14, eabj8186 (2022).
¹³ Biton, B., et al. The antipsychotic drug Loxapine is an opener of the Na+-activated potassium channel Slack (slo2.2). J. Pharmacol. Exp. Ther. 340, 706-715 (2011).
¹⁴ Chakrabarti, A., et al. Loxapine for schizophrenia. Cochrane Database Syst. Rev., CD001943 (2007).
¹⁵ Schmiedl, S., et al. Loxapine for Treatment of Patients With Refractory, Chemotherapy-Induced Neuropathic Pain: A Prematurely Terminated Pilot Study Showing Efficacy But Limited Tolerability. Front Pharmacol 10, 838 (2019).
¹⁶ Tavares-Ferreira, D., et al. Spatial transcriptomics of dorsal root ganglia identifies molecular signatures of human nociceptors. Sci. Transl. Med. 14, eabj8186 (2022).
¹⁷ Kupari, J., et al. Single cell transcriptomics of primate sensory neurons identifies cell types associated with chronic pain. Nat. Commun. 12, 1510 (2021).
¹⁸ Griffin, A. M., et al. Discovery of the First Orally Available, Selective KNa1.1 Inhibitor: In Vitro and In Vivo Activity of an Oxadiazole Series. ACS Med Chem Lett 12, 593-602 (2021).
¹⁹ Lu, R., et al. Slack channels expressed in sensory neurons control neuropathic pain in mice. J. Neurosci. 35, 1125-1135 (2015).
²⁰ Avdeef, A. The rise of PAMPA. Expert Opin. Drug Metab. Toxicol. 1, 325-342 (2005).
²¹ Muller, J., Esso, K., Dargo, G., Konczol, A. & Balogh, G. T. Tuning the predictive capacity of the PAMPA-BBB model. Eur. J. Pharm. Sci. 79, 53-60 (2015).
²² Avdeef, A. Permeability-PAMPA. in Absorption and Drug Development: Solubility, Permeability, and Charge State, Second Edition 319-498 (Wiley, 2012).
²³ Pagliara, A., et al. Molecular properties and pharmacokinetic behavior of cetirizine, a zwitterionic H1-receptor antagonist. J. Med. Chem. 41, 853-863 (1998).
²⁴ Popovic, D., Nuss, P. & Vieta, E. Revisiting Loxapine: a systematic review. Ann Gen Psychiatry 14, 15 (2015).
²⁵ Sjostedt, E., et al. An atlas of the protein-coding genes in the human, pig, and mouse brain. Science 367 (2020).
²⁶ Karlsson, M., et al. A single-cell type transcriptomics map of human tissues. Sci Adv 7 (2021).
²⁷ Lu, R., et al. Slack channels expressed in sensory neurons control neuropathic pain in mice. J. Neurosci. 35, 1125-1135 (2015).
²⁸ Popovic, D., Nuss, P. & Vieta, E. Revisiting Loxapine: a systematic review. Ann Gen Psychiatry 14, 15 (2015).
²⁹ Liu, Q., et al. Sensory neuron-specific GPCR Mrgprs are itch receptors mediating chloroquine-induced pruritus. Cell 139, 1353-1365 (2009).
³⁰ Liu, Q., et al. The distinct roles of two GPCRs, MrgprC11 and PAR2, in itch and hyperalgesia. Sci. Signal. 4, ra45 (2011).
³¹ Kitamura, A., Takata, R., Aizawa, S., Watanabe, H. & Wada, T. A murine model of atopic dermatitis can be generated by painting the dorsal skin with hapten twice 14 days apart. Sci Rep 8, 5988 (2018).
³² Li, M., et al. Topical vitamin D3 and low-calcemic analogs induce thymic stromal lymphopoietin in mouse keratinocytes and trigger an atopic dermatitis. Proc. Natl. Acad. Sci. U.S.A. 103, 11736-11741 (2006).
³³ Grundy, L., et al. Chronic linaclotide treatment reduces colitis-induced neuroplasticity and reverses persistent bladder dysfunction. JCI Insight 3, e121841 (2018).

1. A compound, wherein the compound has the formula I: 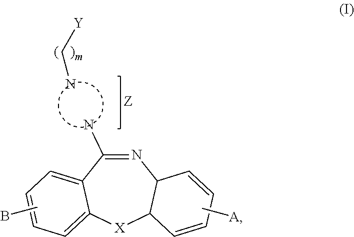
or a salt, complex, diastereomer, enantiomer and/or tautomer of thereof,
wherein
A is one or two substituents independently selected from —H, —F, —Cl, —Br, —I, —CF₃, —OCF₃, —CF₂H, and —OCF₂H;
B is one or two substituents independently selected from —F, —Cl, —Br, —I, —CF₃, —OCF₃, —CF₂H, and —OCF₂H;
X is selected from —S-and-O—;
m is 0 to 3;
Y is selected from
—H;
a nitrile group,
a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O; or
a linear —C₁₋₃alkyl, wherein a, preferably one, —(CH₂)— of the linear —C₁₋₃alkyl is optionally monosubstituted with —OH, and wherein the terminal —CH₃ of the linear-C₁₋₃alkyl is monosubstituted with —OR¹ or —SR¹, preferably —OR¹, wherein R¹ is H, —(CH₂)—COOH or —(CH₂)—(CH₂)—OH; or
—(CH₂)ᵢ—CX¹R², wherein
X¹ is ═NR³ or ═O, wherein R³ is —H or —OH.
i is 0 or 1, and
R² is —OR⁴ or —NR⁵R⁶, wherein R⁴ is —H or —C₁₋₂alkyl; wherein R⁵ and R⁶ are independently selected from —H or -methyl; 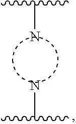
herein denoted as “Z”, is an aliphatic group comprising 1 or 2 heterocycles which comprise together two nitrogen atoms,
wherein the first of the two nitrogen atoms connects Z to 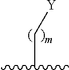
of formula I and the second of the two nitrogen atom connects Z to 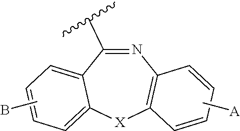
of formula I, preferably to 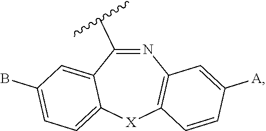
and wherein
the two nitrogen atoms are each a heteroatom in one of two connected 4-membered heterocycles (each heterocycle having no more than one heteroatom), wherein the two interconnected 4-membered heterocycles form a spirocylce, preferably wherein the 4-membered heterocycle with the first nitrogen atom is connected via a shared carbon atom to the 4-membered heterocycle with the second nitrogen atom, thus forming a spirocycle with two heteroatoms that are the two nitrogen atoms; or
the first of the two nitrogen atoms is connected to a 4-membered heterocycle with one heteroatom, wherein the second of the two nitrogen atoms is the heteroatom in the 4-membered heterocycle that the first of the two nitrogen atoms is connected to; wherein the first nitrogen atom is further substituted with methyl; or
the two nitrogen atoms are heteroatoms in a 6- to 7-membered heterocycle with two heteroatoms, wherein the heterocycle is optionally substituted, preferably monosubstituted, with methyl;
and wherein the compound is not any of

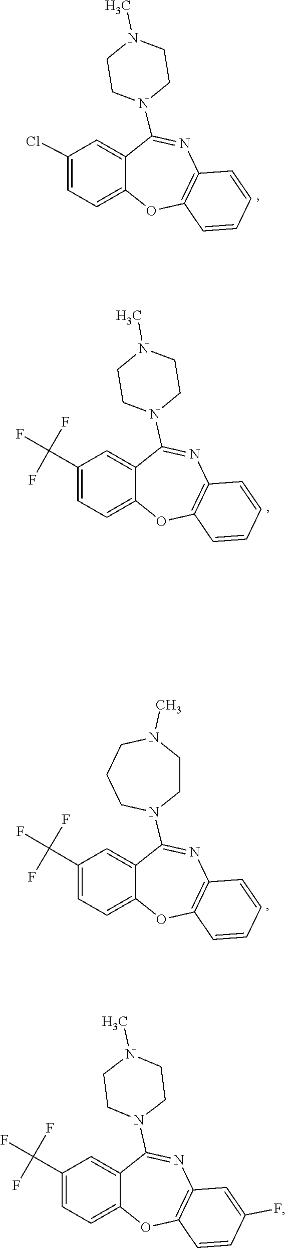 [SMILES: CN1CCN(C2=Nc3ccccc3Oc3ccc(Cl)cc32)CC1 ; CN1CCN(C2=Nc3ccccc3Oc3ccc(C(F)(F)F)cc32)CC1 ; CN1CCCN(C2=Nc3ccccc3Oc3ccc(C(F)(F)F)cc32)CC1 ; CN1CCN(C2=Nc3cc(F)ccc3Oc3ccc(C(F)(F)F)cc32)CC1]

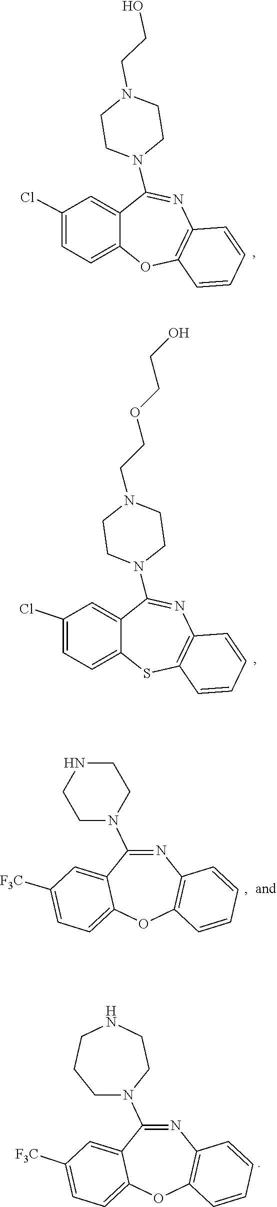 [SMILES: OCCN1CCN(C2=Nc3ccccc3Oc3ccc(Cl)cc32)CC1 ; OCCOCCN1CCN(C2=Nc3ccccc3Sc3ccc(Cl)cc32)CC1 ; FC(F)(F)c1ccc2c(c1)C(N1CCNCC1)=Nc1ccccc1O2 ; FC(F)(F)c1ccc2c(c1)C(N1CCCNCC1)=Nc1ccccc1O2]

2. The compound according to claim 1, wherein B is a substituent at the carbon positions 2 and 3, preferably B is the substituent —Cl at the carbon positions 2 and 3, when referring to the following numbering

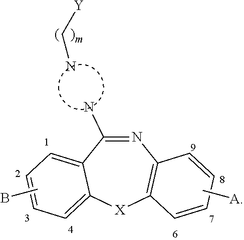

3. The compound according to claim 1, wherein A and B are each one substituent, preferably wherein the compound has the formula II

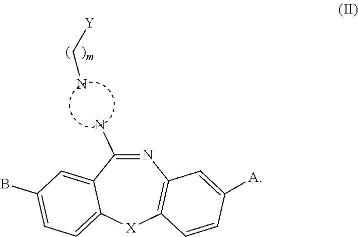

4. The compound of claims 1 to 3, wherein
(i) A is selected from —H or —F,
(ii) B is selected from —Cl or —CF₃,
(iii) m is 0 to 2, more preferably m is 0 or 1, most preferably m is 1,
(iv) Y is selected from the group consisting of 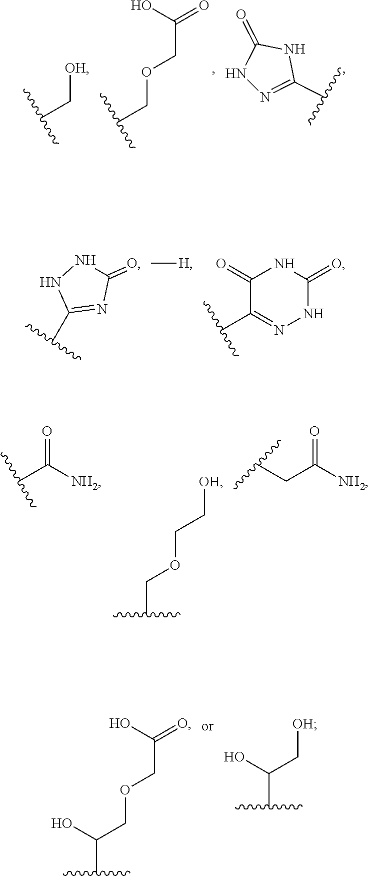
Z is selected from the group consisting of

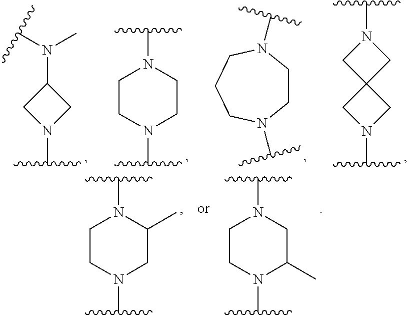

5. The compound according to any one of claims 1 to 4, wherein the compound is selected from the group consisting of compounds 2 to 14,

| Compound No. | Structure |
|---|---|
| 6 | 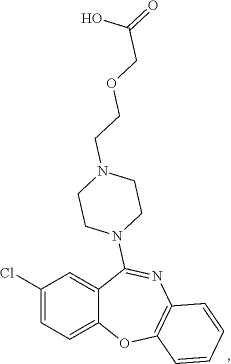 [SMILES: O=C(O)COCCN1CCN(C2=Nc3ccccc3Oc3ccc(Cl)cc32)CC1] |
| 8 | 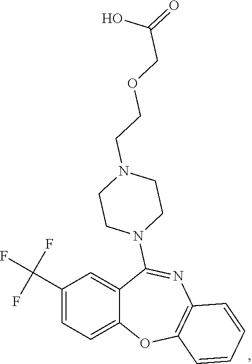 [SMILES: O=C(O)COCCN1CCN(C2=Nc3ccccc3Oc3ccc(C(F)(F)F)cc32)CC1] |
| 9 | 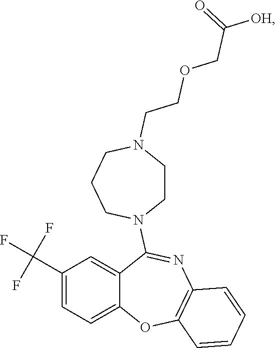 [SMILES: O=C(O)COCCN1CCCN(C2=Nc3ccccc3Oc3ccc(C(F)(F)F)cc32)CC1] |
| 10 | 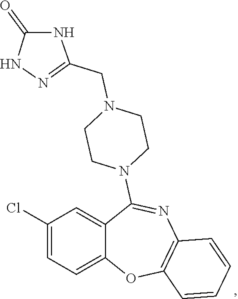 [SMILES: O=c1[nH]nc(CN2CCN(C3=Nc4ccccc4Oc4ccc(Cl)cc43)CC2)[nH]1] |
| 11 | 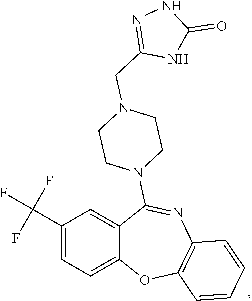 [SMILES: O=c1[nH]nc(CN2CCN(C3=Nc4ccccc4Oc4ccc(C(F)(F)F)cc43)CC2)[nH]1] |
| 12 | 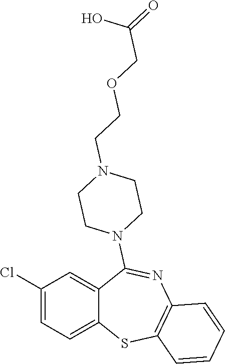 [SMILES: O=C(O)COCCN1CCN(C2=Nc3ccccc3Sc3ccc(Cl)cc32)CC1] |
| 15 | 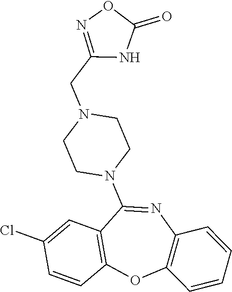 [SMILES: O=c1[nH]c(CN2CCN(C3=Nc4ccccc4Oc4ccc(Cl)cc43)CC2)no1] |
| 16 | 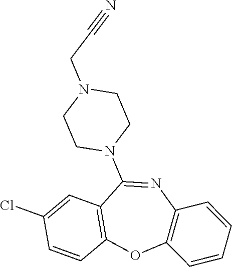 [SMILES: N#CCN1CCN(C2=Nc3ccccc3Oc3ccc(Cl)cc32)CC1] |
| 17 | 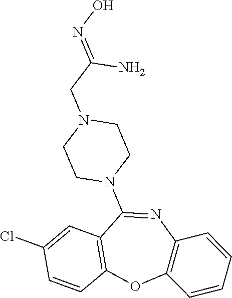 [SMILES: N/C(CN1CCN(C2=Nc3ccccc3Oc3ccc(Cl)cc32)CC1)=N\O] |
| 18 | 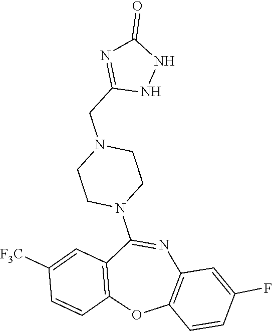 [SMILES: O=c1nc(CN2CCN(C3=Nc4cc(F)ccc4Oc4ccc(C(F)(F)F)cc43)CC2)[nH][nH]1] |
| 19 | 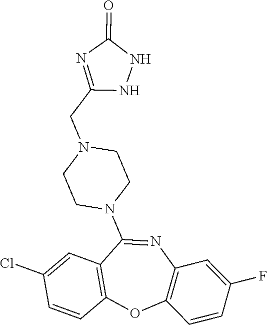 [SMILES: O=c1nc(CN2CCN(C3=Nc4cc(F)ccc4Oc4ccc(Cl)cc43)CC2)[nH][nH]1] |
| 20 | 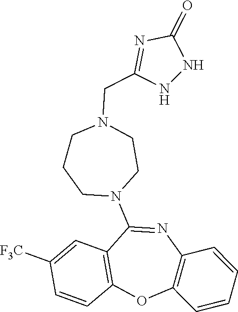 [SMILES: O=c1nc(CN2CCCN(C3=Nc4ccccc4Oc4ccc(C(F)(F)F)cc43)CC2)[nH][nH]1] |
| 21 | 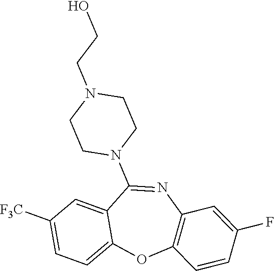 [SMILES: OCCN1CCN(C2=Nc3cc(F)ccc3Oc3ccc(C(F)(F)F)cc32)CC1] |

preferably wherein compound is selected from the group consisting of compounds 6, 8, 9, 10, 11, 12, 18, 19, 20, 21, more preferably the compound is selected from the group consisting of the compounds 6, 8, 9, and 10.

6. The compound of claims 1 to 5, wherein the compound has an activity as an activator of a potassium channel in a cell, preferably of a potassium channel Slack (K_{Na}1.1), preferably wherein the compound activates the potassium channel with an EC50 of less than 100 μM, preferable of between 0.5 and 80 μM.

7. The compound of any one of claims 1 to 6, wherein the compound
when administered to an animal does not penetrate the blood brain barrier (BBB), or wherein the compound penetrates the BBB less than a reference compound, such as Loxapine.
does not bind the human dopamine receptor, more preferably wherein the compound binds the human dopamine receptor less compared to a reference compound, such as Loxapine.

8. The compound of any one of claims 1 to 7, wherein the compound is isolated and/or wherein the compound has a purity of at least 75%, optionally at least 90%, optionally at least 95%.

9. A pharmaceutical composition comprising the compound of any one of claims 1 to 8, or a salt, solvate, or ester thereof, and a pharmaceutically acceptable carrier or excipient.

10. The pharmaceutical composition of claim 9, wherein the compound, or a salt, solvate, or ester thereof, is present in an amount of 0.1% w/w to 10% w/w, optionally in an amount of 1% w/w to 10% w/w.

11. A compound or composition for use in the treatment of a condition in a subject, the compound or composition comprising an effective amount of the compound of any one of claims 1 to 8, wherein the compound has the formula I: 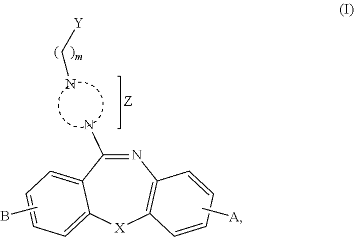
or a salt, complex, diastereomer, enantiomer and/or tautomer of thereof,
wherein
A is one or two substituents independently selected from —H, —F, —Cl, —Br, —I, —CF₃, —OCF₃, —CF₂H, and —OCF₂H;
B is one or two substituents independently selected from —F, —Cl, —Br, —I, —CF₃, —OCF₃, —CF₂H, and —OCF₂H;
X is selected from —S-and-O—;
m is 0 to 3;
Y is selected from
—H;
a nitrile group,
a 5- to 6-membered unsaturated heterocycle comprising 3 hetero atoms that are selected from O and N, preferably wherein 2 or 3 heteroatoms are nitrogen, wherein the heterocycle is mono or di-substituted with ═S or ═O, preferably with ═O; or
a linear —C₁₋₃alkyl, wherein a, preferably one, —(CH₂)— of the linear —C₁₋₃alkyl is optionally monosubstituted with —OH, and wherein the terminal —CH₃ of the linear-C₁₋₃alkyl is monosubstituted with —OR¹ or —SR¹, preferably —OR¹, wherein R¹ is H, —(CH₂)—COOH or —(CH₂)—(CH₂)—OH; or
—(CH₂)ᵢ—CX¹R², wherein
X¹ is ═NR³ or ═O, wherein R³ is —H or —OH.
i is 0 or 1, and
R² is —OR⁴ or —NR⁵R⁶, wherein R⁴ is —H or —C₁₋₂alkyl; wherein R⁵ and R⁶ are independently selected from —H or -methyl; 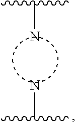
herein denoted as “Z”, is an aliphatic group comprising 1 or 2 heterocycles which comprise together two nitrogen atoms,
wherein the first of the two nitrogen atoms connects Z to 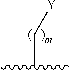
of formula I and the second of the two nitrogen atom connects Z to 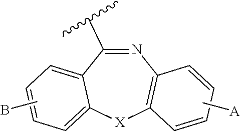
of formula I, preferably to 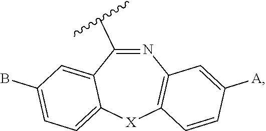
and wherein
the two nitrogen atoms are each a heteroatom in one of two connected 4-membered heterocycles (each heterocycle having no more than one heteroatom), wherein the two interconnected 4-membered heterocycles form a spirocylce, preferably wherein the 4-membered heterocycle with the first nitrogen atom is connected via a shared carbon atom to the 4-membered heterocycle with the second nitrogen atom, thus forming a spirocycle with two heteroatoms that are the two nitrogen atoms; or
the first of the two nitrogen atoms is connected to a 4-membered heterocycle with one heteroatom, wherein the second of the two nitrogen atoms is the heteroatom in the 4-membered heterocycle that the first of the two nitrogen atoms is connected to; wherein the first nitrogen atom is further substituted with methyl; or
the two nitrogen atoms are heteroatoms in a 6- to 7-membered heterocycle with two heteroatoms, wherein the heterocycle is optionally substituted, preferably monosubstituted, with methyl;
preferably the compound of any one of claims 1 to 8, or a salt, solvate, or ester thereof, preferably, preferably wherein the composition is the pharmaceutical composition of claim 9 or 10, wherein the condition is treatable by activating a potassium channel in a cell associated with the pathology of the condition, the treatment comprising administering the compound or composition to the subject in need thereof.

12. The compound or composition for use of claim 11, wherein the condition is a pruritic condition, such as an acute or chronic pruritus, optionally wherein the pruritic condition is associated with a second pathology, such as one selected from:
a dermatological disorder such as xerosis or xeroderma (dry skin), dermatitis or eczema (e.g., atopic dermatitis), psoriasis (e.g., plaque psoriasis), prurigo (e.g., prurigo nodularis), urticaria (e.g., chronic idiopathic urticaria), a connective tissue disorder (e.g., dermatomyositis), post-burn pruritus;
a kidney disorder (e.g., chronic kidney disease, chronic kidney failure or end-stage renal disease), dialysis (e.g., hemodialysis), uremic pruritus;
a hepato-biliary disorder (e.g., cholestasis, primary biliary cholangitis, primary sclerosing cholangitis, secondary sclerosing cholangitis, hepatitis, toxic liver disease, chronic liver disease or cirrhosis), cholestatic pruritus;
an endocrine disorder (e.g., hyperthyroidism or diabetes mellitus);
a metabolic disorder (e.g., iron deficiency or iron overload);
a benign or malignant neoplasm (e.g., a solid tumor, a carcinoma or a hematological neoplasm [e.g., Hodgkin's lymphoma, non-Hodgkin's lymphoma, myeloproliferative disease, or polycythemia vera]);
an infectious disease (e.g., a viral infection such as an infection with herpes simplex, herpes zoster, varicella, human immunodeficiency virus (HIV), hepatitis; a bacterial infection or a parasitosis);
a neurological disorder (e.g., a degenerative neurological disease, multiple sclerosis, a brain tumor, postherpetic neuralgia, small-fiber neuropathies, brachioradial pruritus or notalgia paresthetica), neuropathic itch, neurogenic itch;
a psychiatric disease (e.g., depression, obsessive compulsive disorder, delusional disorder, eating disorder, or anxiety);
drug-induced pruritus (e.g., by opioids, antibiotics, antimalarial agents, ACE inhibitors, angiotensin receptor antagonists, anti-arrhythmic agents, antidepressants, antidiabetic drugs, antihypertensive drugs, anticonvulsants, anti-inflammatory drugs, betablockers, bronchodilators, calcium antagonists, diuretics, hormones, immunosuppressive drugs, antilipids, neuroleptics, plasma expanders, tranquillizers, or uricostatics);
pruritus in the elderly;
pruritus in pregnancy; and/or
chronic idiopathic pruritus.

13. The compound or composition for use of claim 11 or 12, wherein the condition is pain or a pain related noxious sensation in the subject, preferably wherein the pain is selected from
neuropathic pain induced by traumatic nerve injury, cancer and cancer treatments (e.g., chemotherapy), neurological conditions (e.g., multiple sclerosis), neurodegenerative conditions (e.g., Parkinson's disease), trigeminal neuralgia, diabetic peripheral neuropathy, stroke, shingles, HIV, Hansen's disease (leprosy), Guillain-Barre syndrome, blood vessel disease, vascular malformations, autoimmune conditions
acute post-operative pain,
inflammatory pain, rheumatoid arthritis, osteoarthritis, or/and
nociplastic pain.

14. The compound or composition for use of claims 11 to 13, wherein administering comprises intravenous, intraperitoneal, subcutaneous, intramuscular, intrathecal, peridural, topical, oral, gastric and/or rectal administration.

15. A method of synthesizing a compound according to any one of claims 1 to 8, preferably, wherein the method comprises a synthesis step of synthesizing a lactam core, preferably wherein said lactam core comprises the structure: 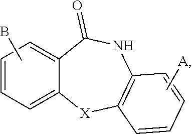
wherein A, X and B are defined as in any one of claims 1 to 8.


SEQUENCE LISTING (1 sequence)

SEQ ID NO: 1 - protein, 6 aa, synthetic construct
SLIGRL